(19) United States
(12) Patent Application Publication
(10) Pub. No.: US 2021/0348107 A1
(43) Pub. Date: Nov. 11, 2021

(54) DEVICE AND SYSTEMS COMPRISING ELECTRODE ARRAYS FOR ELECTROCONDUCTIVE CELLS

(71) Applicants: UNIVERSITY OF WASHINGTON, Seattle, WA (US); SAMSUNG LIFE PUBLIC WELFARE FOUNDATION, Seoul (KR)

(72) Inventors: Deok-Ho KIM, Seattle, WA (US); Byung-Ok CHOI, Seattle, WA (US); Alexander S.T. SMITH, Seattle, WA (US)

(73) Assignees: UNIVERSITY OF WASHINGTON, Seattle, WA (US); SAMSUNG LIFE PUBLIC WELFARE FOUNDATION, Seoul (KR)

(21) Appl. No.: 17/277,641

(22) PCT Filed: Sep. 19, 2019
(86) PCT No.: PCT/US2019/052004

§ 371 Date: Mar. 18, 2021

Related U.S. Application Data
(60) Provisional application No. 62/733,362, filed on Sep. 19, 2018.

(51) Int. Cl. C12M 1/42; C12M 1/34; G01N 33/50; G01N 33/483

(52) U.S. Cl. CPC C12M 35/02; G01N 33/4836; G01N 33/5058; C12M 41/46

(57) ABSTRACT

The technology described herein is directed to devices, systems, methods, and assays comprising electrode arrays for electroconductive cells. In particular, the technology generally relates to a microelectrode array (MEA) device comprising both field potential (FP) electrodes and impedance electrodes (IE) that are spatially separated for the functional analysis of the electrical connectivity between at least two cell populations, for example a plurality of neuronal cells and a plurality of contractile cells.

### CROSS-REFERENCE TO RELATED APPLICATIONS

This application claims benefit under 35 U.S.C. § 119(e) of U.S. Provisional Application No. 62/733,362 filed Sep. 19, 2018, the contents of which are incorporated herein by reference in their entirety.

### GOVERNMENT SUPPORT

This invention was made with government support under Grant No. R01 NS094388, awarded by the National Institutes of Health. The government has certain rights in the invention.

### TECHNICAL FIELD

The technology generally relates to the field of cell growth and tissue engineering. The technology described herein relates to devices, systems and assays comprising electrode arrays for at least two types of electroconductive cells.

### BACKGROUND

Peripheral neuropathies are a clinically and genetically heterogeneous set of severely debilitating neurological conditions. It has been suggested that the neuromuscular junction (NMJ) is an early target for pathological onset in a number of peripheral nerve diseases. For example, patients with amyotrophic lateral sclerosis (ALS) commonly possess defects in the maturation of their NMJs. These defects typically precede the onset of progressive muscular degeneration to the point that impairments in pre- and post-synaptic development in lower motor neuron connectivity to muscle is characteristic of early stages of the disease. It is therefore likely that defective NMJ maturation contributes to the subsequent onset of complete denervation and associated muscle weakness. Based on these data, it is clear that the NMJ is an important site of early, selective pathology in peripheral neuropathies, such as ALS. In order to prevent the degradation of synaptic structures, and improve patient mobility, it is vital to understand the upstream effects that lead to NMJ breakdown. Recent advances in human induced pluripotent stem cell (hiPSC) technology now makes phenotypic comparison of individual patient mutations a viable possibility. However, to evaluate the effect of different mutations, e.g., ALS mutations, on NMJ development requires a suitable high-throughput screening platform with which to evaluate synaptic function and breakdown.

In vitro models of NMJ physiology and function are currently available. They typically focus on dual patch analysis (see e.g., Umbach et al., PLoS One. 2012, 7: e36049) or assessment of muscle twitch responses to neuronal activation (see e.g., Smith et al., Technology 2013, 1:37-48; Guo et al., Biomaterials. 2011, 32:9602-11; Santhanam et al., Biomaterials. 2018, 166:64-78.). However, such systems are extremely low throughput and time consuming, preventing side by side comparison of multiple disease mutations as well as simultaneous analysis of multiple dose responses to novel therapeutics. A multiplexed, high-throughput NMJ functional screening platform is thus needed for the study of peripheral neuropathies.

Moreover, the culture of electroconductive cells, e.g. neurons and muscle cells, in a manner that permits measurement of their electroconductive behavior is important for understanding the biology of these cells, determining if candidate therapeutics perturb this behavior (possibly leading to dangerous side effects in patients), and identifying agents that can therapeutically modify electroconductive behaviors, e.g. for the treatment of arrhythmia. However, the existing technologies for culturing these cells do not discern the electrical properties from each cell type if more than one cell type is used, and suffer from suboptimal spatial accuracy, low-throughput, and alter the natural physiology and/or behavior of electroconductive cells due the characteristics of the culture environment.

In addition, culture systems and devices that permits the measurement of the electroconductive behavior are typically constructed for the evaluation of a single cell type at a time. While in some instances, the devices can be used for a spatially separated culture of two or more cell types, the different cell types are cultured together on the same surface, therefore preventing systematic analysis of the effect of one cell type on the other. A culture system that can serve as a functional screening platform for the analysis of the electroconductive behavior each cell type individually is needed for the study of peripheral neuropathies.

### SUMMARY

The technology described herein is directed to cell culture devices integrating electrode-based detection of neuronal firing and muscle contraction, particularly by compartmentalization of neuronal and muscle populations, and comprises bioinspired topographic substrates.

More particularly, the technology described herein relates to microelectrode array (MEA) devices, systems, methods, and assays comprising electrode arrays for electroconductive cells. In particular, the technology generally relates to MEA devices comprising both field potential (FP) electrodes and impedance electrodes (IE) that are spatially separated for the functional analysis of the electrical connectivity between at least two cell populations. Accordingly, the compositions and methods described herein relate to MEA devices for culturing and assessing at least two populations of electroconductive cells on a substrate, where the two cell populations are spatially separated. That is, each cell population attaches and adheres to a spatially distinct cell growth area on the substrate, and one or both cell types of each population can extend axons or other cellular projections into, and in some embodiments through, an intermediate area referred to herein as an “axon outgrowth area”, which is located between each of the cell growth areas such that cells of each cell population can connect electrically, e.g., make synaptic connections. That is, while the cell bodies of each cell population are spatially separated or distinct, cells from the different populations can connect or contact each other via axonal, dendritic or other projections from one cell type to the other cell type.

Therefore, by having the at least two cell populations spatially separated, yet permitting them to make synaptic connections to allow for electrical communication between the two cell types, it permits a flexible system in terms of both electrical stimulation and/or data collection capabilities. For example, one can electrically stimulate one cell type, and record the effect on the other cell type. Additionally, the MEA devices can be used in assays to see the effect of an agent on the electrical connectivity between the two cell types, when the agent is applied to one cell type, or both cell types. Moreover, the MEA devices disclosed herein can be used in assays and in vitro models for disease and disorders, e.g., neurodegenerative diseases, neuromuscular disorders and myopathies, to see the effect of a gene mutation on the electrical conductivity between the two cell types.

In some embodiments, such devices permit real-time analysis of NMJ development and function across a wide array of healthy and diseased cell lines. In some embodiments, primary human induced pluripotent stem cell-derived motor neurons and primary human muscle cells can be used to establish organized cultures overlying electrode beds for functional analysis. A barrier or wall can separate the cell populations into neuronal and muscle cell compartments. Microchannels in the surface of the device and/or voids in the barrier(s) separating the cells permit neuronal processes to grow from the neuronal cell compartment into the muscle compartment, facilitating synaptic contact. Drug compounds can be analyzed using this system and assessed with regard to their ability to ameliorate in vitro disease phenotypes. Such devices can be used to model human peripheral neuropathies and as a preclinical screening tool for assessing therapeutics.

In some embodiments, the technology described herein provides a platform for assessing neuromuscular junction functionality. The inventors discovered that to facilitate synapse development, rather than a co-culture of neurons cultured directly on top of muscle cells, the spatial separation of the two cell types permitted optimization for each cell type, as well as controlled stimulation of neurons and/or measurement of contractile responses of skeletal muscle to neuronal activation. As such, the inventors developed a cell surface platform integrating field potential electrodes on one side and impedance electrodes on the other, with an intervening cell surface area through which the neuronal axons extend to contact the muscle cells (see e.g., FIG. 1A). In some embodiments, neurons cultured on the field potential electrode side can be monitored for spontaneous activity and/or electrically stimulated to enhance output and strengthen synaptic connections. In some embodiments, a monolayer or multilayered skeletal muscle construct is plated on the surface which comprises impedance electrodes. Small scale electrical impulses can then be run between the impedance electrodes; these pulses can be too small to affect the overlying muscle, but large enough to permit quantification of impedance parameters. Other groups have demonstrated that contractile cells modulate the impedance associated with electrical pulses passing between electrodes that they cover. This modulation can be plotted over time to provide a surrogate measure of contractile ability, with the degree of impedance change correlating directly with contraction magnitude. A barrier (e.g., made from e.g., PDMS, among other materials) between the two compartments can permit isolation of each cell type and prevent migration of either cell type into the other population. Micro-scale channels permit neuritic extension from the neuron compartment to the muscle and thereby facilitate synapse development (see e.g., FIGS. 1B, 1C). Testing of electrode sensitivity demonstrates that the deposited electrodes are sufficiently sensitive to be used as described above (see e.g., FIGS. 1D, 1E) and bright-field imaging confirms the capacity for human iPSC-derived neurons to extend axons from one chamber, through the channels, and out into the second chamber (see e.g., FIG. 1F). The platform can be tested to validate its ability to support both cell types and assay functional connectivity between the muscle and neuronal components.

In some embodiments, tests with cholinergic synaptic agonists and antagonists in terms of their ability to alter synaptic communication between cell populations are used to demonstrate the suitability of this model for assaying NMJ function in vitro (see e.g., Example 1).

In some embodiments, this system incorporates motor neurons carrying different ALS mutations in one of at least three separate genes (e.g., TARDBP, SOD1, and C9ORF72). Spatially separated culture constructs incorporating these cells or cells bearing other ALS-related mutations can be used to model ALS disease pathophysiology in vitro and can be used to highlight phenotypic differences arising between motor neurons derived from different mutant hiPSC lines (see e.g., Example 2). In some embodiments, drug compounds can be analyzed using this system and assessed with regards to their ability to ameliorate in vitro disease phenotypes. (see e.g., Example 3).

In some embodiments, described herein is a device and system for the study of neuropathies such as Charcot-Marie-Tooth disease (CMT; see e.g., Example 4).

Without wishing to be bound by theory, it is proposed that differences in NMJ development and function between skeletal muscle and motor neurons derived from either normal or ALS (or other neuropathic disease-derived) hiPSC lines permits stratification of disease phenotypes that can be successfully assayed and identified using electrode-based, real-time monitoring. Devices and systems as described herein can be used to model human peripheral neuropathies and as a preclinical screening tool for assessing therapeutics.

In one aspect, described herein is a device for monitoring the electrical communication between two different electrically excitable cell types, comprising at least one module on a substrate, each module comprising: (a) a first cell growth area, a second cell growth area, and an axon outgrowth area flanked between the first cell growth area and the second cell growth area, and (b) a plurality of field potential electrodes and a plurality of impedance electrodes on the surface of the substrate, wherein the plurality of field potential electrodes are located on the surface of the first cell growth area and the plurality of impedance electrodes are located on the surface of second cell growth area, and wherein the plurality of field potential electrodes and the plurality of impedance electrodes are connected to an electronic interface.

In some embodiments of any of the aspects, the axon outgrowth area has a width of at least 100 μm between the first cell growth area and the second cell growth area.

In some embodiments of any of the aspects, the axon outgrowth area is configured for axons to extend from the first cell growth area to the second cell growth area.

In some embodiments of any of the aspects, the axon outgrowth area comprises a nanopattern on the substrate.

In some embodiments of any of the aspects, the nanopattern comprises a series of parallel grooves and ridges with a proximal and distal end, wherein the proximal end of the grooves and ridges interfaces with the first cell growth area, and the distal end of the grooves and ridges interfaces with the second cell growth area.

In some embodiments of any of the aspects, the groove depth is between 100 nm and 600 nm, and the ridge width is between 400 nm and 1000 nm.

In some embodiments of any of the aspects, the axon outgrowth area comprises a series of parallel microchannels with a proximal and distal end, wherein the proximal end of the microchannels interfaces with the first cell growth area, and the distal end of the microchannels interfaces with the second cell growth area.

In some embodiments of any of the aspects, the microchannel width is between 1 μm and 50 μm, the interchannel width is between 1 μm and 150 μm, and the microchannel depth is between 50 nm and 5000 nm.

In some embodiments of any of the aspects, the device further comprises at least one barrier located between the first cell growth area and the second cell growth area, wherein the barrier is configured to separate a plurality of cell bodies of cells located on the first cell growth area from cells located on the second growth area.

In some embodiments of any of the aspects, the barrier is located at the interface between the first cell growth area and the axon outgrowth area.

In some embodiments of any of the aspects, the barrier is located within the axon outgrowth area, and wherein the barrier is configured to separate a plurality of cell bodies of cells located on the first cell growth area from cells located on the second growth area.

In some embodiments of any of the aspects, the barrier is configured to allow axons from cells located on the surface of the first cell growth area to extend into the axon outgrowth area.

In some embodiments of any of the aspects, the barrier is a non-removable or removable physical barrier.

In some embodiments of any of the aspects, the barrier is the same width as the axon outgrowth area.

In some embodiments of any of the aspects, the plurality of field potential electrodes (FPE) is arranged in an array.

In some embodiments of any of the aspects, the plurality of impedance electrodes (IE) is arranged in an array.

In some embodiments of any of the aspects, the field potential electrodes (FPE) are configured to receive an electrical signal via the electrical interface from a power source and configured to deliver an electrical stimulating signal to the surface of the first cell growth area.

In some embodiments of any of the aspects, the field potential electrodes (FPE) are configured to monitor any one of: spontaneous, electrically-paced, or optically-paced activity of cells in contact with the field potential electrode.

In some embodiments of any of the aspects, the field potential electrodes (FPE) are configured for one or both of: (a) monitor any one of: spontaneous, electrically-paced, or optically-paced activity of cells in contact with the field potential electrode; and (b) electrically stimulate cells present on the first cell growth area.

In some embodiments of any of the aspects, the field potential electrodes can electrically stimulate synaptic connections of cells present on the first cell growth area with the cells present on the second growth area.

In some embodiments of any of the aspects, the impedance electrodes (IE) are communicatively coupled via the electrical interface to at least one analyzing module in the form of an impedance analyzer, thereby permitting impedance monitoring from excitable cells attached to the surface of the second growth area.

In some embodiments of any of the aspects, the device further comprises a third cell surface area, wherein an edge of the third cell surface area interfaces with a proximal edge of the first cell growth area and the axon outgrowth area, or a distal edge of the second cell growth area and the axon outgrowth area.

In some embodiments of any of the aspects, the device further comprises a plurality of neuronal cells on the first cell growth area and a plurality of contractile cells or muscle cells on the second cell growth area.

In some embodiments of any of the aspects, the plurality of muscle cells on the second cell growth area is a 2D monolayer or an engineered 3D skeletal muscle construct.

In some embodiments of any of the aspects, the plurality of neuronal cells is derived from iPSCs obtained from a healthy subject or a subject with a neurodegenerative disease or disorder or a myopathy.

In some embodiments of any of the aspects, the plurality of muscle cells is derived from iPSCs obtained from a healthy subject or a subject with a neurodegenerative disease or disorder or a myopathy.

In some embodiments of any of the aspects, the plurality of neuronal cells comprise motor neurons, and the plurality of muscle cells comprise skeletal muscle cells.

In some embodiments of any of the aspects, the plurality of neuronal cells comprise sympathetic neurons, and the plurality of muscle cells comprise smooth muscle cells.

In some embodiments of any of the aspects, the plurality of neuronal cells comprise sympathetic neurons, and the plurality of muscle cells comprise cardiac muscle cells or cardiomyocytes.

In some embodiments of any of the aspects, the device further comprises an additional cell type on any one or more of: the first cell growth area, the second cell growth area or the axon outgrowth area.

In some embodiments of any of the aspects, the additional cell type is selected from any of: Schwann cells, microglia, astrocytes or satellite cells.

In some embodiments of any of the aspects, the additional cell type is Schwann cells, wherein the Schwann cells are located on the first cell growth area or axon outgrowth area, or both.

In some embodiments of any of the aspects, the neurodegenerative disease or a myopathy is selected from any of: CMT, ALS, SMA, myasthenia gravis, DMD, and a neuromuscular disease or wasting disorder.

In some embodiments of any of the aspects, the neuronal cells on the first cell growth area extend axons through the axon outgrowth area and into the second cell growth area comprising a plurality of muscle cells.

In some embodiments of any of the aspects, the neuronal cells that extend axons through the axon outgrowth area make synaptic connections with muscle cells present on the second growth area.

In some embodiments of any of the aspects, the first cell growth area comprises a nanopatterned surface, or the second cell growth comprises a nanopatterned surface, or both the first and the second cell growth surfaces comprise a nanopatterned surface.

In some embodiments of any of the aspects, the nanopattern on the first cell growth area is the same as the nanopattern on the second cell growth area.

In some embodiments of any of the aspects, the nanopattern on the first cell growth area is different to the nanopattern on the second cell growth area.

In some embodiments of any of the aspects, the nanopatterned surface provides anisotropic cues that promote improved levels of maturation of neuronal cell types, motor neurons and myocytes.

In some embodiments of any of the aspects, the device comprises an array of modules on the substrate.

In one aspect described herein is a system for measuring the electrical conductance from one cell type to a second cell type comprising: (a) a device as described herein, (b) an electronic interface for stimulation of field potential electrodes on the device, and (c) an electronic interface that permits recording of electrical activity from the impedance electrodes.

In one aspect described herein is a method for measuring the electrical conductance from one cell type to a second cell type comprising: (a) providing a device as described herein, wherein the device comprises a first cell type on the first cell growth area, and a second cell type on the second cell growth area, and wherein the first cell type extends axons across the axon outgrowth area to the second cell type in the second cell growth area; (b) providing electrical stimulation to the first cell type via the field potential electrodes, and (c) recording electrical activity of the second cell type via the impedance electrodes.

In some embodiments of any of the aspects, the first cell type is a plurality of neuronal cells.

In some embodiments of any of the aspects, the second cell type is a plurality of contractile or muscle cells.

In some embodiments of any of the aspects, the plurality of muscle cells is a monolayer or an engineered skeletal muscle construct.

In some embodiments of any of the aspects, the plurality of neuronal cells is derived from iPSCs obtained from a healthy subject or a subject with a neurodegenerative disease or disorder or a myopathy.

In some embodiments of any of the aspects, the plurality of muscle cells is derived from iPSCs obtained from a healthy subject or a subject with a neurodegenerative disease or disorder or a myopathy.

In some embodiments of any of the aspects, the plurality of neuronal cells comprise motor neurons, and the plurality of muscle cells comprise skeletal muscle cells.

In some embodiments of any of the aspects, the plurality of neuronal cells comprise sympathetic neurons, and the plurality of muscle cells comprise smooth muscle cells.

In some embodiments of any of the aspects, the plurality of neuronal cells comprise sympathetic neurons, and the plurality of muscle cells comprise cardiac muscle cells or cardiomyocytes.

In some embodiments of any of the aspects, the plurality of neuronal cells, or plurality of muscle cells, or both, are genetically modified cells to introduce one or more mutations for a neurodegenerative disease or myopathy.

In some embodiments of any of the aspects, the plurality of neuronal cells, or plurality of muscle cells, or both, are isogenic controls of a genetically modified cell that has one or more mutations introduced for a neurodegenerative disease or myopathy.

In some embodiments of any of the aspects, a method as described herein is used to assess electrical conductance across at least one neuromuscular junctions (NMJ) between an axon extended from the first cell type and the cell bodies of the second cell type.

In one aspect described herein is an assay for assessing an agent for modulation of electrical signaling from one cell type to another cell type, comprising: (a) providing a device as described herein, wherein the device comprises a first cell type located on surface of the first cell growth area, and a second cell type located on the second cell growth area, and wherein the first cell type extends axons across the axon outgrowth area from the first cell area to the second cell type in the second cell growth area; (b) contacting the first cell type, second cell type, or both, with an agent; (c) providing electrical stimulation to the first cell type via the field potential electrodes; (d) recording electrical activity of the second cell type via the impedance electrodes; and (e) detecting a change in electrical activity of the second cell type recorded via the impedance electrodes in the presence of the agent as compared to the absence of the agent.

In some embodiments of any of the aspects, the first cell type is a plurality of neuronal cells.

In some embodiments of any of the aspects, the second cell type is a plurality of contractile or muscle cells.

In some embodiments of any of the aspects, the plurality of muscle cells is a monolayer or an engineered skeletal muscle construct.

In some embodiments of any of the aspects, the plurality of neuronal cells is derived from iPSCs obtained from a healthy subject or a subject with a neurodegenerative disease or disorder or a myopathy.

In some embodiments of any of the aspects, the plurality of muscle cells is derived from iPSCs obtained from a healthy subject or a subject with a neurodegenerative disease or disorder or a myopathy.

In some embodiments of any of the aspects, the plurality of neuronal cells comprise motor neurons, and the plurality of muscle cells comprise skeletal muscle cells.

In some embodiments of any of the aspects, the plurality of neuronal cells comprise sympathetic neurons, and the plurality of muscle cells comprise smooth muscle cells.

In some embodiments of any of the aspects, the plurality of neuronal cells comprise sympathetic neurons, and the plurality of muscle cells comprise cardiac muscle cells or cardiomyocytes.

In some embodiments of any of the aspects, the plurality of neuronal cells, or plurality of muscle cells, or both, are genetically modified cells to introduce one or more mutations for a neurodegenerative disease or myopathy.

In some embodiments of any of the aspects, the plurality of neuronal cells, or plurality of muscle cells, or both, are isogenic controls of a genetically modified cell that has one or more mutations introduced for a neurodegenerative disease or myopathy.

In some embodiments of any of the aspects, an assay as described herein can assess neuromuscular junctions (NMJ) between the axons from the first cell type and the cell bodies of the second cell type.

### BRIEF DESCRIPTION OF THE DRAWINGS

FIG. 1A-1F is a series of images and graphs showing a compartmentalized chip for assessing neuromuscular junction functionality. FIG. 1A is a macroscopic top-view image of a fabricated chip supporting field potential and impedance electrodes. FIGS. 1B and 1C are scanning electron micrographs of PDMS channels used to create separate culture channels between field potential and impedance electrodes, for example for maintenance and analysis of motor neurons and skeletal muscle, respectively. Such side views are a magnification of the dashed rectangle indicated in FIG. 1A. FIG. 1D is a line graph showing an assessment of impedance electrode sensitivity in response to increasing frequency of test pulses on the device. FIG. 1E is a line graph showing measurement of voltage recorded from field potential electrodes in the device following input of 5 V test pulses. FIG. 1F is a bright-field image of neurites from induced pluripotent stem cell (iPSC) derived motor neurons growing through PDMS channels on the device. Cells were seeded in the left-hand chamber and neurite extension can clearly be observed emerging from the channels in the right-hand channel (white arrows).

FIG. 2A-2E is a series of schematics, images, and graphs showing electrode layout, deposition, and functional testing of one embodiment of the device. FIG. 2A is a schematic showing electrode layout for a single well of a combinatorial field potential and impedance electrode device. FIG. 2B is a magnified view of the dashed rectangle indicated in FIG. 2A, showing detail of the electrode design. The pitch between field potential electrodes is 200 μm. FIG. 2C is a top-view image of a fabricated electrode plate. FIG. 2D is a bright-field image of human motor neurons grown on in-house fabricated electrodes. FIG. 2E is a line graph showing a whole cell patch clamp recording from neuron grown on in-house fabricated electrodes. Once a patch was achieved, stepwise increases in voltage were applied through the underlying electrode. Action potential firing was observed when electrode stimulation reached 2.6 V.

FIG. 3 is a schematic of an exemplary device and an exemplary array.

FIG. 4 is a schematic showing Nafion nanopattern generation on electrode arrays. Pristine wells are first treated with poly(3,4-ethylenedioxythiophene) (PEDOT) to improve the sensitivity of the base electrode. A drop of Nafion resin is then applied to the substrate, and a PDMS mold is pressed into it. After overnight curing, the PDMS mold is removed to reveal Nafion topographic substrates underneath. When used in culture, Nafion nanopatterns promote uniaxial cellular alignment.

FIG. 5A-5D is a series of schematics and images showing compartmentalized chamber design. FIG. 5A is a schematic showing a computer-aided design (CAD) for compartmentalized chambers. 1 indicates the barrier void opening (e.g., 5 μm). 2 indicates the inter-void spacing of the barrier (e.g., 20 μm). 3 indicates barrier thickness (e.g., 1 mm). 4 indicates chamber wall height (e.g., 250 μm). 5 indicates chamber wall thickness (e.g., 2-3 mm). 6 indicates outer wall length (e.g., 20 mm). FIG. 5B-5C are SEM top-view images of increasing magnification illustrating the neurite channels for separating the two soma compartments. FIG. 5D is a bright field image of a compartmentalized device on glass slide with SH-SY5Y cells grown in one compartment. Analysis clearly indicates neuritic extension within the PDMS channels (white arrows).

FIG. 6A-6F is a series of images and graphs showing neuron and skeletal muscle function on flat and patterned multielectrode arrays (MEAs). FIG. 6A is a low magnification image of a multiwell MEA plate with nanotopography applied to each well. FIG. 6B is a magnified image of FIG. 6A showing Nafion nanotopography applied to a single well of a 48-well MEA plate. The presence of the nanoscale features causes light diffraction on the surface, giving the patterns a distinct shade in this image (white arrow). FIG. 6C is an SEM image of Nafion nanotopography, illustrating the high level of uniformity achieved when using the described fabrication method. FIG. 6D is a bar graph showing the spontaneous firing rate weighted to active electrodes in human iPSC (hiPSC) derived neuronal cultures maintained on flat and patterned MEAs. FIG. 6E is a bar graph showing the spontaneous firing rate in primary human skeletal muscle cultures maintained on flat and patterned MEAs. FIG. 6F is a bar graph showing the spontaneous firing rate in primary human skeletal muscle with and without motor neuron co-cultures.

FIG. 7A-7B is a series of graphs showing the use of impedance electrodes to indicate force production in hiPSC muscle cultures. FIG. 7A shows a representative trace from 3 separate wells of a device showing impedance-based measurement of contraction (top three graphs) and field potential measurements (bottom three graphs) in monolayers of hiPSC-derived cardiomyocytes. The traces are ordered so that each contraction matches the correspondingly ordered field potentials. FIG. 7B shows representative traces from the same 3 wells as FIG. 7A following treatment with blebbistatin to inhibit contraction.

FIG. 8A-8H is a series of images and graphs showing motor neuron production from UC2 iPSCs. FIG. 8A-8C shows representative bright field images of cells at different stages of differentiation. FIG. 8D shows day 40 neurons stained for neurofilament (NF). FIG. 8E shows day 40 neurons stained for Islet-1 expression. FIG. 8F shows flow cytometry results for unstained cells (left-most peak), secondary only labeled cells (middle peak), and cells labeled with FIT-C conjugated Islet-1 (right-most peak). Islet-1 positive cells made up 34.3% of the population. FIG. 8G shows a representative voltage clamp recording from whole cell patch of day 40 neuron showing characteristic inward and outward currents. FIG. 8F shows a representative current clamp recording from whole cell patch of day 40 neuron showing characteristic repetitive action potential firing behavior.

FIG. 9A-9C is a series of graphs showing electrophysiological assessment of hiPSC-derived neuronal function. FIG. 9A shows resting membrane potentials measured from normal (n=25) and TDP-43 mutant (M337V: n=20, and Q331K: n=20) neurons by whole cell patch clamp. *p=0.002. FIG. 9B shows representative action potential firing properties recorded from hiPSC-derived neurons, indicating increasing functional maturation. FIG. 9C is a bar graph showing the percentage of cells from normal (n=43), M337V mutant (n=20), and Q331K mutant (n=25) neuron populations exhibiting different action potential firing behavior during whole cell patch clamp analysis. A contingency table (data not shown) comparing cell type against firing behavior found that the two factors are significantly related (p=0.002), suggesting that frequency of firing type occurrence within each population is dependent on the cell type examined.

FIG. 10A-10C is a series of bar graphs showing increased ROS and oxidative stress in motor neurons expressing TDP-43 Q331K. Normal and TDP-43 Q331K mutant motor neurons were labeled with fluorescent dye indicators of (FIG. 10A) oxidative stress, (FIG. 10B) superoxide, and (FIG. 10C) nitric oxide (AbCam). Images from confocal fluorescence microscopy were analyzed to determine pixel intensity as a measure of relative expression levels. In all presented data, *p<0.01.

FIG. 11 is a series of images and bar graphs showing phenotypic analysis in both CMT-iPSC and hESC-derived motor neurons. CMT2 motor neurons express motor neuron specific transcription factors HB9 and Islet 1/2 (ISL1/2), as well as neuronal cytoskeletal markers such as the neuron specific β-III-tubulin (TuJ1), un-phosphorylated neurofilament H (SMI32), and microtubule associated protein 2 (MAP-2). CMT2 motor neurons also expressed choline acetyltransferase (ChAT) and the synaptic vesicular marker, synapsin. Scale bars: 100 μm. All iPSC sources were equally capable of differentiation as measured by SMI32 (top bar graph) and MAP2 (bottom bar graph) positivity.

FIG. 12A-12G is a series of images and graphs showing robust production and functional analysis of CMT2D patient-derived motor neurons. FIG. 12A shows an immunostained image of motor neuron population produced from iPSCs after 27 days in vitro. FIG. 12B shows representative traces of action potential (AP) firing behavior. FIG. 12 C is a bar graph showing quantification of AP firing behavior in control (WTC11) iPSC-derived motor neurons and neurons differentiated from iPSCs derived from a GARS mutant patient after 25 days in vitro. FIG. 12D shows an immunostained image of motor neuron population after 24 days in vitro and 1 day after MACS purification. FIG. 12E is a bar graph showing quantification of Islet-1 positive cells 1 day after MACS purification. *p<0.05. FIG. 12F shows an immunostained image of MACS purified motor neuron population produced from iPSCs after 50 days in vitro. FIG. 12G shows a high-magnification immunostained image of MACS purified motor neuron population produced from iPSCs after 50 days in vitro.

FIG. 13A-13D is a series of images and graphs showing characterization of an engineered isogenic control line for GARS mutant iPSCs derived from a patient with CMT2D. FIG. 13A shows sequencing results from GARS mutant line (left) and corrected isogenic line (right) illustrating successful correction of point mutation in the engineered control. FIG. 13B shows immunostained images of GARS isogenic control colonies illustrating expression of typical markers of pluripotency in this line. FIG. 13C shows FACS result from GARS isogenic control iPSCs illustrating high (97%) purity of cells expressing the pluripotent marker SSEA4. FIG. 13D shows GARS isogenic control line karyotyping result illustrating the maintenance of a normal karyotype after genetic editing of GARS point mutation in this line.

FIG. 14A-14C shows Table 4, which includes non-limiting examples of nanogroove dimensions. Table 4 includes exemplary combinations of nanogroove width (nm), ridge width (nm), and depth (nm).

FIG. 15A-15C shows Table 5, which includes non-limiting examples of microchannel dimensions. Table 5 includes exemplary combinations of microchannel width (micrometers, um), interchannel spacing/width (micrometers, um), and microchannel depth (nm). Please note that the length of the microchannel is determined by width of axon extension area, or distance between the first cell growth area and the second cell growth area.

### DETAILED DESCRIPTION

Embodiments of the technology described herein relate to devices, systems, methods, and assays comprising electrode arrays for electroconductive cells. In particular, the technology generally relates to an electrode array or a microelectrode array (MEA) device comprising both field potential (FP) electrodes and impedance electrodes (IE) that are spatially separated for the functional analysis of the electrical connectivity between at least two cell populations. For simplicity, the electrode array or microelectrode array devices will be referred to herein as MEA devices.

Accordingly, the compositions and methods described herein relate to an MEA device for culturing and assessing at least two populations of electroconductive cells on a substrate, where the two cell populations are spatially separated. That is, each cell population attaches and adheres to a spatially distinct cell growth area on the substrate, and one or both cell types of each population can extend axons or other cellular projections into, and in some embodiments through, an intermediate area referred to herein as an “axon outgrowth area”, which is located between each of the cell growth areas such that cells of each cell population can connect electrically, e.g., make synaptic connections. That is, while the cell bodies of each cell population are spatially separated or distinct, cells from the different populations can connect or contact each other via axonal, dendritic or other projections from one cell type to the other cell type.

Therefore, by having the at least two cell populations spatially separated, yet permitting them to make synaptic connections to allow for electrical communication between the two cell types, it permits a flexible system in terms of both electrical stimulation and/or data collection capabilities. For example, one can electrically stimulate one cell type, and record the effect on the other cell type. Additionally, the MEA devices can be used in assays to evaluate the effect of an agent on the electrical connectivity between the two cell types, when the agent is applied to one cell type, or both cell types. Moreover, the MEA device disclosed herein can be used in assays and in vitro models for diseases and disorders, e.g., neurodegenerative diseases, neuromuscular disorders and myopathies, to evaluate the effect of a gene mutation on the electrical conductivity between the two cell types.

Accordingly, one aspect of the technology disclosed herein relates to the use of the MEA devices in methods and assays for the differentiation, maturation and functional analysis of electroconductive cells, including muscle cells (including, but not limited to, cardiomyocytes, skeletal muscle myocytes and smooth muscle myocytes) and neuronal cells. The electrodes present on or adjacent to the cell culturing surface of each cell type can be used to stimulate and record from cells cultured on the surface. Additionally, the MEA devices provided herein allow high-throughput stimulation and data collection with a resolution not previously possible.

In some embodiments, these MEA devices can comprise nanopatterned surface to support the growth or maturity of electroconductive cells or promote the growth or maturation of cells that exhibit behaviors which are more physiologically relevant than prior methods allowed (e.g., increased force generation and/or increased field potentials).

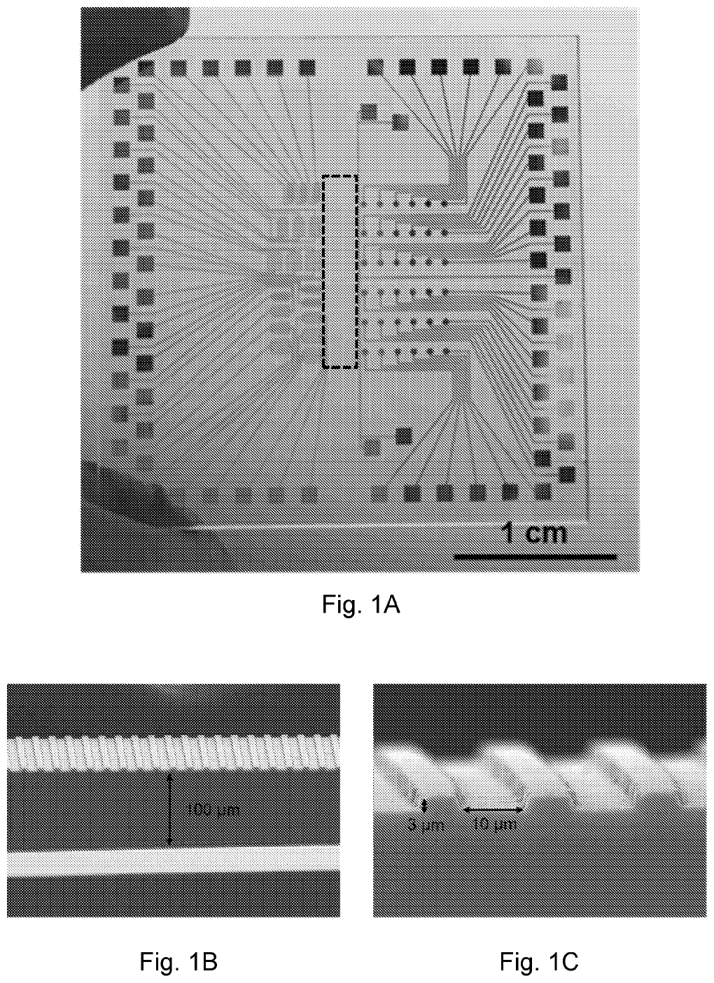

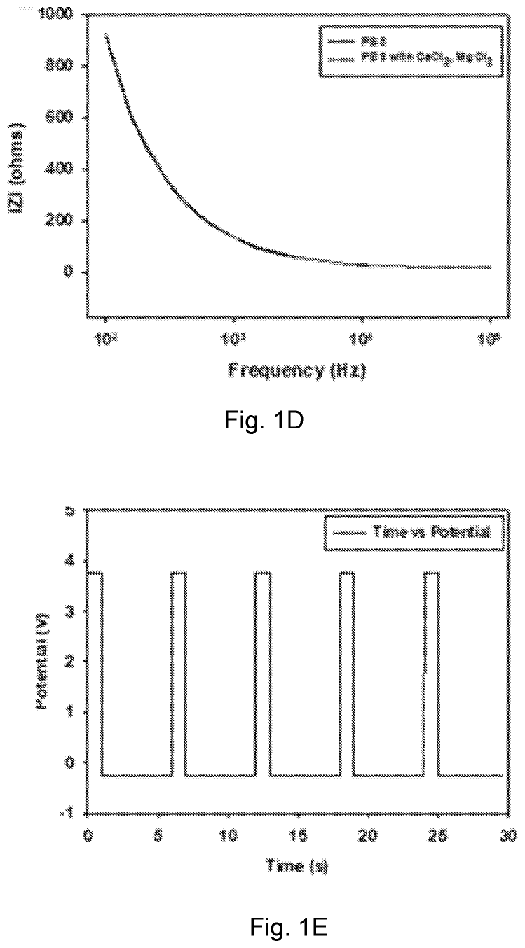

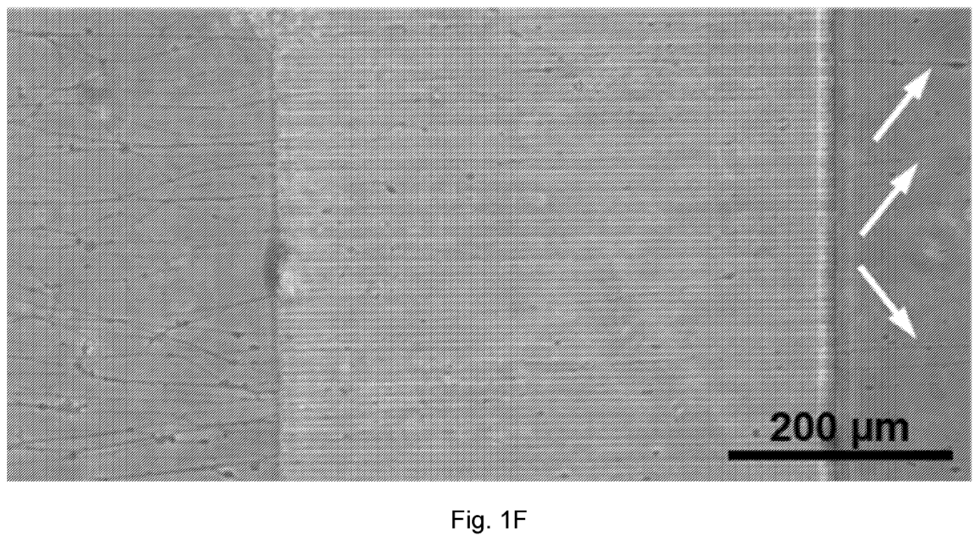

In some embodiments, the technology described herein provides a platform for assessing neuromuscular junction (NMJ) functionality. The inventors discovered that to facilitate synapse development, rather than a co-culture of neurons cultured directly on top of muscle cells, the spatial separation of the two cell types permitted optimization for each cell type, as well as controlled stimulation of neurons and/or measurement of contractile responses of skeletal muscle to neuronal activation. As such, the inventors developed a cell surface platform integrating field potential electrodes on one side and impedance electrodes on the other, with an intervening cell surface area through which the neuronal axons extend to contact the muscle cells (see e.g., FIG. 1A). In some embodiments, neurons cultured on the field potential electrode side can be monitored for spontaneous activity and/or be electrically stimulated to enhance output and strengthen synaptic connections. In some embodiments, a monolayer or multilayered skeletal muscle construct is plated on the surface which comprises impedance electrodes. Small scale electrical impulses can then be run between the impedance electrodes; these pulses can be too small to affect the overlying muscle, but large enough to permit quantification of impedance parameters. Other groups have demonstrated that contractile cells modulate the impedance associated with electrical pulses passing between electrodes that they cover. This modulation can be plotted over time to provide a surrogate measure of contractile ability, with the degree of impedance change correlating directly with contraction magnitude. A barrier (e.g., made of PDMS or other polymers or substances) between the compartments can permit isolation of each cell type and prevent migration of either cell type into the other population. Micro-scale channels permit neuritic extension from the neuron compartment to the muscle and thereby facilitate synapse development (see e.g., FIGS. 1B, 1C). Testing of electrode sensitivity demonstrates that the deposited electrodes are sufficiently sensitive to be used as described above (see e.g., FIGS. 1D, 1E) and bright-field imaging confirms the capacity for human iPSC-derived neurons to extend axons from one chamber, through the channels, and out into the second chamber (see e.g., FIG. 1F). The platform can be tested to validate its ability to support both cell types and assay functional connectivity between the muscle and neuronal components.

In some embodiments, the devices, systems, methods, and assays described herein can be used to study a range of peripheral neurodegenerative diseases and/or myopathies, including for example, but not limited to motor neuron diseases such as amyotrophic lateral sclerosis (ALS), which is a devastating peripheral neuropathy, characterized by motor neuron death, leading to muscular atrophy and eventual paralysis. Although the genetics that underpin ALS onset and progression are complex, an early pathological event is the retraction of distal motor axons and the breakdown of neuromuscular junctions (NMJs) in the extremities. Over the past decade, remarkable advances in the understanding of the cellular and molecular mechanisms underpinning neuropathic phenotype development have been made through in-depth studies of NMJs. However, knowledge gained through study of model systems has limitations in terms of accurately recapitulating mature human synapse pathophysiology. Specifically, the understanding of pathological ALS mechanisms has been advanced through investigation of animal models, but the availability of human biopsies or post-mortem samples is limited, which in turn restricts the study of patient specific mutations and their direct impact on disease etiology. An inability to accurately model human tissues carrying different patient mutations limits the capacity to identify common early defects in such patients, thereby reducing the ability to identify suitable upstream targets for therapeutic intervention.

Accordingly, described herein is a high-throughput model of innervated human skeletal muscle that in some embodiments can be used to evaluate early phenotypic defects in neurodegenerative diseases (e.g., ALS or Charcot-Marie-Tooth disease (CMT)), and can serve as a test bed with which to evaluate the efficacy of therapeutic strategies. As described in some embodiments, a system utilizing human induced pluripotent stem cell (hiPSC)-derived motor neurons from ALS or CMT patients not only improves the understanding of relevant disease mechanisms, but it also represents a valuable new platform for preclinical screening of drugs with the potential to ameliorate a wide range of peripheral neuropathic symptoms.

In some embodiments, described herein is a high-throughput, compartmentalized platform for investigating neuromuscular connectivity and synaptic function in human hiPSC-derived nerve or muscle cultures, which are compartmentally separated, yet synaptically connected. In some embodiments, the high-throughput system can be used to evaluate the in vitro phenotype and drug responses of a wide range of compartmentalized, yet synaptically connected hiPSC-derived neuronal-muscle cultures from a plurality of healthy and diseased patient cells.

As described herein, a compartmentalized design is used to separate motor neuron and skeletal muscle cell soma, with microchannels to permit axonal growth from the neuronal compartment to the musculature. Field potential electrodes embedded into the neuronal chamber permit real-time recording of burst fire behavior in overlying cells as well as stimulation of cultured cells in a controlled firing pattern. Impedance sensing electrodes in the muscle chamber are used to detect changes in electrical resistance in cell monolayers caused by neuronally-controlled contractile activity. In some embodiments, a series of neuromuscular stimulants and inhibitors can be used to detect modulation of NMJ communication using the systems as described herein (see e.g., Example 1).

In some embodiments, the devices and systems as described herein are used to investigate the functional effect of incorporating neurons carrying a neurodegenerative disease causing- or associated-mutation (e.g., ALS or CMT-relevant mutation) into a compartmentalized assay system. Without wishing to be bound by theory, it is proposed that ALS mutant neurons exhibit mutation specific functional phenotypes that are evident within the functional screening platform as described herein. In some embodiments, CRISPR/Cas9 gene editing can be used to generate ALS-relevant mutations in normal hiPSC populations to produce diseased cells and isogenic controls. As a non-limiting example, cells can be engineered comprising at least two pathogenic point mutations in TARDBP (e.g., mild (M337V) and severe (Q331K) mutations), at least one mutation in SOD1 (G93A), or at least one mutation in C9orf72. Motor neurons and skeletal muscle cells can be generated from these lines and their isogenic control counterparts for use in the high throughput functional screening system as described herein. Changes in connectivity, measured by the number of synchronous neuronal and muscular activations, can be compared across lines in order to establish whether ALS mutations lead to significant changes in NMJ formation within the in vitro assay as described herein (see e.g., Example 2).

In some embodiments, the devices and systems can be used to assess the capacity for therapeutic compounds to restore normal synaptic function in nerve-muscle spatially separated cultures. Without wishing to be bound by theory, it is proposed that ALS targeted compounds permit a return to normal in vitro phenotypes in mutant lines maintained within an in vitro screening system. In addition to CRISPR gene-edited lines, ALS patient-derived iPSC lines carrying any of a wide array of mutations in numerous causative genes can be used. Using such cells in the screening systems, assays, and methods described herein, compounds such as but not limited to those in ALS clinical trials can be investigated for their ability to reverse defective synaptic development and functional performance in mutant cultures.

The devices and methods described herein incorporate the fields of stem cell biology, bioengineering, neuromuscular biology, and clinical neurology. The device as described herein is a first-of-its-kind, high-throughput, functional screening platform for evaluating e.g. ALS patient mutations in terms of their effect on neuromuscular synaptic development. Furthermore, these devices, systems, methods, and assays described herein provide a framework for the preclinical study of new compounds and a more predictive model for evaluating the capacity of these drugs to ameliorate symptomatic progression in patients suffering from this and other severely debilitating and/or even life-threatening conditions.

### I. MEA Device

The compositions and methods described herein relate to an electrode array (MEA) device for the culture of at least two populations of electroconductive cells on a substrate, where each of the cell populations of the electroconductive cells are spatially separated. That is, each cell population attaches and adheres to spatially distinct cell growth areas on the substrate, and one or both cell types of each population can extend axons or other cell projections into an intermediate area, referred to as an “axon outgrowth area” located between each of the cell growth areas such that they can connect electrically, or make synaptic connections. That is, while each cell population is spatially separated or distinct, they can connect or touch via axonal, dendritic or other projection from one cell type to the other. Therefore, by having the at least two cell populations spatially separated, yet permitting them to make synaptic connections to allow for electrical communication between the two cell types, it permits a flexible system in terms of both electrical stimulation and/or data collection capabilities. In some embodiments, these MEA devices can comprise nanopatterned surface to support the growth or maturity of electroconductive cells or promote the growth or maturation of cells that exhibit behaviors which are more physiologically relevant than prior methods allowed (e.g., increased force generation and/or increased field potentials). Additionally, the devices provided herein allow high-throughput stimulation and data collection with a resolution not previously possible.

In some embodiments, the electrodes present on the MEA device are in contact with the cultured cells on the cell culture surface, and can be used to acquire biopotentials from the cultured cells. Leads from the electrodes can connect the electrodes to the edge of the device to transfer the signals produced by the cultured cells (e.g., biopotentials) from the cells to signal recording and/or processing electronics. In alternative embodiments, the leads which connect to the electrodes which are in contact with the cultured cells can transfer a signal (e.g., an electrical input) from a signal generator to the cultured cells. In some embodiments, the signal generator can provide an input signal which has a predefined shape and amplitude (e.g., a sine wave, a square wave or saw-tooth wave).

In some embodiments, described herein is a high-throughput screening platform for assessing peripheral neuropathy (e.g., ALS, CMT and the like) using patient hiPSC-derived neurons. In some embodiments, devices and methods as described herein incorporate innovative techniques that contribute significantly to the fields of tissue engineering and stem cell biology in both basic research and clinical settings. The biomimetic functional platform for developing physiologically advanced, ordered tissues as described herein is of practical relevance to biomedical scientists at large, since these tools can be readily disseminated to the broader biomedical community in order to aid in the advancement of their studies.

This human model system not only sheds new light on phenotypic variance in familial ALS, but it also provides a suitable test bed for therapy development studies for NMJ-related diseases, including Lambert-Eaton syndrome and myasthenia gravis, among others. Furthermore, the in vitro study of any innervated tissue can benefit from application of the nanopatterned cell sheet engineering technique, since it permits the synthesis of controlled synaptic connections between isolated populations with electrodes incorporated for real-time functional assessment. Additional applications include study of cardiac innervation by sympathetic neurons or smooth muscle innervation by the autonomic nervous system.

Integration of scalable nanofabrication technology with high-throughput electrode arrays in a convenient and electrophysiologically-compatible format constitutes a significant bioengineering advancement. The platform as described herein represents a powerful and cost-effective new tool for the continuous assessment of structurally organized neurons and skeletal muscle in culture. In some embodiments, the devices or methods described herein use hiPSCs to generate motor neurons. This permits engineering of innervated tissue from an individual patient and can be used, for example, for the generation of patient-specific drug screens for personalized medicine applications. The cross-disciplinary nature of the devices and methods described herein fosters transformative progress to the understanding of synaptic maturation and development and can pave the way to developing truly biomimetic human muscle tissue models for both in vitro and in vivo applications.

### A. Surface

As disclosed herein, the MEA device (e.g., the module) comprises a first cell growth area, a second cell growth area and an outgrowth area located there between. Such a configuration allows the cells cultured on the first cell growth area to extend cellular outgrowths, e.g., axons or other cellular extensions into and along the surface of the outgrowth area and allow these axons and cellular extensions to contact cells that are present on the second cell growth area.

(i) First Cell Growth Area

In some embodiments, the first cell growth area is configured for culture of a population of neuronal cells. In some embodiments, the first cell growth area is configured for culture of a population of neuronal cells and another cell type, such as, but not limited to, cells that support neurons, including but not limited to Schwann cells, glial cells (also referred to as neuroglia), or microglial cells. Supporting cells for neurons of the CNS can be glial cells, including oligodendrocytes, astrocytes, ependymal cells, and microglia, and supporting cells for neurons in the peripheral nervous system can be glial cells, including Schwann cells and satellite cells.

In some embodiments, the surface of the first cell growth area includes a nanotextured surface as described herein in section I.A.(v), that is configured for neuronal cell growth and maturation, including not limited to a series of nanogrooves and ridges as disclosed herein, which are oriented perpendicular to the edge or interface of where the first cell area interfaces with the axon outgrowth area. Absolute perpendicularity to the edge or interface is not required, but it helps if the neuronal cells orient in a manner that that facilitates axon growth through the axon outgrowth area. Selecting the orientation of nanotexture that facilitates such axon growth is readily achieved by one of ordinary skill in the art.

In some embodiments, the nanotextured surface of the first cell growth area is continuous with the nanotextured surface of the axon outgrowth area. In some embodiments, the nanotextured surface of the first cell growth area comprises parallel nanogrooves that are aligned parallel to the direction of the microchannels of the axon outgrowth area (as described herein in Section I.A.(iii)). In some embodiments, the nanotextured surface of the first cell growth area comprises parallel nanogrooves that are aligned perpendicular to the direction of the microchannels of the axon outgrowth area.

(ii) Second Cell Growth Area

In some embodiments, the second cell growth area is configured for culture of a population of muscle cells. In some embodiments, the second cell growth area is configured for culture of a population of muscle cells and another cell type, such as, but not limited to, cells that support muscle cells, including but not limited to satellite cells or other muscle stem cells.

In some embodiments, the surface of the second cell growth area includes a nanotextured surface as described herein in section I.A.(v), that is configured for muscle cell growth and maturation, including not limited to a series of nanogrooves and ridges as disclosed herein, which are oriented perpendicular to the edge or interface of where the second cell area interfaces with the axon outgrowth area. In some embodiments, the nanotextured surface of the second cell growth area is continuous with the nanotextured surface of the axon outgrowth area. In some embodiments, the nanotextured surface of the second cell growth area comprises parallel nanogrooves that are aligned parallel to the direction of the microchannels of the axon outgrowth area.

In some embodiments, the nanotextured surface of the second cell growth area comprises parallel nanogrooves that are aligned perpendicular to the direction of the microchannels of the axon outgrowth area (as described herein in Section I.A.(iii)). As a non-limiting example, the nanogrooves of the second cell growth area allow the long axis of a plurality skeletal myofibers to be aligned perpendicular to the long axis of neuronal axons extending through the axon outgrowth area, thus permitting properly aligned NMJ formation.

(iii) Axon Outgrowth Area

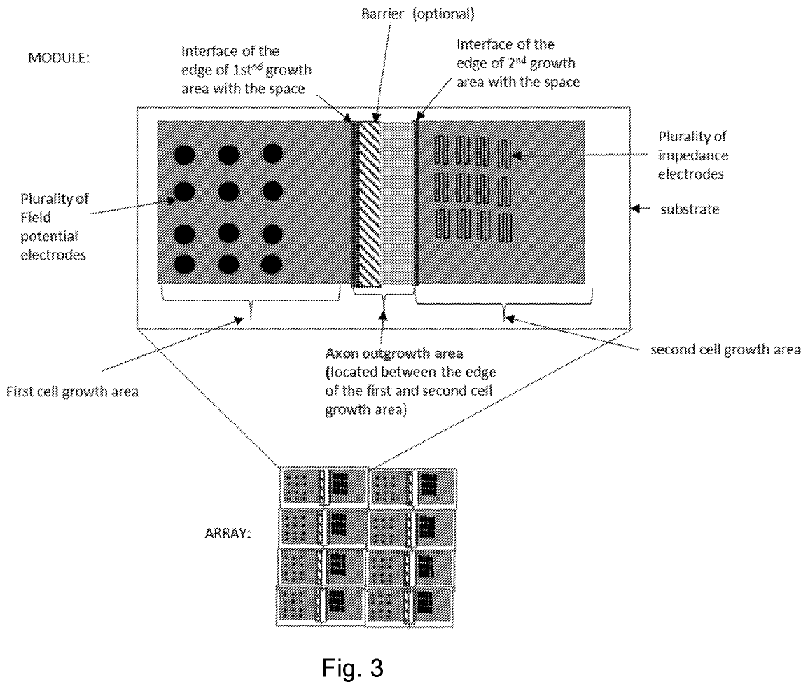

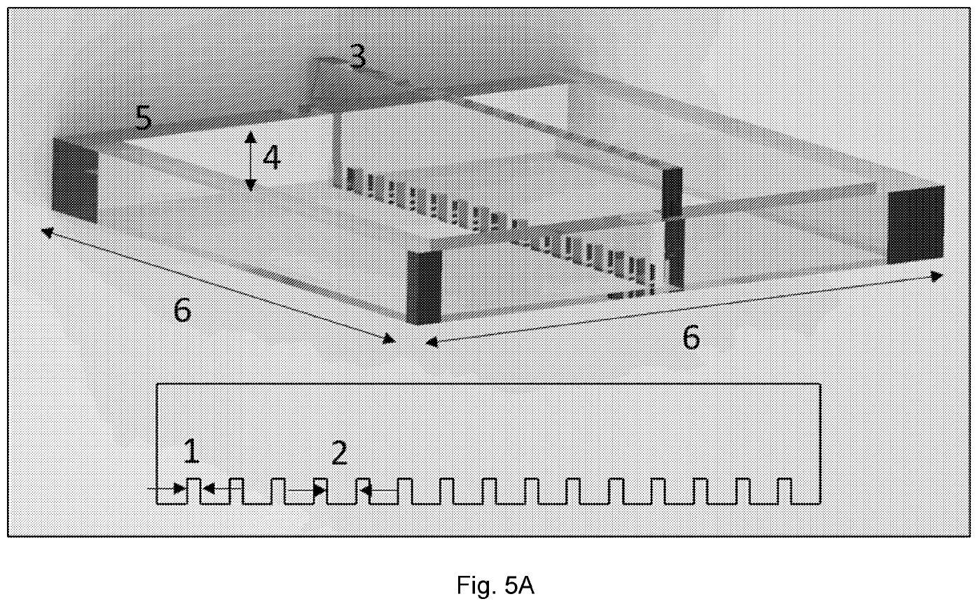

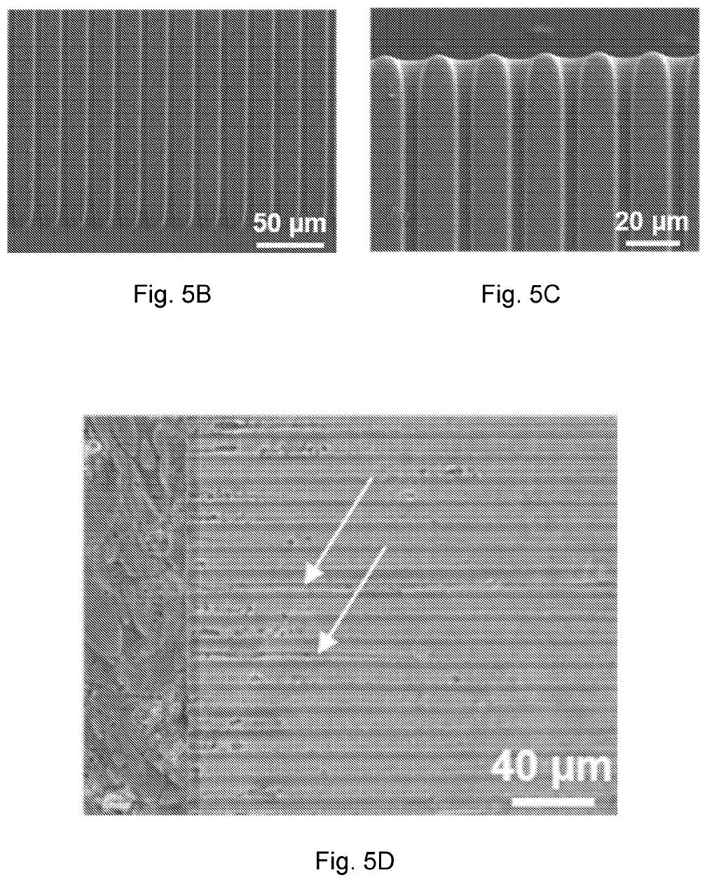

In some embodiments, the device comprises an axon outgrowth area flanked between the first cell growth area and the second cell growth area. As used herein, the phrase “axon outgrowth area” refers to the area located between the first cell growth area and the second cell growth area, e.g., in which cells can extend cellular extensions, e.g., axons, dendrites (see e.g., FIG. 1F, FIG. 3, FIG. 5D). In some embodiments, “axon outgrowth area” refers to the spatial gap or intermediate area between the area where the neurons are found and the area where the muscle cells are found. The term “axon outgrowth area” can, in the context of the area between the first and second growth areas, be used interchangeably with the terms “space”, “cell extension area”, “intermediate area”, “spatial gap” and the like.

In some embodiments, the axon outgrowth area has a width of at least 50 um between the first cell growth area and the second cell growth area—that is, the neuronal axons extend across an area of at least 50μm to reach the muscle cells on the second cell growth area. In some embodiments, the axon outgrowth area has a width of at least 50 μm, at least 75 μm, at least 100 μm, at least 150 μm, or at least 200 μm, at least 300 μm, at least 400 μm, at least 500 μm, at least 600 μm, at least 700 μm, at least 800 μm, at least 900 μm, at least 1000 μm, at least 2 mm, at least 5 mm, or at least 10 mm between the first cell growth area and the second cell growth area, or any distance between 50 μm-10 mm.

In some embodiments, the surface of the axon outgrowth area includes a nanotextured surface as described herein in section I.A.(v), that is configured for axon growth and maturation, including not limited to a series of nanogrooves and ridges as disclosed herein, which are oriented perpendicular to the edge or interface of where the first cell area interfaces with the axon outgrowth area, and perpendicular to the edge or interface of where the second cell area interfaces with the axon outgrowth area.

In some embodiments, the axon outgrowth area is configured for axons to extend from the first cell growth area to the second cell growth area. In some embodiments, the axon outgrowth area is configured for dendrites to extend from the first cell growth area to the second cell growth area. In some embodiments, the axon outgrowth area is configured for axons or dendrites to extend from the first cell growth area to the second cell growth area. In some embodiments, the axon outgrowth area is configured for cellular extensions, e.g., axons or neuronal projections, neurites, etc., to extend from the first cell growth area to the second cell growth area. In some embodiments, the cellular extensions are axons, dendrites, neuronal cell extensions, neuronal projections, neurites, or other cellular extensions.

In some embodiments, the neuronal cells on the first cell growth area extend axons through the axon outgrowth area and into the second cell growth area comprising a plurality of muscle cells. In some embodiments, the neuronal cells that extend axons through the axon outgrowth area make synaptic connections with muscle cells present on the second growth area. In some embodiments, the neuronal cell and the muscle cell are electrically connected (i.e., can send electrical signals from the neuronal cell to the muscle cell) through cellular extensions (e.g., axons, dendrites) in the axon outgrowth area.

In some embodiments, the axon outgrowth area is configured for the growth of at least a third cell type (e.g., neuronal glial cells, satellite cells, etc.). In some embodiments, the axon outgrowth area is not configured for the cell bodies of neurons (i.e., the soma comprising the nucleus). In some embodiments, the axon outgrowth area is not configured for the cell bodies of muscle cells.

In some embodiments, the axon outgrowth area comprises microchannels on the substrate. As used herein, the term “microchannel” (also referred to herein as channels or microtunnels) refers to microscale channels (i.e., wells; e.g., defined by interchannel spacing) on the surface substrate of the device, e.g., that guide the direction of the growth of axonal or cellular projections from the plurality of neurons in the first cell growth area into the second cell growth area. In some embodiments, the proximal end of the microchannel interfaces with the first cell growth area, and the distal end of the microchannel interfaces with the second cell growth area. In some embodiments, the microchannels are enclosed with a thin substrate on the open top surface (i.e., interfacing with the ridges but not extending into the channels) to form microtunnels.

In some embodiments, the axon outgrowth area is configured such that the microchannels are of a sufficient size to allow the growth of cell extensions (e.g., axons, dendrites), optionally with the inclusion of support cells (e.g., myelinating cells such as Schwann cells or oligodendrocytes). In some embodiments, the axon outgrowth area is configured such that the microchannels are not of a sufficient size (e.g., too small or limiting) to allow the growth or migration of certain cell bodies (e.g., neuronal cells) or certain cells (e.g., muscle cells). That is the soma of the neuronal cells and muscle cells remain in the first cell growth area and second cell growth area, respectively.

In some embodiments, the microchannels comprise a series of parallel channels with a proximal and distal end, wherein the proximal end of the microchannels interfaces with the first cell growth area, and the distal end of the microchannels interfaces with the second cell growth area.

In some embodiments, the microchannel depth is between 50 nm and 5000 nm (i.e., 5 μm), (see e.g., FIG. 1B-1C, FIG. 1F, FIG. 5A-5D). Accordingly, in some embodiments, the cell culturing surface of the axon outgrowth area can comprise a substantially parallel array of microchannels which have a microchannel depth of about between about 10 nm-10 μm, or between about 50 nm-5000 nm, or between about 100 nm-1000 nm, or between about 10 nm-600 nm, or between about 300 nm-900 nm, or between about 400 nm- 800 nm, or between about 500 nm- 700 nm, or between about 550 nm-650 nm. In some embodiments, the microchannel depth is approximately 3000 nm (i.e., 3 μm). In some embodiments, the microchannel depth is between 100 nm and 600 nm.

In some embodiments, the interchannel spacing is between 1 μm and 150 μm (see e.g., FIG. 1B-1C, FIG. IF, FIG. 5A-5D). Accordingly, in some embodiments, the cell culturing surface of the axon outgrowth area can comprise a substantially parallel array of microchannels which have a microchannel spacing of about between about 1μm -10 μm, or between about 10 μm -50 μm, or between about 50 μm-100 μm, or between about 100 μm-150 μm, or between about 5 μm -10 μm, or between about 10 μm -20 μm, or between about 15 μm -25 μm. In some embodiments, the interchannel spacing is approximately 20 μm.

In some embodiments, the width of the microchannels is between the range of about 1 μm-50 μ, 1 μm-10 or between about 10 μm-50 μm, or between about 5 μm-10 μm, or between about 10 μm-20 μm, or between about 15 μm -25 μm, or between about 20 μm-30 μm, or between about 30 μm-40 μm, or between about 40 μm -50 μm. In some embodiments, the interchannel spacing is approximately 20 μm. In some embodiments, the microchannel width is approximately 10 μm.

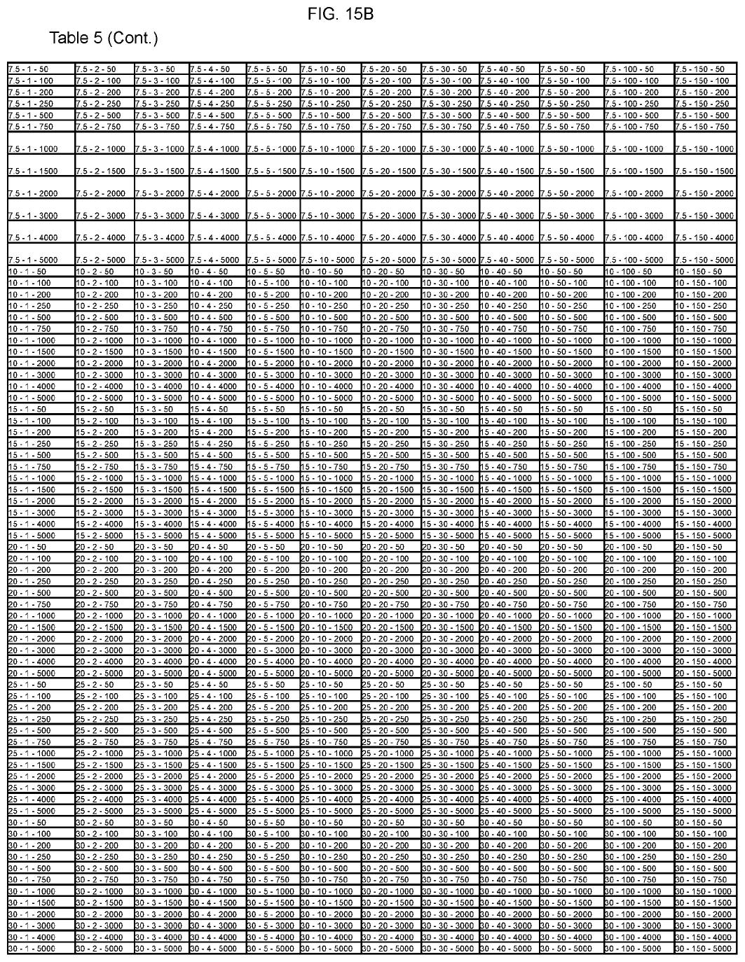

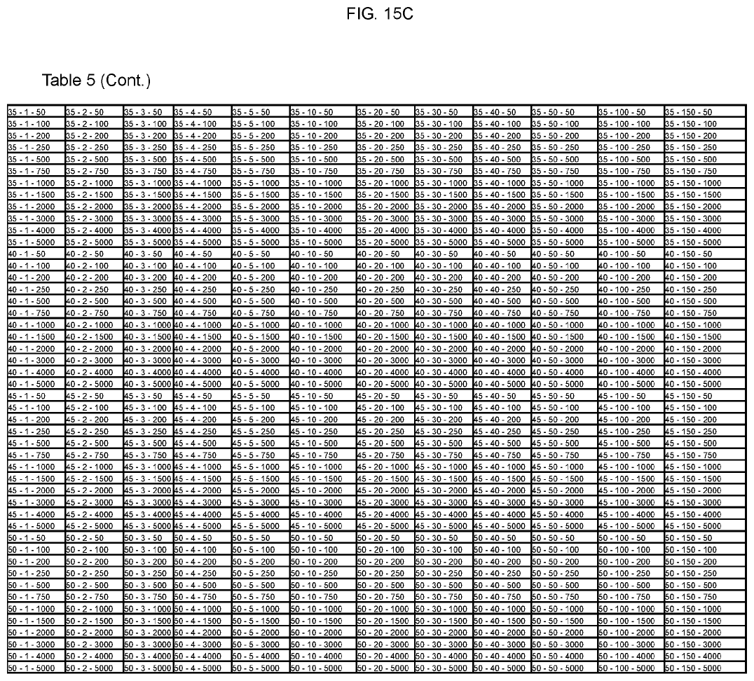

In some embodiments, the microchannels can have the dimensions of X-Y-Z, where X is the width of the microchannel, Y is the interchannel spacing, and Z is the depth of the microchannel In some embodiments, X is selected from any of 1 μm, 2 μm, 3 μm, 4 μm, 5 μm, 7.5 μm, 10 μm, 15 μm , 20 μm, 25 μm, 30 μm, 35 μm, 40 μm, 45 μm, 50 μm; Y is selected from any of 1 μm, 2 μm, 3 μm, 4 μm, 5 μm, 10 μm, 20 μm, 30 μm, 50 μm, 100 μm, 150 μm; and Z is selected from any of 50 nm, 100 nm, 200 nm, 250 nm, 500 nm, 750 nm, 1000 nm, 1500 nm, 2000 nm, 3000 nm, 4000 nm, 5000 nm. Exemplary combinations of X-Y-Z (e.g., for microchannels of the surface substrate of the axon outgrowth area) are shown in Table 5 in FIGS. 15A-15C.

In some embodiments, the microchannels extend the entire distance of the axon outgrowth area, and as such are the width of the axon outgrowth area (i.e., the distance of the space in between the first cell growth area and the second cell growth). Accordingly, in some embodiments, the length of the microchannels is at least 50 μm. In some embodiments, the axon outgrowth area has a length of at least 50 μm, at least 75 μm, at least 100 μm, at least 150 μm, or at least 200 μm, at least 300 μm, at least 400 μm, at least 500 μm, at least 600 μm, at least 700 μm, at least 800 μm, at least 900 μm, at least 1000 μm, at least 2 mm, at least 5 mm, or at least 10 mm between the first cell growth area and the second cell growth area, or any distance between 50 μm-10 mm. In some embodiments, the dimensions of the microchannels (e.g., as described in FIGS. 15A-15C, Table 5) can be combined with any width of the axon outgrowth area as described herein.

(iv) Additional cell growth areas

In some embodiments, the device further comprises at least one cell growth area in addition to the first cell growth area and the second cell growth area, i.e., at least one additional cell growth area. As a non-limiting examples, the device can further comprise 1 additional cell growth area, 2 additional cell growth areas, 3 additional cell growth areas, 4 additional cell growth areas, or 5 additional cell growth areas. In some embodiments, the term “cell surface area” is used interchangeably with “cell growth area”.

In some embodiments, the device further comprises a third cell surface area. In some embodiments, an edge of the third cell surface area interfaces with a proximal edge of the first cell growth area and the axon outgrowth area. That is, the device comprises in the following configuration, a first cell growth area, an additional or third cell growth area, an axon outgrowth area and a second cell growth area, such that the additional cell growth area is located between the first cell growth area and axon outgrowth area so the axon extensions grow through an additional cell growth area comprising supporting cells after their extension from the neurons on the first cell growth area. In some embodiments, an edge of the third cell growth area interfaces with a distal edge of the second cell growth surface and the axon outgrowth area. That is, the device comprises in the following configuration, a first cell growth area, an axon outgrowth area, an additional or third cell growth area, and a second cell growth area, such that the additional cell growth area is located between the axon outgrowth area and the second cell growth area, so the axon extensions grow through an additional cell growth area comprising supporting cells prior to reaching the muscle cells on the second cell growth area.

In some embodiments, the additional cell growth area is configured for culture of a population of glial cells, satellite cells, or another type of support cell type for neuronal or muscle cells.

In some embodiments, the additional (i.e., third) cell growth area is configured for culture of an additional population of neuronal cells. Accordingly, the additional cell growth area interfaces with an additional (i.e. second) axon outgrowth area that interfaces with the second cell growth area comprising muscle cells. The neuronal cells in the third cell growth area can be a different type or genotype than the neuronal cells in the first cell growth area.

In some embodiments, the surface of the additional cell growth area includes a nanotextured surface as described herein in section I.A.(v), that is configured for cell growth and maturation, including not limited to a series of nanogrooves and ridges as disclosed herein, which are oriented perpendicular to the edge or interface of where the additional cell area interfaces with at least one other cell growth area.

(v) Nanotextured Surface

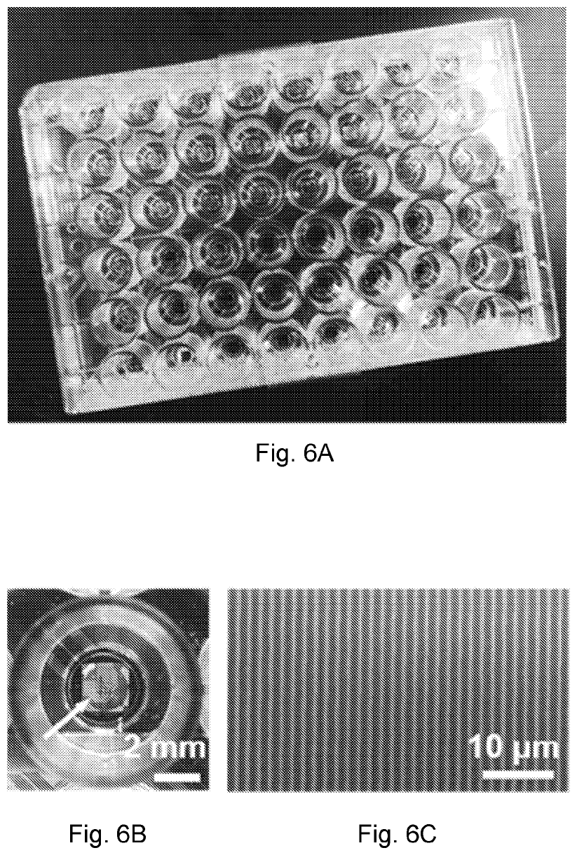

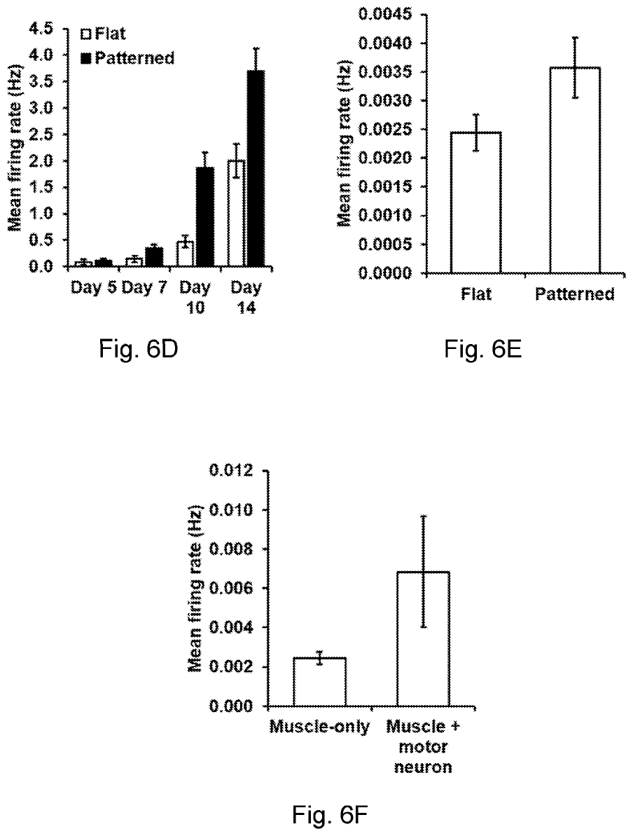

In some embodiments, the first cell growth area, or second cell growth surface, or both, or axon outgrowth area, or additional cell growth area, or any combination thereof, has a substantially smooth surface. In other embodiments the first cell growth area, or second cell growth, or axon outgrowth area, or additional cell growth area, or any combination thereof, can be nanotextured, including an array of substantially parallel grooves and ridges of nanometer widths. In some embodiments, the MEA device comprises parallel nanogrooves, which have been shown to enhance skeletal muscle structure and function as well as motor neuron axon guidance and spontaneous firing rates (see e.g., FIG. 6A-6F). In some embodiments, the nanopatterned surface provides anisotropic cues that promote improved levels of maturation of neuronal cell types, motor neurons and myocytes.

In some embodiments, the nanopattern on the first cell growth area is different to the nanopattern on the second cell growth area. For example, in some embodiments the nanopattern on the first cell growth area is configured for optimal growth and maturation of neuronal cells, whereas the nanopattern on the second cell growth area is configured for optimal growth and maturation of muscle cells and myocytes. In some embodiments, the nanopattern on the first cell growth area is the same as the nanopattern on the second cell growth area.

In some embodiments, the compartments of the MEA device (e.g., the first growth area, the second growth area, the axon outgrowth area, and/or an additional cell growth area) comprise the same pattern and type of nanogrooves. In some embodiments, the nanogrooves are continuous between the compartments of the MEA device, e.g., with parallel nanogrooves, each nanogroove continuing in one line from the first growth area through the axon outgrowth area into the second growth area. In some embodiments, the compartments of the MEA device comprise the same pattern and type of nanogrooves, but the nanogrooves are non-continuous between at least two compartments. In some embodiments, at least two compartments of the MEA device comprise substantially different patterns and/or types of nanogrooves. In some embodiments, at least one compartment of the MEA device lacks nanogrooves or has a substantially smooth surface.

Accordingly, in some embodiments, these MEA devices can comprise nanopatterned surface to support the growth or maturity of electroconductive cells or to promote the growth or maturation of cells that exhibit behaviors which are more physiologically relevant than prior methods allowed (e.g., increased force generation and/or increased field potentials).

In some embodiments, any one or more of the first, or second cell growth area can comprise nanotopography, that is, a nanotopographic or nanotextured surface, that can be used to enhance both muscle and neuron maturation (see e.g., Yang et al. Biomaterials 2014, 35:1478-86; Tonazzini et al. Biomaterials 2013, 34:6027-36; Yang et al. ACS Appl Mater Interfaces 2013, 5:10529-40; Yang et al. Macromol Biosci. 2015, 15:1348-56; which are incorporated herein by reference in their entireties) thereby leading to improved synaptic development of neurons and greater stratification of neuromuscular diseases, including CMT and ALS disease phenotypes.

It has been previously demonstrated that nanotextured grooves and ridges in a substrate promoted anisotropic arrangement of cardiomyocytes isolated from adult tissue (see e.g., Kim et al., entitled “Nanoscale cues regulate the structure and function of macroscopic cardiac tissue constructs”, PNAS (2010) 107 (2); 565-570, and in U.S. Patent Application 61/620,301, filed on Apr. 4, 2012, and International Patent Publication WO 2013/151755, and U.S. Pat. No. 9,994,812; each of which are incorporated herein in their entirety by reference). Accordingly, in alternative embodiments, the cell culturing surface of the MEA device can comprise a nanotextured platform comprising a substantially parallel array of grooves and ridges which have a groove depth (or ridge height) of between about 10 nm-10 μm, or between about 10 nm-1000 nm, or between about 50 nm-500 nm, or between about 10 nm-600 nm, or between about 300 nm- 900 nm, or between about 400 nm- 800 nm, or between about 500 nm-700 nm, or between about 550 nm- 650 nm.

In some embodiments, the width of the grooves is between the range of about 1 μm-50 μm, or in the range of about 50 nm-10 μm, or in the range of about 200 nm-1000 nm, or in the range of about 5 nm-1000 nm, or in the range of about 700 nm-900 nm, or in the range of about 750nm-850 nm where ridges, between the grooves, have a width in the range of about 50 nm-10 μm, or in the range of about 400 nm-1000 nm, or in the range of about 200 nm-1000 nm, or in the range of about 5 nm-1000 nm, or in the range of about 700 nm-900 nm, or in the range of about 750nm-850 nm. In some embodiments, the width of the groove and/or ridge is between the range of 200 nm-800 nm, and the depth of the groove (or height of the ridge) is between about 20 nm-100 nm.

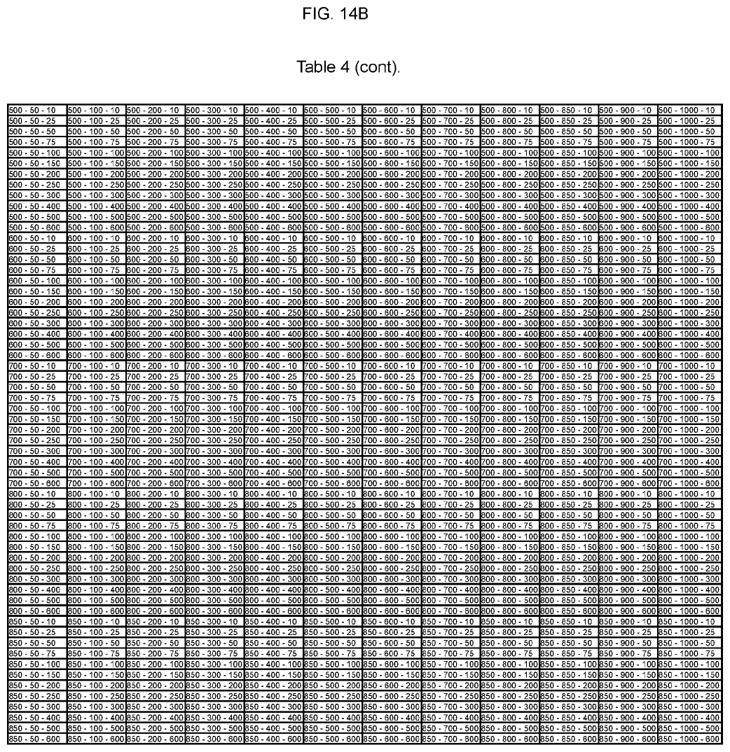

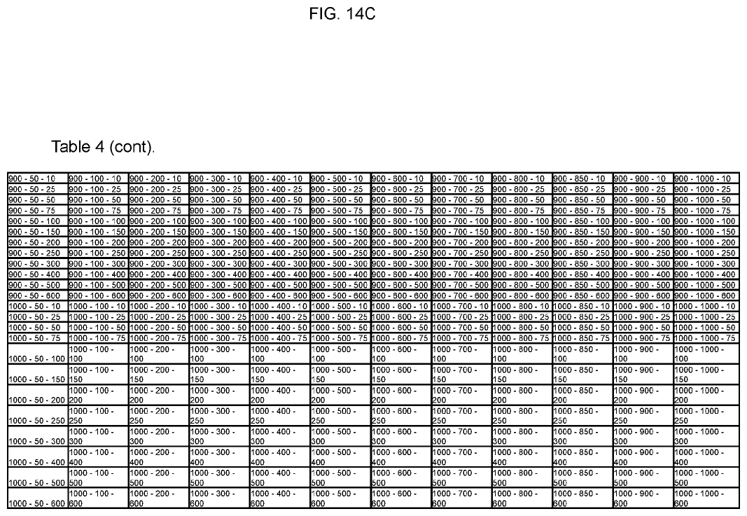

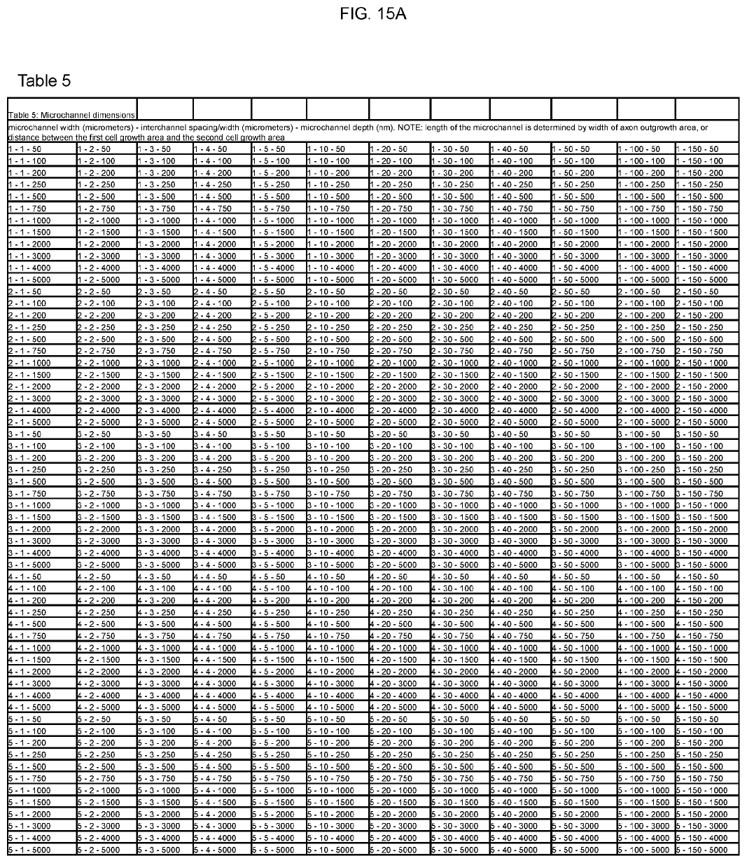

In some embodiments, the nanotextured surface can have the dimensions of X-Y-Z, where X is the width of the groove, Y is the width of the ridge, and Z is the depth of the groove. In some embodiments, X is selected from any of 50 nm, 100 nm, 200 nm, 300 nm, 400 nm, 500 nm, 600 nm, 700 nm, 800 nm, 850 nm, 900 nm, 1000 nm; Y is selected from any of 50 nm, 100 nm, 200 nm, 300 nm, 400 nm, 500 nm, 600 nm, 700 nm, 800 nm, 850 nm, 900 nm, 1000 nm; and Z is selected from any of 10 nm, 25 nm, 50 nm, 75 nm, 100 nm, 150 nm, 200 nm, 250 nm, 300 nm, 400 nm, 500 nm, 600 nm. Exemplary combinations of X-Y-Z for the nanogrooves (e.g., of the first cell growth area, the second cell growth area, the axon outgrowth area, and/or additional cell growth areas) are shown in Table 4 in FIGS. 14A-14C.

Methods of fabricating a cell culturing layer with nanotextured surfaces for use as a MEA device are disclosed, e.g., in the PCT Application No. PCT/US2013/032237 entitled “Systems and Methods for Engineering Muscle Tissue” by Kim et al., filed on Mar. 15, 2013, published as WO 2013/151755 and patented as U.S. Pat. No. 9,994,812; each of which is incorporated by reference herein in its entirety.

In some embodiments, the electroconductive cells can form a monolayer on the MEA device as disclosed herein with anisotropic and polarized cell arrangement in the direction of the nanotextures. This can be evidenced by, for example, a high spindle shape factor or major axis of cells aligned in parallel to nanotextured arrays, as detected for example, using fluorescent or other microscopy.

In some embodiments, the substantially parallel grooves and ridges present on the microelectrode (MEA) array can be fabricated over a large surface area with high fidelity and, in some embodiments, can cover a surface area greater than (>1 cm²). In some embodiments, the array of parallel grooves and ridges has a precision of texture of at least 90% fidelity, as evidenced by atomic force microscopy, and electron microscopy.

In accordance with the various embodiments, the nanometer and micrometer scale features can be configured and arranged on the cell culturing surface to encourage organization of cells and tissue during culturing and to test cell and tissue function. In accordance with some embodiments, the width, depth and/or pitch (e.g., spacing between nanogrooves) of the nanogrooves can be substantially uniform over the length of each nanogroove. In accordance with some embodiments, the width, depth and/or pitch (e.g., spacing between nanogrooves) of the nanogrooves can be substantially non-uniform (e.g., irregular) over the length of each nanogroove. In accordance with some embodiments, two or more of the nanogrooves can extend substantially parallel over at least a portion of the cell culturing surface. In accordance with some embodiments, the distance between two or more of the nanogrooves can vary over at least a portion of the cell culturing surface and can intersect one or more times. In accordance with some embodiments, one or more of the nanogrooves can be curved and the curve can be regular (e.g., having a constant radius of curvature) or irregular (e.g., having different radii of curvature over the length of the nanogroove).

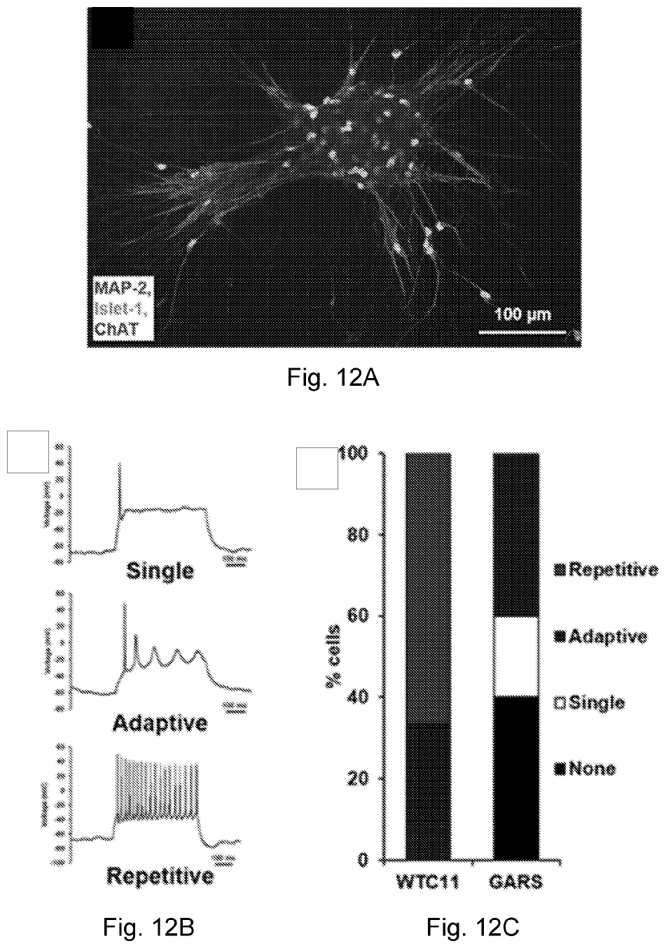

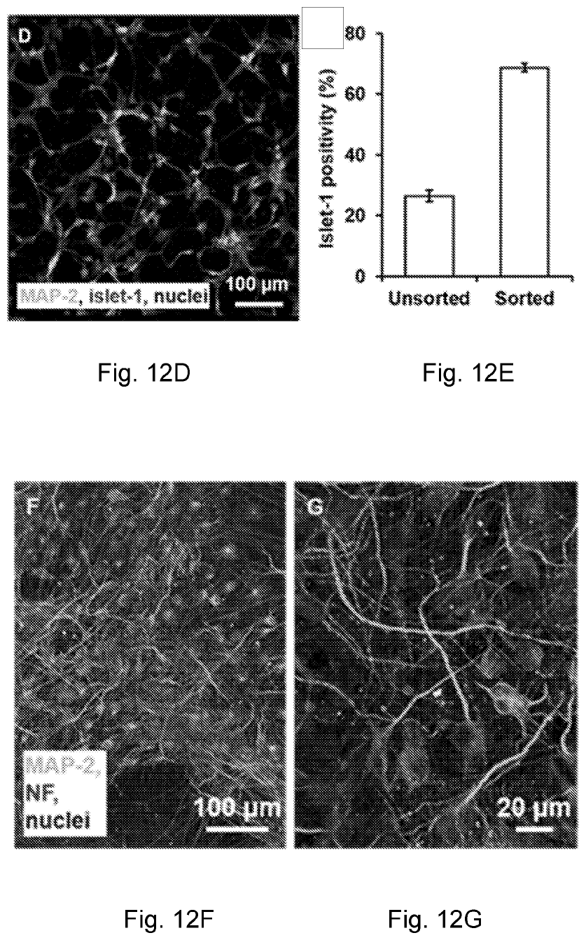

In some embodiments, the nanosurface used is described in U.S. Pat. No.9,994,812, which is incorporated herein by reference in its entirety. In some embodiments, the nanogrooves can be formed in concentric circles such as shown in FIG. 12A of PCT/US2014/028530 entitled “Device and methods comprising electrode arrays for electroconductive cells”, filed on Mar. 14, 2014, published as WO 2014/144219 and corresponding US publication US 2016/0017268; each of which is incorporated by reference herein in its entirety. In accordance with some embodiments, the width and depth of the grooves can be different, resulting in a different amount of cell protrusion into the nanogroove. For example, FIG. 12B of US 2016/0017268 shows primary cardiac cell protrusion into a 400-nm-wide groove (left) and an 800-nm-wide groove (right). In accordance with some embodiments, the spacing between nanogrooves and/or the width of each nanogroove can vary over the cell culturing surface. For example, FIG. 12C of US 2016/0017268 shows a fibroblast response to variable ridge pattern arrays with graded spacing between nanogrooves (e.g., increasing left to right). FIG. 12D of US 2016/0017268 shows a fibroblast response to regularly spaced topographic nanogroove pattern arrays. Similarly, the depth of the nanogrooves can vary over the length of the groove and/or be different from one nanogroove to an adjacent nanogroove.

Further, the MEA devices can be configured with compound configurations of nanogrooves and other nanometer sized features that can include different features in different regions or areas of the cell culturing surface. For example, one or more straight and uniform nanogrooves can extend along one area and an adjacent area can include a curved, a spiral and/or an irregularly configured portion.

According to some embodiments, the array of electrodes can be positioned or arranged in a predefined configuration with respect to the nanofeatures applied to the cell culturing surface. For example, the nanofeatures (e.g., nanogrooves or ridges) can be configured to cause the cells and/or tissue to align along a first axis and the array of electrodes can be positioned to contact the cells and/or tissue at predefined locations with respect to the axis. In another example, the cell culturing surface can include a region having a different or an irregular configuration of nanofeatures and the electrodes can be strategically placed with respect to those nanofeatures.

As used, the term “irregular” when used in reference to grooves or nanogrooves refers to configurations other than straight, uniform, and parallel sets of grooves or nanogrooves. In some embodiments, irregular grooves (or nanogrooves) are those with increased anisotropy relative to straight, parallel, and uniform grooves (or nanogrooves). In some embodiments, the irregular grooves (or nanogrooves) can comprise curved grooves, intersection grooves, spiral patterned grooves, semi-randomly configured grooves, and/or randomly configured grooves. In some embodiments, a portion of a cell culture surface comprises irregular grooves (or nanogrooves) while another portion comprises regular grooves (or nanogrooves) (e.g., straight, uniform, and parallel).

These irregular nanogrooves can permit the culture and/or study of, e.g., cardiomyocytes with arrhythmias and/or cardiomyocytes which are not properly aligned. Misaligned cardiomyocytes can be found in the cardiac tissue of subjects who have experienced a myocardial infarction, the cardiac tissue of subject with congenital heart defects, and/or in scar tissue of the heart. Such devices can be used, as described herein, to, by the way of non-limiting example, screen for anti-arrhythmic agents or agents to treat cardiac scar tissue and/or heart diseases.

(vi) Chamber

In accordance with the various embodiments described herein, the MEA preferably includes a chamber that maintains cell/tissue viability by immersion in media. The chamber can be any structure that maintains the cells/tissue in the media. In accordance with some embodiments, the MEA can include chamber walls that encircle the MEA and encourage cells to culture over some of the electrodes in the array. In some embodiments, one or more of the chamber walls of the MEA device is removable. In some embodiments, one or more chamber walls that interface with the axon extension area can be removed, with one or more of the chamber walls that encircle or around the circumference of the MEA are not removed.

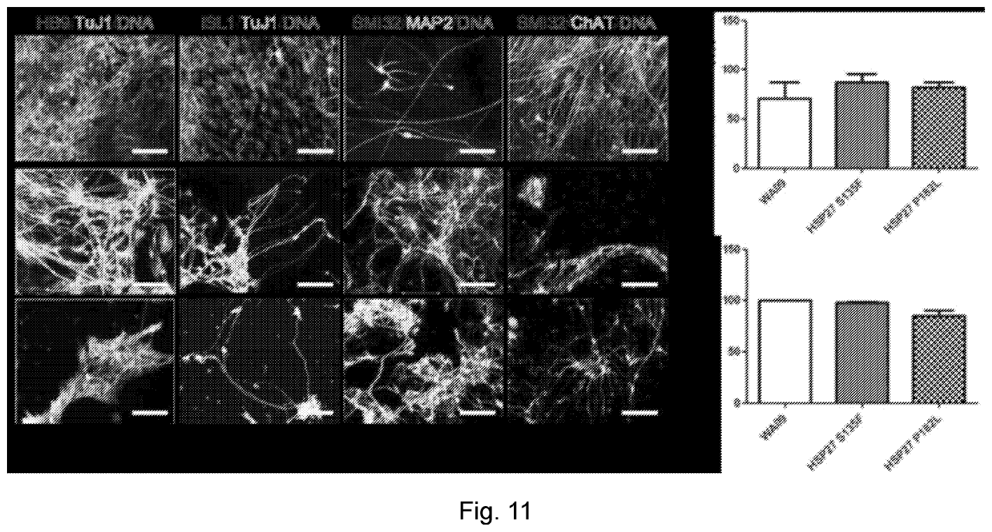

In some embodiments, the module (e.g., comprising first cell growth area, axon outgrowth area, second cell growth area, and electrode surface) is contained within a walled chamber constructed of a biocompatible substrate, as described herein; i.e., the walled chamber comprises the module as described herein (see e.g., FIG. 5A). In some embodiments, the walls of the chamber directly interface with the surface substrate, such that the chamber is watertight and can contain cell growth media. The chamber can define any shape, from circular (as shown in FIGS. 11A and 24 of US 2016/0017268; see e.g., FIG. 5A) or oval to regular and irregular polygons. In accordance with some embodiments, the shape of the chamber can be defined by the intended cell and tissue development or to encourage a predefined cell/tissue organization.

In some embodiments, the height of the chamber walls is 250 μm. As a non-limiting example, the chamber walls are at least 100 μm, at least 150 μm, at least 200 μm, at least 250 μm, at least 300 μm, at least 350 μm, at least 400 μm, at least 450 μm, or at least 500 μm tall, at least 1 mm, at least 2 mm, at least 3 mm, at least 4 mm, at least 5 mm, or at least 10 mm. In some embodiments, the width of the chamber walls is 2 mm to 3 mm. As a non-limiting example, the chamber walls are at least 0.1 mm (i.e., 100 μm) at least 0.5 mm, at least 1 mm, at least 2 mm, at least 3 mm, at least 4 mm, at least 5 mm, at least 6 mm, at least 7 mm, at least 8 mm, at least 9 mm, at least 10 mm wide.

In some embodiments, the length of the chamber walls is 20 mm. As a non-limiting example, the barrier is at least 5 mm, is at least 10 mm, at least 20 mm, at least 30 mm, at least 40 mm, or at least 50 mm long. In some embodiments, the chamber walls form a square, with right angles and four equally long sides.

(vii) Barrier

In some embodiments, the device as described herein further comprises at least one barrier located between the first cell growth area and the second cell growth area. As a non-limiting example, the device further comprises 1 barrier, 2 barriers, 3 barriers, 4 barriers, or even 5 barriers in between the first cell growth area and the second cell growth area. In some embodiments, the barrier is configured to separate a plurality of cell bodies of cells located on the first cell growth area from cells located on the second growth area. In some embodiments, a chamber wall can serve as a barrier located between the first cell growth area and the second cell growth area.

In some embodiments, the barrier is located at the interface between the first cell growth area and the axon outgrowth area. In some embodiments, the barrier is located at the interface between the second cell growth area and the axon outgrowth area. In some embodiments, the device further comprises at least one barrier between the at least one additional growth area and the first cell growth, axon outgrowth area, and/or second cell growth area. In some embodiments, a barrier extends the entire width of the axon outgrowth area and/or the entire length of the axon outgrowth area.

In some embodiments, the barrier is located within the axon outgrowth area, and the barrier is configured to separate a plurality of cell bodies of cells located on the first cell growth area from cells located on the second growth area. In some embodiments, the barrier is configured to allow axons from cells located on the surface of the first cell growth area to extend into the axon outgrowth area.

In some embodiments, the barrier is configured such that the barrier comprises voids that are of a sufficient size to allow the growth of cell extensions (e.g., axons, dendrites), optionally with the inclusion of support cells (e.g., myelinating cells such as Schwann cells or oligodendrocytes). In some embodiments, the barrier is configured such that the barrier voids are not of a sufficient size (e.g., too small or limiting) to allow the growth of certain cell bodies (e.g., neuronal cells) or certain cells (e.g., muscle cells). In other words, while the cell bodies of the first cell type and the second cell type are spatially separated by the barrier, the barrier is configured to permit the axons or neurite outgrowths to extend beyond the barrier such that the neurons and muscle cells are connected via axon and dendritic outgrowths (i.e., the cells touch) and they are connected electrically.

In some embodiments, the barrier is configured to interface with the surface (e.g., of the axon outgrowth area), which is optionally a surface comprising microchannels, a nanopatterned surface or a substantially smooth surface. In some embodiments, where the surface or the axon outgrowth area comprises microchannels, the barrier extends to the top of the microchannel ridges but not into the wells of the microchannels, thereby allowing axons or other cell extensions to extend beyond the barrier by passing through the wells of the microchannels in the surface of the axon outgrowth area. In some embodiment, where the barrier interfaces with a substantially smooth surface, e.g., absence of nanotextures or microchannels, the edge of the barrier interfacing with the surface comprises a series of voids. In some embodiments, the voids of the barrier are referred to herein as “microchannels of the barrier” or “microtunnels of the barrier”. In some embodiments, the voids of the barrier are parallel and/or aligned with the microchannels of the surface substrate as described herein.

In some embodiments, the voids of the barrier are at least 5 μm wide. In some embodiments, the voids of the barrier are from at least 1 μm wide to at least 50 μm wide. As a non-limiting example, the voids of the barrier are at least 1 μm, at least 2 μm, at least 3 μm, at least 4 μm, at least 5 μm, at least 6 μm, at least 7 μm, at least 8 μm, at least 9 μm, at least 10 μm, at least 15 μm, at least 20 μm, at least 25 μm, at least 30 μm, at least 35 μm, at least 40 μm, at least 45 μm, or at least 50 μm wide. In some embodiments, the voids of the barrier are of the same width as the microchannels of the surface substrate as described herein. In some embodiments, the voids of the barrier are a substantially different width than the microchannels of the surface substrate as described herein.

In some embodiments, the inter-void spacing of the barrier is at least 20 μm. As a non-limiting example, the inter-void spacing of the barrier is at least 1 μm, at least 2 μm, at least 3 μm, at least 4 μm, at least 5 μm, at least 6 μm, at least 7 μm, at least 8 μm, at least 9 μm, at least 10 μm, at least 11 μm, at least 12 μm, at least 13 μm, at least 14 μm, at least 15 μm, at least 16 μm, at least 17 μm, at least 18 μm, at least 19 μm, at least 20 μm, at least 25 μm, at least 30 μm, at least 35 μm, at least 40 μm, at least 45 μm, or at least 50 μm. In some embodiments, the spacing in between the voids of the barrier is of the same width as the spacing in between the microchannels of the surface substrate as described herein. In some embodiments, the spacing in between the voids of the barrier is of a substantially different width as the spacing in between the microchannels of the surface substrate as described herein.

In some embodiments, the voids of the barrier are at least 5 μm high. As a non-limiting example, the voids of the barrier at least 0.5 μm, at least 1μm, at least 2μm, at least 3 μm, at least 4 μm, at least 5 μm, at least 6 μm, at least 7 μm, at least 8 μm, at least 9 μm, at least 10 μm, at least 15 μm, at least 20 μm, at least 30 μm, or at least 40 μm, at least 50 μm high, at least 100 μm high, or at least 150 μm high. In some embodiments, the voids of the barrier are of the same height or depth as the microchannels of the surface substrate as described herein. In some embodiments, the voids of the barrier are a substantially different height or depth than the microchannels of the surface substrate as described herein.

In some embodiments, the voids of the barrier can have the dimensions of X-Y-Z, where X is the width of the void of the barrier, Y is the inter-void spacing, and Z is the depth of the void. In some embodiments, X is selected from any of 1 μm, 2 μm, 3 μm, 4 μm, 5 μm, 7.5 μm, 10 μm, 15 μm, 20 μm, 25 μm, 30 μm, 35 μm, 40 μm, 45 μm, 50 μm. Y is selected from any of 1 μm, 2 μm, 3 μm, 4 μm, 5 μm, 10 μm, 20 μm, 30 μm, 40 μm, 50 μm, 100 μm, 150 μm; and Z is selected from any of 50 nm, 100 nm, 200 nm, 250 nm, 500 nm, 750 nm, 1000 nm, 1500 nm, 2000 nm, 3000 nm, 4000 nm, 5000 nm. Exemplary combinations of X-Y-Z (e.g., for voids of the barrier) are shown in Table 5 in FIGS. 15A-15C.

In some embodiments, the barrier is configured to directly interface (i.e., physically interacts) with the microchannel ridges of the surface substrate, and the surface-interfacing edge of the barrier is smooth, i.e., contains no voids, such that the only remaining space for the growth of cell extensions (e.g., axons, dendrites) is within the microchannels of the surface substrate.

In some embodiments, the barrier is configured to indirectly interface (i.e., does not physically interact) with surface substrate, and the surface-interfacing edge of the barrier is smooth, i.e., contains no voids, such that the only remaining space for the growth of cell extensions (e.g., axons, dendrites) is within any microchannels surface substrate and/or any space in between the surface-interfacing edge of the barrier and the surface substrate. As a non-limiting example, the space between the surface-interfacing edge of the barrier and the surface substrate can be at least 0.1 μm, at least 0.5 μm, at least 1.0 μm, at least 2.0 μm, or at least 5 μm.

In some embodiments, the barrier is designed to be a non-removable physical barrier. In some embodiments, the barrier is designed to be a removable physical barrier. In some embodiments, the barrier can be removed at some point after the axons have extended into the axon outgrowth area. In some embodiments, the barrier can be removed at some point after the axons have extended into the second cell growth area. In some embodiments, the barrier can be removed at some point after the axons have formed synaptic connections with cells in the second cell growth area. In some embodiments, the barrier can be removed at some point after glial cells (e.g., Schwann cells, oligodendrocytes) have grown on or around the axons.

In some embodiments, the barrier is the same width as the axon outgrowth area, i.e., extends from the first cell growth area to the second cell growth area. In some embodiments, the barrier has a thickness of at least 1 μm, at least 2 μm, at least 3 μm, at least 4 μm, at least 5 μm, at least 6 μm, at least 7 μm, at least 8 μm, at least 9 μm, at least 10 μm, at least 11 μm, at least 12 μm, at least 13 μm, at least 14 μm, at least 15 μm, at least 16 μm, at least 17 μm, at least 18 μm, at least 19 μm, at least 20 μm, at least 25 μm, at least 30 μm, at least 35 μm, at least 40 μm, at least 45 μm, at least 50 μm, at least 55 μm, at least 60 μm, at least 65 μm, at least 70 μm, at least 75 μm, at least 80 μm, at least 85 μm, at least 90 μm, at least 95 μm, at least 100 μm, at least 150 μm, at least 200 μm, at least 300 p.m, at least 400 p.m, at least 500 μm, at least 600 μm, at least 700 μm, at least 800 μm, at least 900 p.m, at least 1000 p.m (i.e., 1 mm), at least 2 mm, at least 5 mm, or at least 10 mm. In some embodiments, the barrier is a smaller width than the axon outgrowth area. In some embodiments, the barrier is wider than the axon outgrowth area. In some embodiments, the barrier is at least 1 mm wide.

In some embodiments, the barrier is the same length as the length of the axon outgrowth area or chamber. As a non-limiting example, the barrier is at least 10 mm, at least 20 mm, at least 30 mm, at least 40 mm, or at least 50 mm long. In some embodiments, the height of barrier is the same or greater height than the height of the device (e.g., chamber wall). As a non-limiting example, the barrier is at least 0.1 mm, at least 0.25 mm, at least 0.5 mm, at least 1 mm, at least 2 mm, at least 3 mm, at least 4 mm, at least 5 mm, at least 6 mm, at least 7 mm, at least 8 mm, at least 9 mm, or at least 10 mm tall. In some embodiments, the barrier interfaces with the outer walls of the device, e.g., through grooves or overhangs (see e.g., FIG. 5A). In some embodiments, the barrier is constructed of a biocompatible substrate, as described herein.

(viii) Substrate

In some embodiments, the MEA device comprising the electrode arrays can be made of a polymer known to one of ordinary skill in the art. As used herein, “base substrate” refers to the material comprising the electrode arrays, e.g., attached to which can be a surface substrate, i.e., a cell-culturing surface. In some embodiments, the MEA device can be fabricated from glass or any other substrate comprising silica; e.g., the MEA device can comprise a glass slide. In some embodiments, the MEA device can be nanofabricated from scalable biocompatible polymers, such as but not limited to polyethene glycol (PEG), polyethene glycol-gelatin methacrylate (PEG-GelMA) and chemical variants thereof and hydrogel arrays. Others include, for example, but not limited to polyurethane acrylate (PUA), PLGA, glycidyl methacrylate (GMA), or poly(methyl methacrylate) (PMMA), polydimethylsiloxane (PDMS), or any combination thereof. Further, in some embodiments, the MEA device can be optically transparent, so as to facilitate observation of the cells cultured on the MEA cell culturing surface. Optical transparency can be achieved for many MEA device materials by making the structure sufficiently thin as to permit light to transmit.

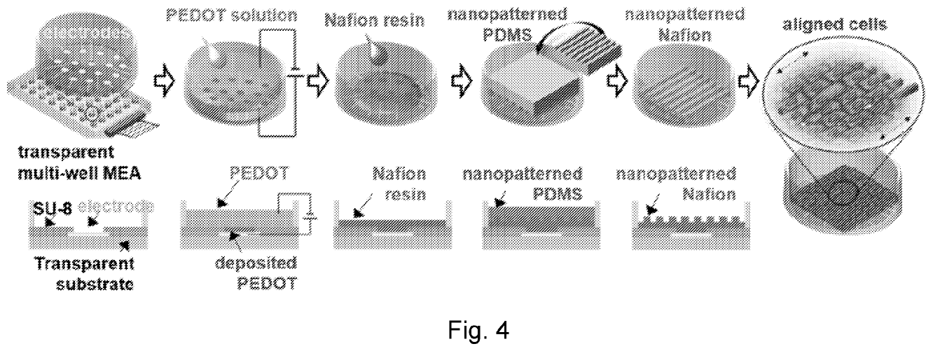

In some embodiments, the MEA device can include a biocompatible and/or biodegradable substrate or layer. As used herein, the term “surface substrate”, “cell-culturing surface”, “biocompatible layer” or “substrate layer” refers to the material of the MEA device to which the cells are directly attached; the surface substrate is attached to the base substrate. In some embodiments, the MEA device is treated with a substance, including but not limited to poly(3,4-ethylenedioxythiophene) (PEDOT), to improve the sensitivity of the base electrode. In some embodiments, resin, including but not limited to Nafion resin, is applied to the MEA device, and a mold (e.g., a PDMS mold) is pressed into the resin to create in the resin, once cured, nanogrooves or nanopatterns as described herein. When used in culture, Nafion nanopatterns can promote uniaxial cellular alignment (see e.g., FIG. 4). In some embodiments, the substrate can be any substance that is ion permeable, highly durable at physiological temperatures, and/or inherently cell and protein adhesive.

In accordance with some embodiments, a biocompatible layer can be applied to the cell culturing surface of the MEA, e.g., as shown in FIG. 4. The biocompatible layer in accordance with some embodiments can be any biocompatible material, for example, a flexible polymer such as Polydimethylsiloxane (PDMS) or Polyurethane (PU). In accordance with some embodiments, the flexibility and/or hardness of the biocompatible layer can be selected to encourage a more physiological response of cells arranged upon it. For example, to the extent that muscle cells in vivo can normally be arranged upon an extracellular matrix substrate that is naturally flexible, and gives when the cells contract, it is contemplated that the use of a flexible biocompatible layer can permit cells arranged thereupon to behave in a manner that better approximates muscle tissue in vivo. In some embodiments, such an arrangement can contribute to more accurate prediction of, e.g., drug effects on muscle tissue in vivo using the multielectrode array constructs described herein. In accordance with some embodiments, the biocompatible layer can be a rigid, semi-rigid, or flexible material selected to approximate, e.g., bone, cartilage or an extracellular matrix to more closely approximate the hardness, flexibility and texture of the natural material on which the cells naturally develop.

In accordance with some embodiments, the MEA can be integrated with a flexible biocompatible layer (e.g., PDMS or PU), that possesses nanometer (or micrometer) scale features, such as grooves and/or ridges. In accordance with some embodiments, the nanometer grooves (or “nanogrooves”) can be in the range from 10 nm to 1000 nm in width, pitch, and/or depth, and can provide mechanical cues to the cells that are cultured on top of the nanogrooves. The nanogrooves and other nanometer scale features can be formed by well-known additive or subtractive fabrication processes, including etching (e.g., laser etching, chemical etching, dry etching and/or reactive ion etching) and stamping. The nanogrooves can be formed by capillary force lithography-based nanopatterning.

In some embodiments, the base substrate or the surface substrate of the MEA device can comprise at least one of polyglycolic acid (PGA), polylactic acid (PLA), poly(lactic-co-glycolic) acid (PLGA), polyanhydride, polycapralactone (PCL), polydioxanone, polyorthoester, PUA, GMA, or any combination thereof One of the most common polymers used as a biomaterial is the polyester copolymer poly(lactic acid-glycolic acid) (PLGA). PLGA is highly biocompatible, degrades into biocompatible monomers (e.g., if implanted) and has a wide range of mechanical properties making this copolymer and its homopolymers, PLA and PGA, useful as a cell culturing layer for cell deposition. The surface substrate layer can be porous or non-porous.

In some embodiments, other materials can be selected to be used as the surface substrate, i.e., the cell culturing layer material, which can be selected from the group consisting of hydroxyapatite (HAP), tricalcium phosphate (TCP), tetracalcium phosphate (TTCP), dicalcium phosphate anhydrous (DCPA), dicalcium phosphate dihydrate (DCPD), octacalcium phosphate (OCP), calcium pyrophosphate (CPP), collagen, gelatin, hyaluronic acid, chitin, and poly(ethylene glycol). In alternative embodiments, the cell culturing layer can also comprise additional material, for example, but are not limited to calcium alginate, agarose, types I, II, IV or other collagen isoform, fibrin, hyaluronate derivatives or other materials (see e.g., Perka C. et al. (2000) J. Biomed. Mater. Res. 49:305-311; Sechriest V F. et al. (2000) J. Biomed. Mater. Res. 49:534-541; Chu C Ret. al. (1995) J. Biomed. Mater. Res. 29:1147-1154; Hendrickson D A et al. (1994) Orthop. Res. 12:485-497). In some embodiments, a thermoresponsive polymer can be selected to be used as the surface substrate or as a component of the surface substrate. As a non-limiting example, poly(Nisopropylacrylamide) (PNIPAM) can be grafted to the surface of the nanofabricated substrate to facilitate cell sheet detachment, especially in embodiments comprising multi-layered cell sheets.

In some embodiments, the MEA device can comprise additional coatings. For example, but without wishing to be bound by theory, the cell-culturing surface of an MEA device can be modified with one or more bioactive agents, deposited or adsorbed on the polymer surface, to modify cell attachment, differentiation and maturation, adhesion-dependent cell signaling and/or electroconductivity of the device. Accordingly, in some embodiments, the MEA device can include within its polymer matrix, or on its surface, a bioactive agent that enhances maturation of the cultured cells, enhances survival of the cultured cells in response to toxic stimuli, enhances cell adherence to the polymer layer, and/or enhances action potential wave propagation across said the layer of cultured cells on the MEA device. In some embodiments, the MEA device may comprise a substrate base layer, and optionally, a surface layer.

In some embodiments, the MEA device for use in the methods and compositions as disclosed herein can additionally provide controlled release of bioactive agents to the cultured cells, for example, growth factors and other agents to sustain or control subsequent cell growth and proliferation of the cells coated on the MEA device of the technology described herein. In such a way, the cultured cells are supplied with a constant source of growth factors and other agents for the duration of the lifetime of the cell coated scaffold. In some embodiments, the growth factors and other agents are, neurotrophic factors, myotrophic factors, and/or cardiotrophic factors commonly known in the art.

As used herein, “neurotrophic factors” refers to substances that promote the growth, survival, and/or differentiation of developing neurons, mature neurons, and/or associated cells such as glial cells. Neurotrophic factors can include, but are not limited to neurotrophins, brain-derived neurotrophic factor (BDNF), nerve growth factor, neurotrophin-3, neurotrophin-4 (which is also known as NT4, NT5, NTF4, and NT-4/5), ciliary neurotrophic factor (CNTF) or other members of the CNTF family, glial cell line-derived neurotrophic factor (GDNF), artemin, neurturin, persephin, ephrins (e.g., ephrin A1, ephrin A2, ephrin A3, ephrin A4, ephrin A5, ephrin B1, ephrin B2, and ephrin B3). In some embodiments, the neurotrophic factor is a member of the Epidermal Growth Factor (EGF) family (e.g., a neuregulin (NRG) such as NRG1, NRG2, NRG3, NRG4) or a member of the Transforming Growth Factor (TGF) family (e.g., TGFα, TGFβ). In some embodiments, the neurotrophic factor is selected from the group consisting of glia maturation factor, insulin, insulin-like growth factor 1 (IGF-1), vascular endothelial growth factor (VEGF), fibroblast growth factor (FGF), platelet-derived growth factor (PDGF), pituitary adenylate cyclase-activating peptide (PACAP), interleukin-1 (IL-1), interleukin-2 (IL-2), interleukin-3 (IL-3), interleukin-5 (IL-5), interleukin-8 (IL-8), macrophage colony-stimulating factor (M-CSF), granulocyte-macrophage colony-stimulating factor (GM-CSF), and neurotactin. Examples of various neurotrophins are disclosed in Henderson (1996), Current Opinion in Neurobiology, 6 (1): 64-70; Binder et al. (2004) Growth Factors. 22 (3): 123-131; Johnson and Tuszynski (2008) Neurotrophic Factors, CNS Regeneration (Second Edition); U.S. Pat. No. 5,739,307; WO publication 1999/043813; U.S. Pat. No. 5,512,661; each of which is incorporated by herein in its entirety.

As used herein, “myotrophic factors” refers to substances that promote the growth, survival, and/or differentiation of myoblasts, developing myotubes, mature myotubes, and/or associated cells such as satellite cells. Myotrophic factors can include, but are not limited to hepatocyte growth factor (HGF), members of the fibroblast growth factor (FGF) family, members of the insulin-like growth factor (IGF) family (e.g., IGF1), platelet derived growth factor (PDGF), growth hormone (GH), dexamethasone, testosterone, anabolic steroids, CNTF, Leukemia inhibitory factor (LIF), ephrins. In some embodiments, the myotrophic factor can be a myogenic regulatory factor (MRF), i.e., a factor (e.g., a transcription factor) that regulates myogenesis, e.g., MyoD, Myf5, myogenin, and MRF4. In some embodiments, neurotrophic factors and/or myotrophic factors can include any factors that promote or rejuvenate the formation of neuromuscular junctions, including but not limited to agrin, growth differentiation factor 11 (GDF11; also known as bone morphogenetic protein 11 (BMP-11)), netrin, Wnt, TGF-β, and tumor necrosis factor-α (TNF-α). In some embodiments, a neurotrophic factor can also be a myotrophic factor. In some embodiments, a myotrophic factor can also be a neurotrophic factor. See e.g., US Patent Publication 2016/028766; Finkelstein et al., J Neurosci Res. 1996 Oct. 1, 46(1):122-8; Syverud et al., Cells Tissues Organs. 2016, 202(3-4): 169-179; Poon et al., Front Synaptic Neurosci. 2013, 5: 6; each of which is incorporated by reference herein in its entirety.

As used herein, “cardiotrophic factors” refers to substances that promote the growth, survival, and/or differentiation of developing cardiomyocytes and/or mature cardiomyocytes. Cardiotrophic factors can include, but are not limited to creatine, carnitine, taurine, cardiotrophic factors as disclosed in U.S. Patent Application Serial No. 2003/0022367 which is incorporated herein by reference in its entirety, TGF-beta ligands, such as activin A, activin B, insulin-like growth factors, bone morphogenic proteins, fibroblast growth factors, platelet-derived growth factor natriuretic factors, insulin, Leukemia Inhibitory Factor (LIF), Epidermal Growth Factor (EGF), TGF alpha, atrial natriuretic factor, cripto, and cardiac transcription regulation factors, such as GATA-4, Nkx2.5, and Mef2-C, and products of the BMP or cripto pathway. Other cardiac enhancing peptides include cellular differentiation agents, such as cytokines and growth factors, as disclosed herein. Examples of various cell differentiation agents are disclosed in U.S. Patent Application Serial No. 2003/0022367, or Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001; each of which is incorporated by reference herein in its entirety.

In some embodiments, the MEA device can include, on its surface or embedded in the cell culturing layer matrix, poly-L-lysine, poly-D-lysine, poly-orinithine, vitronectin or erythronectin. In some embodiments, the cell culturing layer can comprise an engineered polypeptide including CS1, RGD, domains in extracellular matrix proteins that bind to integrin receptors, and others well known to persons of ordinary skill in the art.

In some embodiments, the MEA device comprises, either coated on its surface or within its polymer matrix, one or more agents selected from the group consisting of sphingosine phosphate or an analog thereof, fluric acid, zFADvmk, cardiotropin, or a growth factor selected from the group consisting of FGF, HGF, IGF1, SDF1a, EGF, angiopoietin, BMP, erthyropoietin (EPO), GDNG, c-GSF, GDF9, HDNF, GDF, thrombopoietin, TGFα, TGFβ, TNFα, PIGF, PDGF, interleukins IL1-IL17 and VEGF. In some embodiments, one or more agents can be selected from the group consisting of an antibody, antigen, glycoprotein, lipoprotein, DNA, RNA, polysaccharide, lipid, growth hormone, organic compound, and inorganic compound.

Additionally, in some embodiments, the surface of the MEA device can be modified to include at least one of the agents selected from following group: (a) extracellular matrix proteins to direct cell adhesion and function (e.g., collagen, fibronectin, laminin, etc.); (b) growth factors to direct cell function specific to cell type (e.g., nerve growth factor, bone morphogenic proteins, vascular endothelial growth factor, etc.); (c) lipids, fatty acids and steroids (e.g., glycerides, non-glycerides, saturated and unsaturated fatty acids, cholesterol, corticosteroids, sex steroids, etc.); (d) sugars and other biologically active carbohydrates (e.g., monosaccharides, oligosaccharides, sucrose, glucose, glycogen, etc.); (e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins (e.g., selectins, immunoglobulins, hormones such as human chorionic gonadotropin, Alpha-fetoprotein and Erythropoietin (EPO), etc.); proteolipids (e.g., N-myristoylated, palmitoylated and prenylated proteins); and glycolipids (e.g., glycoglycerolipids, glycosphingolipids, glycophosphatidylinositols, etc.); (f) biologically derived homopolymers, such as polylactic and polyglycolic acids and poly-L-lysine; (g) nucleic acids (e.g., DNA, RNA, etc.); (h) hormones (e.g., anabolic steroids, sex hormones, insulin, angiotensin, etc.); (i) enzymes (types: oxidoreductases, transferases, hydrolases, lyases, isomerases, ligases; examples: trypsin, collegenases, matrix metalloproteinases, etc.); (j) pharmaceuticals (e.g., beta blockers, vasodilators, vasoconstrictors, pain relievers, gene therapy, viral vectors, anti-inflammatories, etc.); (k) cell surface ligands and receptors (e.g., integrins, selectins, cadherins, etc.); and (1) cytoskeletal filaments and/or motor proteins (e.g., intermediate filaments, microtubules, actin filaments, dynein, kinesin, myosin, etc.).

In some embodiments, the MEA device cell culturing surface can be coated with materials that promote electroconductivity. In some embodiments, such material can be selected from the group consisting of PEDOT, charcoal, graphene, graphene oxide, reduced graphene oxide, nanotubes, titanium (Ti) and gold (Au), whereby electrical conductivity, or other physico-chemical property of the cultured cells can be amplified from the whole monolayer of cells present on the cell culturing surface. In some embodiments, the MEA device comprises electroactive polymer fibers that yield fibers that exhibit crystalline structures in polar form due to strong electromagnetic fields. Exemplary systems and methods for aligning the fibers are disclosed in US Application 2009/0108503, which is incorporated herein in its entirety by reference.

Depending on the base substrate employed, examples of surface layers on the MEA device that are electroconductive materials, include for example, but are not limited to, a thin metallic or conducting surface deposit, e.g., graphene, graphene oxide and reduced graphene oxide (rGO), carbon nanotubes, tantalum, titanium, Ti—Al—V alloys, gold, chromium, metal oxides, semiconductor oxides, metal nitrides, semiconductor nitrides, polymers, biopolymers, or other alloys. In some embodiments, surface compositions can include, for example, graphene and graphene oxide, as well as tantalum, titanium, platinum or an oxide thereof In some embodiments, the conductive surface layer can include modified structures of the above identified materials, including for example, carbon nanotube structures, metallic (e.g., gold, silver, and titanium) projections (e.g., pillars, posts, bumps, and ridges), and nanocavities.

### B. Electrodes

Described herein are devices comprising a plurality of field potential electrodes and a plurality of impedance electrodes on the surface of a substrate. In some embodiments, the plurality of field potential electrodes is located on the surface of the first cell growth area and the plurality of impedance electrodes is located on the surface of second cell growth area. In some embodiments, the plurality of field potential electrodes and the plurality of impedance electrodes are connected to an electronic interface.

Any electrical interface to connect the plurality of field potential electrodes and impedance electrodes to the electronic systems can be used. Typically, the device can comprise leads that are covered by a film that connect the electrodes to an electronic circuit. In some embodiments, the device can be removably mounted in a socket that facilitates electrical connection between one or more electrodes and electronic components that can receive signals from the cultured cells or generate signals to be applied to the cultured cells. In some embodiments as demonstrated herein, an electrical signal received by an electrode can be transferred through an associated lead to a corresponding socket pin. The socket pin can be soldered or otherwise electrically connected to a circuit board trace or a wire that is connected to the electronic components or systems. The electronic components or systems can include a series of signal conditioning components (e.g., amplifiers and/or filters) and/or data acquisition (DAQ) components so that the signal can be digitally sampled and recorded. In some embodiments, the MEA device is fabricated in different sizes and is compatible with amplifier sockets, e.g., MCS 106 amplifier and the like.

In some embodiments, the data acquisition components can be connected to a computer comprising a software package for analysis of the data, e.g., LabVIEW (from National Instruments, Austin, Tex.), for visualization of the electronic signal. In some embodiments, the captured signal contains noise (e.g., low frequency or high frequency noise) which can be removed by known techniques (e.g., filtering, shielding or providing a well-grounded source) and/or software known to persons of ordinary skill in the art.

In some embodiments, the electrode array includes a substrate with metallization in a spectral pattern. In some embodiments, the electrode array can be formed by sputter coating a Cr/Au material directly on the substrate using a shadowing mask for the spectral pattern of the electrodes. In some embodiments, the electrode array can be formed in the center of a substrate with leads connecting to one or more external circuits connected through pads on the outside or edges of the substrate. The external circuits can include signal conditioning and/or filtering components as well signal multiplexing components, signal processing components, signal recording components, and/or signal generating components.

In some embodiments, the electrode can be used to modulate at least one biopotential of the cells cultured on the substrate, for example, but not limited to, a biopotential which is a functional parameter, including, but not limited to action potential duration (ADP), wave propagation, action potential frequency, beat frequency, action potential transmission, Vmax of the action potential, contraction force, peak to peak amplitude, end diastole to peak diastole rate and the like.

In accordance with some embodiments, the MEA devices can be formed in configurations compatible with standard integrated circuit devices such that the MEA devices can be removably inserted into integrated circuit type sockets in order to provide electrical connections between the MEA devices and external signal sensing and signal generating components. FIGS. 4A and 4B of US 2016/0017268 (which is incorporated herein by reference in its entirety) show an MEA device mounted in a wiring socket to facilitate external electrical connection. In accordance with some embodiments, various electrical interconnects can be used to connect the electrodes in the array with a peripheral pad. As shown in FIG. 4B of US 2016/0017268, Ag epoxy paint can be used to connect the leads to the pads without the use of heat which can damage a polymer cell culturing layer. Other metallization processes can also be used, for example, a second mask process that includes sputter deposition can be used to apply additional layers. Well known etching (e.g., chemical etching, laser etching, dry etching, reactive ion etching) techniques can be used to remove portions of the biocompatible cell culturing layer for electrical connections as well as to form the micrometer and nanometer grooves.

In accordance with some embodiments, the MEA device can be formed by a process that includes applying layers sequentially to build up cell culturing surface. The pads, leads, and optionally, electrodes can be applied to a base substrate using a first mask and sputter deposition of conducting material. A cell culturing layer of biocompatible polymer or dielectric material can be applied over the surface and the layer can include the micro/nano patterning formed on the surface or the micro/nano patterning can be formed in a subsequent step. Optionally, holes and conducting vias can be formed in the cell culturing layer to provide electrical connection to electrodes that can be applied to the micro/nano patterned surface. The optional surface electrodes can be applied to the surface using a second mask and a sputter deposition process. Other well-known thin and thick film metal deposition processes can be used to apply the leads and the pads to the biocompatible cell culturing layer and/or the base substrate material.

As shown in FIGS. 5A-5B and 6 of US 2016/0017268, the electrodes can be electrically connected to one or more signal generating devices and one or more signal processing devices. The signal generating devices can include electronic devices that produce electrical potentials intended to be sent to the cells and tissues cultured on the surface of the MEA to study the cell/tissue response. The signal processing devices include devices that receive signals from the cells and tissue and characterize the signals. The signal processing devices can include signal conditioning blocks such as amplifiers and filters, and data acquisition (DAQ) devices that digitally sample and characterize the signal. The signal processing devices can also include a storage device for storing the signals produced by the cells/tissue. In accordance with some embodiments, the signal processing device can include a personal computer (PC) having one or more processors and associated memories, equipped with an interface board to send and/or receive signals to more than one electrode simultaneously. The PC can include software, such as LabVIEW software, to visualize the signals.

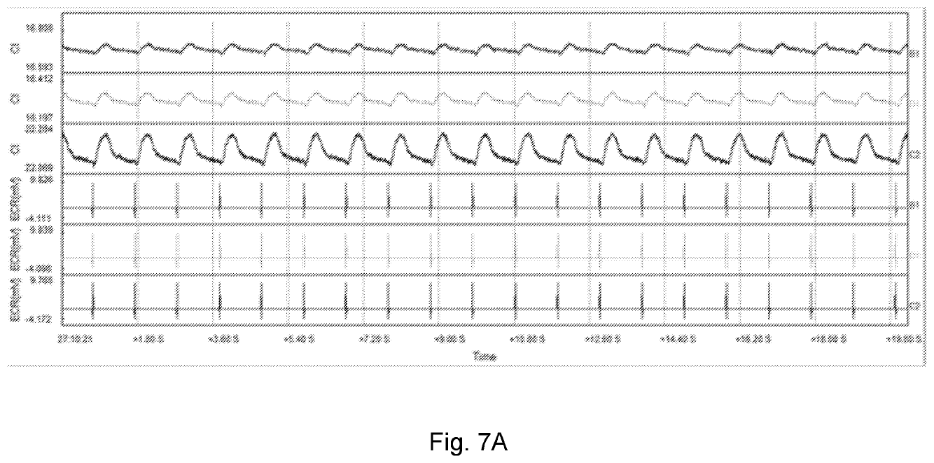

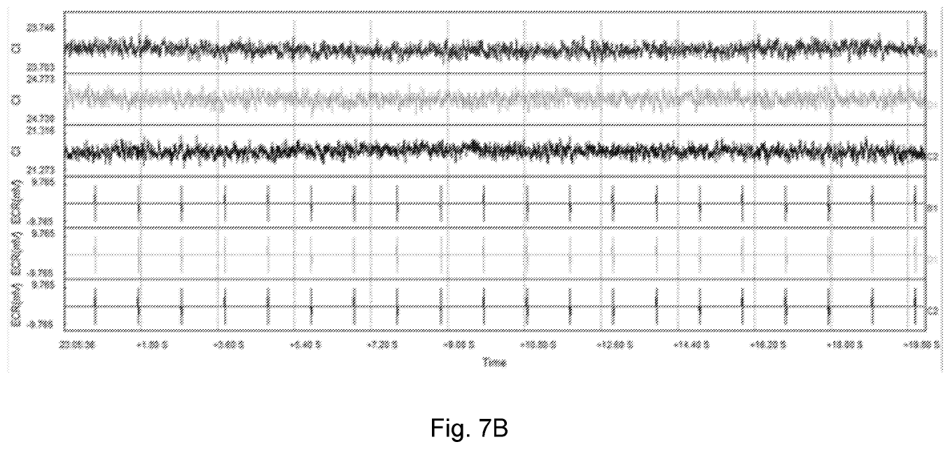

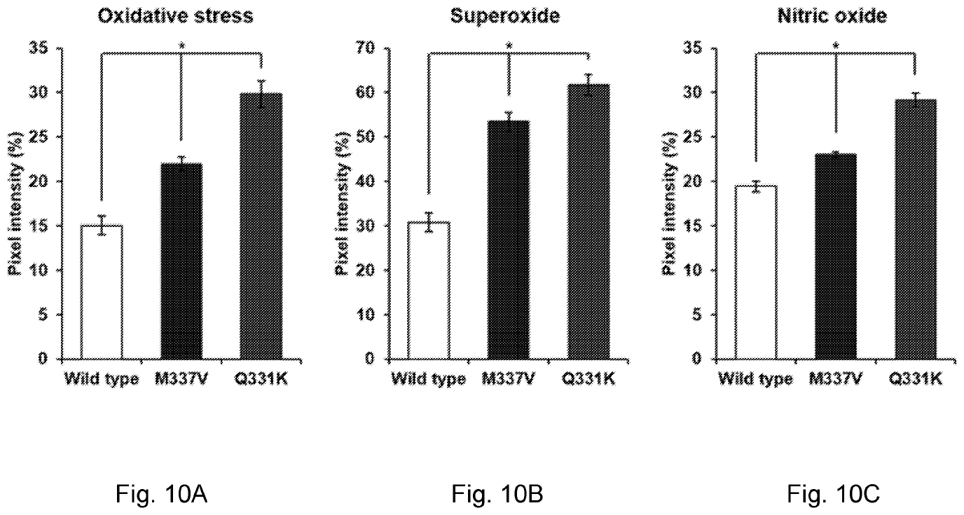

FIGS. 7, 8A-8B, 9A-9B, and 10 of US 2016/0017268 show images of the signals captured from various electrodes of the device shown in FIGS. 4A and 4B of US 2016/0017268. The signals are captured from pins 7, 8, 19 and 33 which connect to the adjacent electrodes of the MEA device. The signals were sampled at sampling rates varying from 100 Hz to 500 Hz and demonstrate that the location or orientation of the lead have little influence on the signal measurement.

In accordance with some embodiments, where the leads of electrodes are formed on the top of the cell culturing layer, it can be desirable to insulate the leads so they do not pick up signals from other cells that can interfere with signals received from cells in contact with the electrode. In accordance with some embodiments, the leads can be passivated by covering them with a thin layer of a biocompatible insulating material, such as parylene, shown in FIGS. 11B, 25B of US 2016/0017268. The insulating material can be on the order of 1.0-5.0 μm thick. In some embodiments, the insulating material can be SU-8.

In accordance with some embodiments, an MEA having a cell culturing surface can be modified as described herein. The MEA can include a plurality of electrodes arranged in any configuration on a cell culturing surface, the electrodes being connected by wires or leads to a pad located at or adjacent to a peripheral portion of the device.

(i) Field Potential Electrodes

Described herein is a device comprising a plurality of field potential electrodes and a plurality of impedance electrodes. In some embodiments, the plurality of field potential electrodes is located on the surface of the first cell growth area. In some embodiments, the plurality of field potential electrodes (FPE) is arranged in an array. In some embodiments, the field potential electrode is a microelectrode, i.e., microscopic.

In some embodiments, the device comprises a plurality of field potential electrodes on or adjacent to the cell culturing surface of the first cell growth area, e.g., at least about 2, or at least about 3, or at least about 4, or at least about 5, or at least about 6, or at least about 7, or at least about 8, or at least about 9, or at least about 10, or more than 10 field potential electrodes on or adjacent to the cell culturing surface. In some embodiments, the field potential electrodes can be of any geometry or shape for field potential electrodes known to persons of ordinary skill in the art. In some embodiments, the field potential electrodes can be arranged in a predefined pattern (e.g., circular, oval, elliptical, rectangular, square pattern) or an array. In some embodiments, the field potential electrodes can be arranged in a random or pseudo-random configuration. In some embodiments, some of the field potential electrodes can be arranged in a predefined pattern or array and some of the field potential electrodes can be randomly located within the predefined pattern or array, or outside of the predefined pattern or array.

In some embodiments, the field potential electrodes (FPE) are configured to receive an electrical signal via the electrical interface from a power source and configured to deliver an electrical stimulating signal to the surface of the first cell growth area.

In some embodiments, the field potential electrodes (FPE) are configured to monitor any one of: spontaneous, electrically-paced, or optically-paced activity of cells in contact with the field potential electrode. In some embodiments, said activity includes but is not limited to membrane potential, action potential, firing rate, and depolarizing spike amplitude.

In some embodiments, the field potential electrodes (FPE) are configured for one or both of: monitor any one of: spontaneous, electrically-paced, or optically-paced activity of cells in contact with the field potential electrode; and electrically stimulate cells present on the first cell growth area.

In some embodiments, the field potential electrode serves as an electro-stimulation electrode, thereby permitting electro-stimulation of excitable cells. That is, the field potential electrodes are configured to receive an electrical signal from a power source, thereby delivering an electro-stimulation signal to the cells on the first cell growth area and that contact the field potential electrodes.

In some embodiments, the plurality of field potential electrodes are not in an array, or interdigitated with any impedance electrodes.

In some embodiments, the field potential electrodes can electrically stimulate synaptic connections of cells present on the first cell growth area with the cells present on the second growth area.

In some embodiments, a plurality of field potential electrodes (FPE) can be provided in a variety of configurations. In addition, the plurality of FPE can be electrode structures having a same surface area.

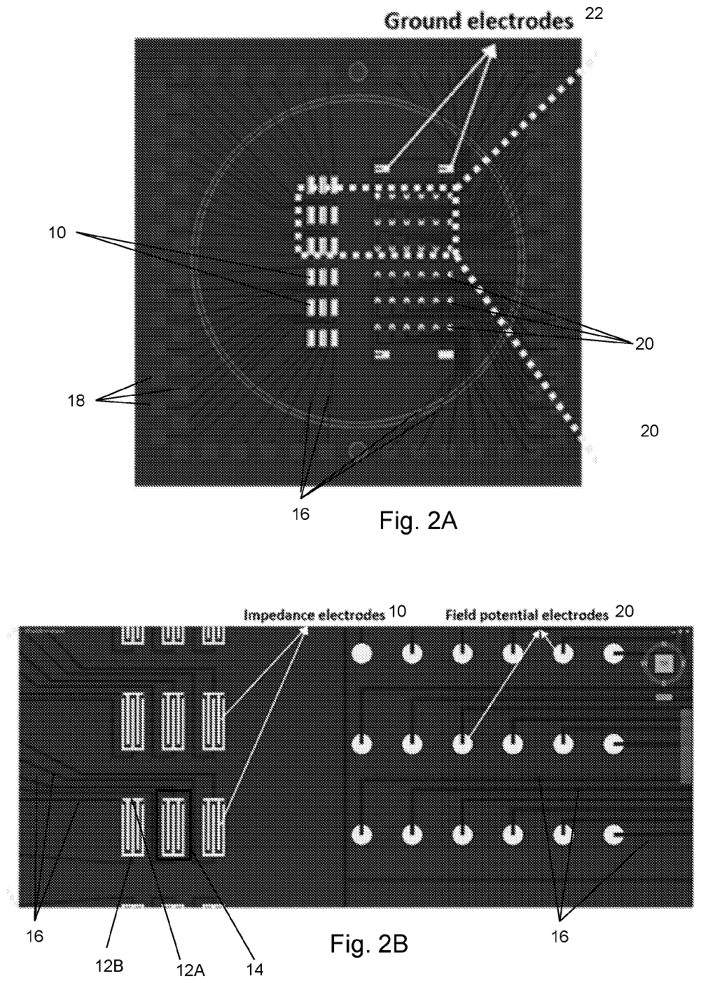

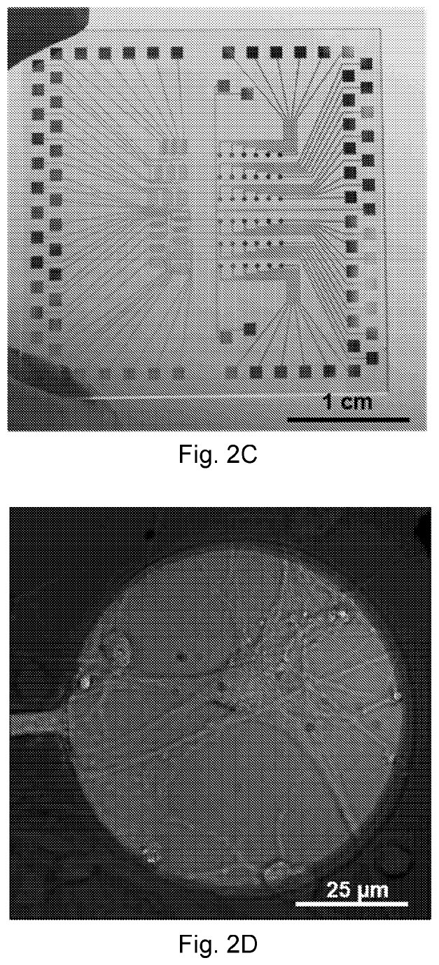

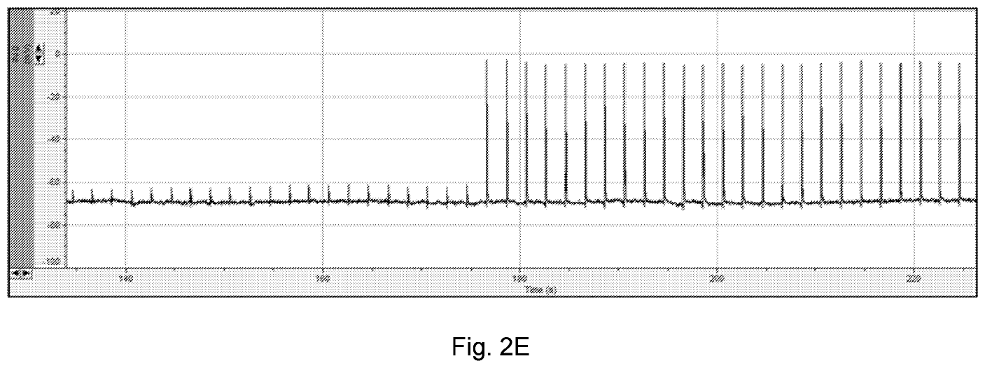

Examples of suitable configurations for the plurality of field potential electrodes are shown in FIGS. 2A-2C, which depict examples of the configuration of the plurality of field potential electrodes with a plurality of impedance electrodes. Turning to FIG. 2B as an exemplary configuration of the plurality of field potential electrodes, in some embodiments, one field potential electrode 20 can be configured as a circle. Each field potential electrode 20 can be connected to an electrical trace 16 that is covered by a non-conductive substrate, where the electrical trace 16 is connected to a connection pad 18.

In some embodiments, a field potential electrode 20 is a circle and has a diameter from about 10 μm to about 200 μm or from about 30 μm to about 100 μm. In some embodiments, a field potential electrode is 60μm and the distance between two field potential electrodes in an array is between about 1-5 mm, for example, about 1 mm, about 2 mm, or about 3 mm, or about 4 mm, or about 5 mm.

In some embodiments, the field potential electrodes may be provided in a variety of shapes and configurations so long as the configuration permits electro-stimulation. Electro-stimulation can be accomplished using a variety of different waveforms such as rectangular, ramp, sinusoidal signals and the like. Either uni-polarity or bi-polarity signals can be used, with signal amplitude ranging from −2.5 V to +2.5 V. In some embodiments, an electrical signal for stimulation of neurons for pacing myocytes is from between 1 V-2V, for example, an electrical signal between about 1.1V-1.3V. In some embodiments, the field potential electrodes can be configured to provide a voltage that is controlled at voltage resolutions up to 2 mV. Maximum frequency of the stimulation signals could be up to 50 kHz.

(ii) Impedance Electrodes

Described herein is a device comprising a plurality of field potential electrodes and a plurality of impedance electrodes (IE). In some embodiments, the plurality of impedance electrodes is located on the surface of the second cell growth area. In some embodiments, the plurality of impedance electrodes (IE) is arranged in an array. In some embodiments, the impedance electrode is a microelectrode, i.e., microscopic.

In some embodiments, a plurality of impedance electrodes is a plurality of impedance monitoring electrodes which are optionally communicatively coupled or connected to at least one analyzing module, for example an analyzing module in the form of the impedance analyzer, thereby permitting impedance monitoring of excitable cells on the second cell growth area that contact the impedance electrodes. In some embodiments, the plurality of impedance electrodes can be alternatively used for electrical stimulation of the excitable cells, e.g., muscle cells on the second cell growth area when not measuring impedance. In some embodiments, the plurality of impedance electrodes are not in an array, or interdigitated with any field potential electrodes.

In some embodiments, a plurality of impedance electrodes can be provided in a variety of configurations but is preferably a pair of interdigitated electrode structures, wherein each electrode structure comprises a plurality of electrode elements. In addition, the plurality of impedance electrodes can be electrode structures having a same surface area. In some embodiments the impedance analyzer monitors impedance at millisecond time resolution. In some embodiments, the plurality of impedance electrodes is an array of circle-in-line electrode elements

Examples of suitable configurations for the plurality of impedance electrodes are shown in FIGS. 2A-2C, which depict examples of the configuration of the plurality of impedance electrodes with the plurality of field potential electrodes. Turning to FIG. 2B as an exemplary configuration of the plurality of impedance electrodes, in some embodiments, one impedance electrode 12A can be configured in a branched formation and can be arranged to be interdigitated with another branched impedance electrode 12B. Such an impedance electrode pair 14 can be arranged as an array of a plurality of impedance electrodes 10. Each impedance electrode 12A, 12B of an impedance electrode pair 14 can be connected to an electrical trace 16 that is covered by a non-conductive substrate, where the electrical trace 16 is connected to a connection pad 18.

In some embodiments, the device comprises a plurality of impedance electrodes, for example, a plurality of impedance electrode pairs 14 on or adjacent to the cell culturing surface of the second cell growth area, e.g., at least about 2, or at least about 3, or at least about 4, or at least about 5, or at least about 6, or at least about 7, or at least about 8, or at least about 9, or at least about 10, or more than 10 impedance electrodes, or impedance electrode pairs 14, on or adjacent to the cell culturing surface. In some embodiments, the impedance electrodes can be of any geometry or shape for impedance electrodes known to persons of ordinary skill in the art. In some embodiments, the impedance electrodes can be arranged in a predefined pattern (e.g., circular, oval, elliptical, rectangular, square pattern) or an array. In some embodiments, an impedance electrode is configured in a branched formation 12A, 12B, and in some embodiments, two branched impedance electrodes can form a pair of impedance electrodes 14. In some embodiments, the impedance electrodes can be configured as a contiguous line in branched configuration (i.e.,). Alternatively, in some embodiments, the impedance electrodes are configured as electrode elements arranged as circles-in-line (i.e.,), and can be arranged in a branched configuration. In some embodiments, the impedance electrodes can be in a spiral configuration and a pair of interdigitated spirals of two impedance electrodes can form an impedance electrode pair.

In some embodiments, the device comprises at least one pair of impedance electrodes 14, each comprising two impedance electrode structures 12A, 12B, such as, in the form of a spiral configuration or an interdigitated configuration. In some embodiments, a pair of impedance electrodes 14 are fabricated on the second cell growth surface of the substrate, in which the pair comprises two impedance monitoring electrode structures 12A, 12B, each of which comprises multiple circle-on-line electrode elements, in which the electrode elements of one electrode structure 12A alternate with the electrode elements of the opposite electrode structure 12B. The pair of impedance monitoring electrodes 14 may be provided in configurations, such as interdigitated, circle-on-line, diamond-on-line, concentric, sinusoidal and castellated.

In some embodiments, where the impedance electrodes are configured as electrode elements as circles-in-line, the diameter of each circle is between 50-150 μm, and can be e.g., 90 μm, and in some embodiments, the center-to-center distance between two adjacent circle-in-line electrode elements is between 50-150 μm, for example, 110 μm.

In some embodiments, the plurality of impedance electrodes or impedance electrode pairs 14 can be arranged in a random or pseudo-random configuration. In some embodiments, some of the impedance electrodes can be arranged in a predefined pattern or array and some of the impedance electrodes can be randomly located within the predefined pattern or array, or outside of the predefined pattern or array.

Turning now to the pair of impedance electrodes 14 themselves, each pair includes two or more electrode structures that are constructed to have dimensions and spacing such that they can, when connected to an impedance analyzer, operate as a unit to generate an electrical field in the region of spaces around the impedance electrode structures. Preferably the electric field is substantially uniform across the pair of impedance electrodes. In preferred embodiments, the pair of impedance electrodes 14 includes two impedance electrode structures, each of which includes multiple electrode elements, or substructures, which branch from the electrode structure. In preferred embodiments, the electrode structures in each pair have substantially the same surface area.

Each of the two complementary impedance electrodes 12A, 12B in the impedance electrode pair 14 connect to a separate connection pad 18 that is preferably located at the edge of the substrate. In some embodiments, the array includes at least two pairs of impedance electrodes 14, or at least 3, 4, 5, 6, 7, 8, 9, 10, 11, 12, 14, 15, 16, 17, 18, 19, 20 impedance electrode pairs 14. In such embodiments it is preferred that each impedance electrode 12A, 12B of an impedance electrode pair 14 be assigned to a separate connecting pad, however the pairs could be electrically joined such as by connection at a shared connection pad or through an electronic switch.

Preferably, each impedance electrode pair 14 of the device has an approximately uniform electrode resistance distribution across the entire pair of electrodes. By “uniform resistance distribution across the pair” is meant that when a measurement voltage is applied across the electrode structures of impedance electrode pair 14, the electrode resistance at any given location of the pair is approximately equal to the electrode resistance at any other location on the pair. Preferably, the electrode resistance at a first location on the pair and the electrode resistance at a second location on the pair does not differ by more than 30%. More preferably, the electrode resistance at a first location on the impedance electrode pair 14 and the electrode resistance at a second location on the same pair does not differ by more than 15%. Even more preferably, the electrode resistance at a first location on the pair and a second location on the same pair does not differ by more than 5%. More preferably yet, the electrode resistance at a first location on the pair and a second location on the same pair does not differ by more than 2%.

Preferred arrangements for electrode elements that form an impedance electrode pair 14 and gaps between the electrodes and electrode buses in a given pair are used to allow all cells, no matter where they land and attach to the pair of impedance measurement electrodes to contribute similarly to the total impedance change measured for the pair. Thus, it is desirable to have similar electric field strengths at any two locations within any given pair of impedance measurement electrodes when a measurement voltage is applied to the pair. At any given location of the pair, the field strength is related to the potential difference between the nearest point on a first electrode structure of the pair and the nearest point on a second electrode structure of the pair. It is therefore desirable to have similar electric potential drops across the electrode elements and across the electrode buses of a given pair. Based on this requirement, it is preferred to have an approximately uniform electrode resistance distribution across the whole pair where the electrode resistance at a location of interest is equal to the sum of the electrode resistance between the nearest point on a first electrode structure (that is the point on the first electrode structure nearest the location of interest) and a first connection pad connected to the first electrode structure and the electrode resistance between the nearest point on a second electrode structure (that is the point on the first electrode structure nearest the location of interest) and a second connection pad connected to the second electrode structure.

Achieving an approximately uniform distribution across the impedance electrode pair 14 can be achieved, for example, by having electrode structures and electrode buses of particular spacing and dimensions (lengths, widths, thicknesses and geometrical shapes) such that the resistance at any single location on the pair is approximately equal to the resistance at any single other location on the pair. In most embodiments, the electrode elements (or electrode structures) of a given pair will have even spacing and be of similar thicknesses and widths, the electrode buses of a given pair will be of similar thicknesses and widths, and the electrode traces leading from a given pair to a connection pad will be of closely similar thicknesses and widths. Thus, in these preferred embodiments, an impedance electrode pair 14 is designed such that the lengths and geometrical shapes of electrode elements or structures, the lengths and geometrical shapes of electrode traces, and the lengths and geometrical shapes of buses allow for approximately uniform electrode resistance distribution across the impedance electrode pair 14.

In some embodiments of impedance measurement configurations, electrode structures comprise multiple electrode elements, and each electrode element connects directly to an electrode bus. Electrode elements of a first electrode structure connect to a first electrode bus, and electrode elements of a second electrode structure connect to a second electrode bus. In these embodiments, each of the two electrode buses connects to a separate connection pad via an electrical trace. Although the resistances of the traces contribute to the resistance at a location on the pair, for any two locations on the pair the trace connections from the first bus to a first connection pad and from the second bus to a second connection pad are identical. Thus, in some embodiments, trace resistances do not need to be taken into account in designing the geometry of the pair to provide for uniform resistances across the array.

It has been reported that electrode impedance may affect data quality in extracellular recordings. Accordingly, in some embodiments, the electrode impedance is chosen to maximize data quality. In some embodiments, at least one pair of impedance electrodes is a low impedance electrode. As used herein, “low impedance” refers to an electrode with an impedance of at most 0.1 mega-Ohms (MΩ). As a non-limiting example, at least one impedance electrode has an impedance of at most 0.05 MΩ, at most 0.1 MΩ. In some embodiments, at least one impedance electrode is a high impedance electrode. As used herein, “high impedance” refers to an electrode with an impedance of at least 1 MΩ. As a non-limiting example, at least one pair of impedance electrodes has an impedance of at least 1 MΩ, at least 2 MΩ, or at least 5 MΩ. See e.g., Neto et al., Front Neurosci. 2018; 12: 715, which is incorporated herein by reference in its entirety.

In some embodiments, the impedance electrodes (IE) are communicatively coupled via the connection pad 18 to an electrical interface to at least one analyzing module in the form of an impedance analyzer, thereby permitting impedance monitoring from excitable cells attached to the surface of the second growth area. In some embodiments, said monitoring can include contraction magnitude, contraction velocity, and relaxation speed.

(iii) Ground Electrodes and Electrical Traces

In some embodiments, the device comprising a plurality of field potential electrodes and a plurality of impedance electrodes (IE) also comprises a plurality of ground electrodes 22. Each ground electrode 22 is connected to an electrical trace 16 which is connected to a connection pad 18. In some embodiments, the plurality of ground electrodes are configured on the surface of the first cell growth area, and arranged near the array of the plurality the field potential electrodes 20. The ground electrodes can also be referred to reference electrodes. The ground electrode serves as the reference point in the electrical circuit from which voltages are measured. In essence, the ground electrode provides a baseline measurement from which fluctuations in voltage, detected by the recording electrodes (e.g., impedance electrode, field potential electrode), are compared in order to calculate amplitude and waveform. The ground electrode can be placed anywhere in the device as long as it is fluidically connected to the recording electrodes (e.g., impedance electrode, field potential electrode). The specific position of the ground electrode does not matter, per se, but its location may dictate changes in size of the ground electrode in order to effectively ground the system. In some embodiments, the device comprises at least 1, at least 2, at least 3, at least 4, or at least 5 ground electrodes.

In some embodiments, a ground electrode generally, can be a unitary or unbranched electrode and may be of a simple geometry such as a circle, a square and the like. In other embodiments, the ground electrode has a branched configuration, which may result in a large surface for the ground electrode. In some embodiments, the ratio of the surface area of the ground electrode to that of the impedance electrodes and field potential electrodes is more than 2. In other embodiments, the ratio of the surface area of the ground electrode to that of the impedance electrodes and field potential electrodes is 10 or more than 10. In still other embodiments, the ratio of the surface area of ground electrode to that of the impedance electrodes and field potential electrodes is 100 or more than 100. In other embodiments the ratio of the surface area of the ground electrode to that of the impedance electrodes and field potential electrodes is 1000 or more than 1000. In other embodiments the ratio of the surface area of the ground electrode to that of the impedance electrodes and field potential electrodes is 10,000 or more than 10,000.

Preferably, each of the field potential electrodes and impedance electrodes of an electrode array is connected to a separate connection pad 18, which is preferably located at the edge of the substrate. Connecting the field potential electrodes 20 or the impedance electrodes 12A, 12B to the connection pads 18 can be performed by applying electrical traces 16 of conductive material therebetween. This facilitates connection to a suitable power source by providing an interface at which the power source can connect. Connection to the connection pads is generally performed through the use of electrically conductive pins, clips or the like.

In some embodiments, the device can further comprise an extracellular recording electrode, for example, for extracellular recording that is conducted by amplifying and recording electrical voltage signals between one or more of a field potential electrode 20, an impedance electrode 10, or impedance electrode pair 14, or a ground electrode 22. Such electrical voltages are induced on the electrodes as a result of ionic current or movement through cell culture media or solution supporting the cells during the experiment as a result of opening and/or closing of different ion channels across cell membrane during the action potential duration. In order to achieve improved consistency and reproducibility of the recorded voltage signals, it is desirable to minimize the contribution of any electrical signal from the ground electrode 22 to the recorded voltage signals and to ensure that the majority, if not all, of the recording voltage signals are derived from that on the impedance electrodes 10. Thus, generally, it is desirable and it is recognized for the ground electrodes to have small electrode impedances. The small electrode impedance is achieved by using ground electrodes with large effective surface areas by increasing the ratio of the surface area of the ground electrodes to that of recording electrode by a factor of a hundred, even thousands of times. For example, FIG. 2A shows a schematic representation of such field potential electrodes 20 and impedance electrode pairs 10 spatially separated on a non-conductive substrate, including a plurality of ground electrodes 22. As shown throughout FIG. 2A, preferably the ground electrode 22 is positioned towards the perimeter of the plurality of field potential electrode array 20; however, it does not physically contact any of the field potential electrodes 20.

### II. Cell Types

As described herein, cells are plated onto, attach to, differentiate, and/or function on the surface substrate of the MEA device. In some embodiments, these cells are electroconductive cell types. As used herein, “electroconductive cell” refers to a cell being able to conduct, generate, and/or respond to an electrical signal. It should be noted that at some level, any cell can conduct electricity; however, relevant electroconductive cells have the normal physiological function or design to generate, propagate, or respond to an electrical stimulus. In some embodiments, the electroconductive cells include muscle cells (including, but not limited to, cardiomyocytes, skeletal muscle myocytes and smooth muscle myocytes) and neuronal cells. In some embodiments, the electroconductive cells are human cells. In some embodiments, the electroconductive cells are derived from stem cells, e.g., ES cells and/or induced pluripotent stem cells (iPSC). In some embodiments, a cell culture system described herein can comprise a plurality of types of electroconductive cells.

In some embodiments, the device comprises a plurality of neuronal cells on a first cell growth area and a plurality of contractile cells or muscle cells on a second cell growth area. In some embodiments, a plurality of field potential electrodes is located on the surface of the first cell growth area (i.e., in association with the plurality of neuronal cells) and the plurality of impedance electrodes is located on the surface of second cell growth area (i.e., in association with the plurality of contractile cells or muscle cells).

In some embodiments, the cell types are paired within the device to be physiologically relevant, i.e., the first growth area and second growth area comprise a pair of muscle and neuron cell types that naturally occur together, i.e., the neuronal cell type naturally innervates the muscle cell type. For example, skeletal muscle innervation by motor neurons, cardiac muscle innervation by sympathetic neurons, or smooth muscle innervation by the autonomic nervous system. In some embodiments, the device comprises a non-naturally occurring pairing of a muscle cell type and a neuronal cell type. Additional examples of electroconductive cells can include osteocytes, monocytes, and macrophages; see e.g., WO 2018/134366, which is incorporated herein by reference in its entirety.

Accordingly, in some embodiments, the plurality of neuronal cells comprise motor neurons, and the plurality of muscle cells comprise skeletal muscle cells. In some embodiments, the plurality of neuronal cells comprise sympathetic neurons, and the plurality of muscle cells comprise smooth muscle cells. In some embodiments, the plurality of neuronal cells comprise sympathetic neurons, and the plurality of muscle cells comprise cardiac muscle cells or cardiomyocytes.

Electroconductive cells can include both naturally-occurring electroconductive cells (e.g., a muscle cell or neuron) or cells that have been engineered, e.g., genetically modified or transfected to exhibit electroconductive activity. By way of non- limiting example, a cell engineered to express at least one voltage-gated ion channel can be an engineered electroconductive cell. One of skill in the art is familiar with methods for engineering cells, which can include, but are not limited to, genetic modification, homologous recombination, transient expression, and protein transfection and can be accomplished with one or more various vectors, e.g., plasmids, naked DNA, or viral vectors.

In some embodiments, described herein is a culture cell comprising the electroconductive cells disclosed herein. In some embodiments, the cell culture can further comprise non-electroconductive cells. Non-limiting examples of non-electroconductive cells include neuronal glial cells, as described further herein, and muscle satellite cells.

In some embodiments, the cell culture system can comprise a plurality of electroconductive cells. In some embodiments, the plurality of cells can form a monolayer of cells. In some embodiments, the plurality of cells can form a multiple layers of cells. As a non-limiting example, the plurality of cells can form 1 layer, 2 layers, 3 layers, 4 layers, 5 layers, or at least 6 layers of cells. In some embodiments, the plurality of cells can form a tissue, e.g., a muscle tissue or nerve tissue. In some embodiments, the cell culture system can further comprise non-electroconductive cells, e.g., fat cells or epithelial cells.

### A. Neurons

Described herein is a device comprising electroconductive cells. In some embodiments, the electroconductive cells can comprise a plurality of neuronal cells. In some embodiments, the neuronal cells can be from the brain, the spinal cord, dorsal root sensory ganglia, or autonomic ganglia. In some embodiments, the neuronal cells can be motor neurons, sensory neurons, interneurons, autonomic neurons, sympathetic neurons, or parasympathetic neurons. In some embodiments, the neuronal cells can be unipolar, bipolar, multipolar, or pseudounipolar neurons. In some embodiments, the neuronal cells can be myelinated or non-myelinated.

In some embodiments the neuronal cells can comprise cells from a neuronal cell line or a neuron-like cell line including but not limited to neuro-2a cells, PC12 cells, SH-SY5Y cells, F11 cells, or NSC-34 cells. In some embodiments, the neuronal cells can be derived as a primary cell line, e.g., isolated from a patient or animal model nervous tissue sample or nervous tissue biopsy (e.g., from the brain, the spinal cord, dorsal root sensory ganglia, or autonomic ganglia).

In some embodiments, the plurality of neuronal cells are derived from iPSCs obtained from a healthy subject. In some embodiments, the plurality of neuronal cells are derived from iPSCs from a subject with a neurodegenerative disease or disorder or a myopathy.

As described herein, human induced pluripotent stem cell (hiPSC)-derived motor neurons have been generated from patients with multiple peripheral neuropathic conditions, including amyotrophic lateral sclerosis (e.g., ALS). However, the ability to effectively model such conditions in vitro using these cells has yet to be achieved due to the complexity of generating robust and functionally competent neuromuscular junctions (NMJs) in culture. In addition, the wide array of mutations thought to cause the disease reduces the value of single mutant lines as representative models of ALS. In some embodiments, a high throughput platform, as described herein, promotes NMJ development across a multiplexed assay housing multiple patient lines, which substantially and positively impacts advanced therapy development, drug efficacy/toxicity screening, and mechanistic studies of neuronal and NMJ pathophysiology in peripheral neuropathic conditions such as ALS.

(i) Neuromuscular Junctions

Described herein are devices, methods, assays and systems comprising an in vitro model of neuromuscular junction (NMJ) development and function. As used herein, “neuromuscular junction” refers to the synapse formed between a motor neuron and a contractile cell (e.g., a skeletal muscle cell). The NMJ comprises a presynaptic region of a neuron, a synapse (i.e., synaptic cleft), and a post-synaptic region of a contractile cell. At the NMJ, the electrical signal of an action potential from the neuron can be translated into the chemical signal of a neurotransmitter, which binds to neurotransmitter receptors on or in the contractile cell. Examples of neurotransmitters are well known in the art and include but are not limited to: glutamate, aspartate, D-serine, γ-aminobutyric acid (GABA), glycine, nitric oxide (NO), carbon monoxide (CO), hydrogen sulfide (H2S), dopamine (DA), norepinephrine (noradrenaline; NE, NA), epinephrine (adrenaline), histamine, or serotonin (SER, 5-HT).

In a preferred embodiment, NMJs are located in the second cell growth area between the plurality of muscle cells and cell extensions (e.g., axons, dendrites) from the plurality of neuronal cells. In some embodiments, NMJs are located in first cell growth area, axon outgrowth area, the second cell growth area, and/or additional cell growth areas.

In some embodiments, NMJ development (e.g., of cells grown in a device as described herein) is compared to a healthy control. In some embodiments, determining NMJ development can comprise measuring at least one of the following: the average diameter of neurotransmitter receptor “plaques” on the post-synaptic muscle cell; the degree of post-synaptic membrane invagination; the number of co-localized pre-synaptic and post-synaptic structures; or the number of poly-innervated vs. singularly innervated vs. denervated fibers (e.g., using stains for post-synaptic markers such as neurotransmitter receptors and stains for pre-synaptic markers such as synaptic vesicle proteins) (see e.g., Table 3). In some embodiments, the plurality of electroconductive cells is classified as having abnormal NMJ development if a measurement of at least one of the above is statistically different than that of a healthy control.

In some embodiments, NMJ functionality (e.g., of cells grown in a device as described herein) is compared to a healthy control. In some embodiments, determining NMJ functionality comprises activating the plurality of neuronal cells in the first cell growth area (e.g., applying an electrical stimulus using the field potential electrodes and/or a chemical stimulus including but not limited to glutamate) and measuring changes in the contractile activity (e.g., frequency or amplitude of contraction) of the plurality of muscle cells in the second growth area (e.g., through measurements of the impedance electrodes). In some embodiments, the plurality of electroconductive cells is classified as having abnormal NMJ functionality if a measurement of at least one of the above is statistically different than that of a healthy control.

In some embodiments, synaptic agonists or synaptic antagonists (see e.g., Table 2) can be added to at least one chamber or cell growth area of the device to modulate NMJ functionality. In some embodiments, measurement of NMJ functionality can be used as a measure of NMJ development. In some embodiments, synchronous contraction of the plurality of muscle cells can be used as a measurement of NMJ development and/or NMJ functionality.

(ii) Optogenetic Neurons

In some embodiments, the plurality of neuronal cells can comprise optogenetic neuronal cells. In some embodiments, light pulses can be employed to depolarize an engineered neuron. In some embodiments, neuronal cells are engineered to express the channelrhodopsin-2 gene (ChR2) and/or the Volvox carteri light-activated ion channel protein (VChR1), and optionally a reporter (e.g., a fluorophore), e.g., by using a viral vector or any other method of genetic engineered as known in the art. In some embodiments, neuronal cells are engineered to expressed ChR2 using an adeno-associated virus (AAV) vector (e.g., rAAV1-hSyn-ChR2(H134R)-mCherry). In some embodiments, viral particles are generated via a transfection protocol as known in the art. See e.g., Jazayeri et al. Nature neuroscience. 2012; 15(10):1368-70; Cao et al. Gene therapy. 2002; 9(18):1199-20646; U.S. Pat. Nos. 8,716,447; 8,815,582; 10,220,092; each of which is incorporated by reference herein in its entirety.

### B. Muscle Cells

Described herein is a device comprising electroconductive cells. In some embodiments, the electroconductive cells can comprise a plurality of contractile cells. In some embodiments, the electroconductive cells can be a plurality of muscle cells, also referred to herein as myocytes. In some embodiments, the muscle cells can be skeletal muscle cells (also called myofibers or skeletal muscle fibers or skeletal myocytes). In some embodiments, the muscle cells can be cardiomyocytes. In some embodiments, the muscle cells can be cardiac pacemaker cells. In some embodiments, the muscle cells can be smooth muscle cells.

In some embodiments, the plurality of muscle cells is derived from iPSCs obtained from a healthy subject or a subject with a neurodegenerative disease or disorder or a myopathy. In some embodiments, the plurality of muscle cells on the second cell growth area is a 2D monolayer (i.e., one layer of muscle cells). In some embodiments, the plurality of muscle cells on the second cell growth area is an engineered 3D skeletal muscle construct (see e.g., Example 4; see e.g., Jiao et al. ACS Nano. 2014, 8(5):4430-9).

In some embodiments the muscle cells can comprise cells from a muscle cell line or a muscle-like cell line or a muscle progenitor cell line including but not limited to C2C12 cells, RD (ATCC® CCL-136), A-673 (ATCC® CRL-1598), SJCRH30 [RC13, RMS 13, SJRH30] (ATCC® CRL-2061), L6 (ATCC® CRL-1458), NOR-10 (ATCC® CCL-197), So18 (ATCC® CRL-2174), Hs 729 [Hs 729T] (ATCC® HTB-153), and the like. In some embodiments, the muscle cells can be derived as a primary cell line, e.g., myoblasts and/or myofibers isolated from a patient or animal model muscle sample or muscle biopsy (e.g., Primary Human Skeletal Muscle Cells). In some embodiments, the muscle sample or muscle biopsy can be from a skeletal muscle, a smooth muscle (e.g., the wall of a visceral organ), or heart tissue.

### C. Additional Cell Types

Described herein is a device comprising electroconductive cells. In some embodiments, the device can further comprise at least one additional cell type on any one or more of: the first cell growth area, the second cell growth area, the axon outgrowth area, and/or an additional cell growth area. In some embodiments, the additional cell type can comprise non-electroconductive cells. In some embodiments, the additional cell type is selected from any of: Schwann cells, microglia, astrocytes or satellite cells.

In some embodiments, the additional cell type is Schwann cells, wherein the Schwann cells are located on the first cell growth area or axon outgrowth area, or both. In some embodiments, the Schwann cells are located in an additional cell growth area and/or the axon outgrowth area. In some embodiments, the Schwann cells are associated with the cell projections (e.g., axon) of the neuronal cells, e.g., as a myelin sheath. In some embodiments, the device can be used to study NMJ development and/or NMJ functionality in the context of myelinated neurons (e.g., presence of Schwann cells) vs. unmyelinated neurons (absence of Schwann cells).

In some embodiments the Schwann cells can comprise cells from a Schwann cell line including but not limited to sNF02.2 (ATCC® CRL-2885), sNF94.3 (ATCC® CRL-2886), S16 (ATCC® CRL-2941), S16Y (ATCC® CRL-2943), hTERT NF1 ipNF95.11b (ATCC® CRL-3390), S42 (ATCC® CRL-2942), SW10 (ATCC® CRL-2766), RT4-D6P2T (ATCC® CRL-2768), RSC96 (ATCC® CRL-2765), R3 [33-10ras3] (ATCC® CRL-2764), and the like. In some embodiments, the Schwann cells can be derived from a primary Schwann cell line, e.g., from a patient or animal model sample or biopsy. In some embodiments, the Schwann cells can be derived from iPSCs obtained from a healthy subject or a subject with a neurodegenerative disease or disorder or a myopathy.

In some embodiments, the additional cell type is microglia (i.e., microglial cells), wherein the microglia are located on the first cell growth area or axon outgrowth area, or both. In some embodiments, the microglia are located in an additional cell growth area and/or the axon outgrowth area.

In some embodiments, the additional cell type is astrocytes, wherein the astrocytes are located on the first cell growth area or axon outgrowth area, or both. In some embodiments, the astrocytes are located in an additional cell growth area and/or the axon outgrowth area.

In some embodiments, the additional cell type is satellite cells, wherein the satellite cells are located on the second cell growth area or an additional growth area, or both.

In some embodiments, the additional cell type can be any cell type known to associate with neuronal cells, e.g., glial cells, oligodendrocytes, ependymal cells, fibroblasts, etc. In some embodiments, the additional cell type can be any cell type known to associate with muscle cells, e.g., fibroblasts, endothelial cells, etc.

In some embodiments, the additional cell type is located in the first cell growth area, the axon outgrowth area, the second cell growth area, an additional cell growth area, or any combination thereof. In some embodiments, the additional cell type is derived from a cell line, a primary sample, or an iPSC.

In some embodiments, the device comprises at least 1, at least 2, at least 3, at least 4, at least 5, at least 6, at least 7, at least 8, at least 9, or at least 10 different additional cell types as described herein.

D. iPSC-Derived Cells

In one aspect, described herein is a cell culture system comprising a multi-electrode array as described herein and a progenitor of an electroconductive cell present on the cell culture surface. In some embodiments, the progenitor cell can be a cardiac progenitor cell, a myogenically committed cell, a neuron progenitor cell, a stem cell, an embryonic stem cell, an iPS cell, an adult stem cell. In some embodiments, the iPSC can be a human iPSC (hiPSC), such as the WTC11 cell line or the UC-2 cell line. In some embodiments, the cell culture system can further comprise electroconductive cells and/or non-electroconductive cells.

In some embodiments, the cells comprised by a cell culture system described herein, e.g., electroconductive cells can comprise cells obtained from (e.g., a primary cell line) or descended from cells obtained from a subject with a muscle, cardiac, or neuronal disease. In some embodiments, the muscle, cardiac, or neuronal disease can be a disease characterized or caused by aberrant electroconductive activity. In some embodiments, the muscle, cardiac, or neuronal disease can be a disease characterized or caused by inadequate neuromuscular junction formation or function. Non-limiting examples of such muscle, cardiac, and neuronal diseases can include arrhythmia, long QT syndrome, heart block, atrial fibrillation, bradycardia, tachycardia, ventricular fibrillation, Adams-Stokes disease, atrial flutter, Wolff-Parkinson White syndrome, peripheral neuropathies, Charcot-Marie-Tooth disease, and the like. In some embodiments, the cells can comprise a genetic mutation known to be associated with or to cause a muscle, cardiac, or neuronal disease. In some embodiments, the cells can comprise a genetic background known to be associated with or to cause a muscle, cardiac, or neuronal disease. In some embodiments, the cells can be obtained from, or descended from cells obtained from as ethnic background known to be associated with a muscle, cardiac, or neuronal disease.

In some embodiments, the electroconductive cells can be electroconductive cells modified to comprise a mutation associated with a neurodegenerative disease or disorder or a myopathy, e.g., engineered to comprise such a mutation. By way of non-limiting example, ALS is associated with certain mutations in, e.g., TDP-43, C9orf72, SOD1, TARDBP, ALS2, SETX, SPG11, FUS, VAPB, ANG, FIG4, OPTN, ATXN2, VCP, UBQLN2, SIGMAR1, CHMP2B, PFN1, ERBB4, HNRNPA1, MATR3, TUBA4A, ANXA11, NEK1, KIF5A, CHCHD10, SQSTM1, TBK1, HNRNPA2B1, C21orf2, CCNF, and TIA1. One of skill in the art is aware of further examples.

In some embodiments, the electroconductive cells can be electroconductive cells modified to comprise a mutation associated with a neuronal, muscle, or cardiac disease, e.g., engineered to comprise such a mutation. By way of non-limiting example, Charcot-Marie-Tooth disease is associated with certain mutations in, e.g., PMP22, MPZ, LITAF, EGR2, NEFL, KIF1B, MFN2, RAB7A, LMNA, MED25, TRPV4, GARS, HSPB1, GDAP1, DYNC1H1, LRSAM1, MTMR2, and SBF2, among others and long QT syndrome has been linked to mutations in KCNQ1, KCNH2, hERG, MiRP1, SCN5A, ankyrin B, KCNE2, KCNJ2, CACNA1C, Caveolin 3, SCN4B, AKAP9, SNTA1, and GIRK4. One of skill in the art is aware of further examples.

In some embodiments, mutations can be engineered into cells using any known method in the art, including but not limited to CRISPR-Cas, TALEN, zinc finger nuclease (ZFN), viral systems such as rAAV, and transposons.

In some embodiments, the plurality of neuronal cells and the plurality of muscle cells are both derived from healthy subject(s). In some embodiments, the plurality of neuronal cells and the plurality of muscle cells are both derived from patient(s) with a neurodegenerative disease or disorder or a myopathy or engineered to comprise mutation(s) associated with a neurodegenerative disease or disorder or a myopathy. In some embodiments, the neurodegenerative disease or disorder or a myopathy or mutation(s) associated with a neurodegenerative disease or disorder or a myopathy can be the same or different in the plurality of neuronal cells and the plurality of muscle cells. In some embodiments, the plurality of neuronal cells is derived from healthy subject(s), and the plurality of muscle cells is derived from patient(s) with a neurodegenerative disease or disorder or a myopathy or engineered to comprise mutation(s) associated with a neurodegenerative disease or disorder or a myopathy. In some embodiments, the plurality of muscle cells is derived from healthy subject(s), and the plurality of neuronal cells is derived from patient(s) with a neurodegenerative disease or disorder or a myopathy or engineered to comprise mutation(s) associated with a neurodegenerative disease or disorder or a myopathy.

### III. Array

In one aspect described herein is a device for monitoring the electrical communication between two different electrically excitable cell types, comprising at least one module on a substrate. In some embodiments, the device comprises an array of modules on the substrate. In some embodiments, the MEA device is configured as an array.

In some embodiments, the device comprises a plurality of modules, for example at least 2 modules, at least 3 modules, at least 4 modules, at least 5 modules, at least 6 modules, at least 7 modules, at least 8 modules, at least 9 modules, at least 10 modules, at least 15 modules, at least 20 modules, at least 25 modules, at least 30 modules, at least 35 modules, at least 40 modules, at least 45 modules, at least 50 modules, at least 55 modules, at least 60 modules, at least 65 modules, at least 70 modules, at least 75 modules, at least 80 modules, at least 85 modules, at least 90 modules, at least 95 modules, or at least 100 modules on the substrate.

In some embodiments, the modules can be arranged in a predefined pattern (e.g., circular, oval, elliptical, rectangular, square pattern) or an array. In some embodiments, the modules can be arranged in a grid-like array, e.g., a 2×2, 2×3, 4×6, 6×8, 8×12, 16×24, 32×48 etc. array. In some embodiments, the modules can be arranged in a random or pseudo-random configuration. In some embodiments, some of the modules can be arranged in a predefined pattern or array and some of the modules can be randomly located within the predefined pattern or array, or outside of the predefined pattern or array.

In some embodiments, the modules can be physically adjacent, e.g., share at least one chamber wall. In some embodiments, the modules can share electrical inputs and/or electrical outputs. In some embodiments, some or all of the modules can be connected to same electrical interface. In some embodiments, some or all of the modules can be connected to different electrical interfaces.

In some embodiments, the modules can be physically separated, e.g., in separate wells of a multi-well plate. In some embodiments, the modules can be configured to be used in a 6-well plate, 12-well plate, 24-well plate, 96-well plate, a 384 well plate, or a 1536 well plate. In some embodiments, at least two modules can be in the same well of a multi-well plate.

### IV. Uses and Assays

Although primitive synaptic contacts have been demonstrated within in vitro systems previously, the MEA device as disclosed herein can be used to assess the electrical communication between two spatially separated cell types, including for assessment of the neuromuscular junction.

### A. Uses

In one aspect, described herein is a method for measuring the electrical conductance from one cell type to a second cell type comprising: providing a device as described herein, wherein the device comprises a first cell type on the first cell growth area, and a second cell type on the second cell growth area, and wherein the first cell type extends axons across the axon outgrowth area to the second cell type in the second cell growth area; and providing electrical stimulation to the first cell type via the field potential electrodes, and recording electrical activity of the second cell type via the impedance electrodes.

In some embodiments, the first cell type is a plurality of neuronal cells. In some embodiments, the second cell type is a plurality of contractile or a plurality of muscle cells. In some embodiments, the plurality of muscle cells is a monolayer or an engineered skeletal muscle construct.

A central advantage to systems described herein is that the device can be used to monitor the effect on the electroconductivity of one cell population on another cell population. That is, as the field potential electrodes 20 and the impedance electrodes 20 are spatially separated, one can separate and monitor the effect of the electroconductive signals of the neuronal cells that are stimulated by the field potential electrodes, and monitor the effect on the muscle cells using the impedance electrodes. Therefore, the device permits the ability to separate, and distinctly analyze the electrical stimulation of one cell population and the effect on the cell population by recording from the impedance electrodes using one single instrument, namely, the electrode array device. This is accomplished by using field potential electrodes of neuronal cells and impedance monitoring of spatially separated muscle cells, by at least one pair of impedance electrodes respectively.

The electrical stimulation of neuronal cells in one location on the device, and impedance-based monitoring of muscle cells at a different location on the device, fills a major technological gap in monitoring the interaction of neurons and muscle cells in vitro. Moreover, as the device is configured with a nanotextured surface for optimal neuronal maturation, and optimal muscle cell, e.g., skeletal muscle or cardiomyocyte maturation, the device provides a valuable research and drug development tool to assess diseases and therapeutic agents for the treatment of a variety of neuromuscular diseases, myopathies and neurodegenerative diseases. Also, the device and systems as disclosed herein can also record action potentials of muscle cells trigged by neurons e.g. skeletal muscle action potentials or cardiomyocyte action potentials or field potentials, as well as activity of the NMJ, allowing detailed electrophysiological analysis and assessment of the neuron-muscle cell interaction, for relatively short durations at a time. Furthermore, the ability of electrostimulating neuronal cells, which can activate muscle cells via the NMJ allow for electrophysiological measurement and impedance measurement of otherwise isolated muscle cells. The synchronization of electrostimulation of neurons with impedance measurement of the spatially distinct muscle cells is valuable for predicting the effect of neuronal-muscle interaction, particularly where either the neuronal cells, or the muscle cells, or both, are derived from a diseased subject.

As an exemplary example, one can assess the effect of electrostimulation of neuronal cells derived from a subject with a neurodegenerative disease to see the effect on the pace of myocytes, as well to monitor the morphological or differentiative behavior of myocytes that are electrically connected to neurons from diseases subjects in vitro. Similarly, one can assess the effect of electrostimulation of neuronal cells derived from a subject a healthy subject to see the effect on the pace of muscle cells obtained from a subject with a myopathy, or neurodegenerative disease etc., as well to monitor the morphological or differentiative behavior of myocytes that are electrically connected to neurons from diseases subjects in vitro. Certain treatments can induce changes in morphological behavior of muscle cells, such as inducing hypertrophy which is associated with myocyte elongation and expansion. Because impedance monitoring can detect changes in cell morphology, it can be used to for detection of hypertrophy in myocytes.

In myocyte culture preparation, impedance monitoring reflects the cell growth, which can be correlated through an impedance-based curve, namely, a cell index curve and contraction/relaxation function by impedance beating signals; while extracellular recording reflects myocyte excitation and conduction by field potential signals. Further, the device described herein provides the ability to monitor viability of myocytes after specific electrical activation by distinct neuronal populations, as well as the field potential of myocytes, or the excitation-contraction coupling or beating and morphological and differentiative aspects of myocytes in a label-free manner and real-time manner.

While the devices are primarily discussed in regards to motor neurons and myocytes, and their connection via NMJs, the devices can be used to assess other neuronal cell types, e.g., sympathetic neurons and sensory neurons, as well as other myocytes populations, e.g., the cardiomyocytes and sympathetic neuron interactions.

Electro-stimulation of the neuronal cells attached to the first cell growth area is accomplished by an electrical signal delivered from the power source to the field potential electrodes and to attached cells, which can be in the form of a series of electrical pulses. That is, while impedance monitoring and may incorporate different spectra, waveforms or the like that can continue over an extended time, electro-stimulation of the neurons via the field potential electrodes is typically performed more quickly in a fast on-off approach followed by a delay in the off state to permit measurement of the excitable cells without interference of the electro-stimulation signal. The skilled artisan will appreciate that in some instances the electro-stimulation signal may be at least partially filtered to prevent interference from a lower intensity state. Preferably, electro-stimulation of the neuronal cells attached to the first cell growth area is performed at a plurality of time intervals. Most preferably, the time intervals are at regular time intervals. As a non-limiting example, electro-stimulation can be performed by delivering a biphasic pulse of about 0.8 ms-1.5 ms, more preferably about 1 ms and at an output voltage of 1V-2 V or more preferably about 1.2 V.

In some embodiments, an extracellular recording amplifier is communicatively coupled to the impedance electrodes for electrical communication to permit amplifying and recording electrical voltage signals of the ground electrodes.

In some embodiments, the plurality of neuronal cells is derived from iPSCs obtained from a healthy subject or a subject with a neurodegenerative disease or disorder or a myopathy. In some embodiments, the plurality of muscle cells is derived from iPSCs obtained from a healthy subject or a subject with a neurodegenerative disease or disorder or a myopathy.

In some embodiments, the plurality of neuronal cells comprises motor neurons. In some embodiments, the plurality of muscle cells comprise skeletal muscle cells.

In some embodiments, the plurality of neuronal cells comprises sympathetic neurons. In some embodiments, the plurality of muscle cells comprises smooth muscle cells.

In some embodiments, the plurality of neuronal cells comprises sympathetic neurons. In some embodiments, the plurality of muscle cells comprises cardiac muscle cells or cardiomyocytes.

In some embodiments, the plurality of neuronal cells, or plurality of muscle cells, or both, are genetically modified cells to introduce one or more mutations for a neurodegenerative disease or myopathy.

In some embodiments, the plurality of neuronal cells, or plurality of muscle cells, or both, are isogenic controls of a genetically modified cell that has one or more mutations introduced for a neurodegenerative disease or myopathy.

As used herein, “isogenic control” refers to a cell that is genetically identical (or substantially genetically identical) to a genetically modified cell, except for the genetic modification. In some embodiments, an isogenic control can be a (e.g., healthy control) cell prior to the introduction of (e.g., disease-associated) mutation(s). In some embodiments, an isogenic control can be a cell genetically modified from a patient sample cell, wherein the disease-associated mutation(s) has been genetically modified to the heathy genotype.

In some embodiments, methods as described herein can be used to assess electrical conductance across at least one neuromuscular junctions (NMJ) between an axon extended from the first cell type and the cell bodies of the second cell type.

In another aspect described herein are MEA devices and methods for using these devices including, for example, in the study of cells and tissue. In accordance with the MEA devices, cells can be cultured more naturally, permitting the cultured cells and tissue to be studied in-vitro, but in a more natural context. In accordance with some embodiments, the MEA devices include a cell culture surface upon which the cells and tissue to be studied can be cultured. The cell culture surface can include micrometer and nanometer sized features that encourage the cells and tissue to culture in configurations that more closely model the way the cells and tissues would develop in the body, such as when using an extracellular matrix (ECM).

In some embodiments, the electrode arrays can be used to stimulate and record signals from cells cultured on the cell culturing surface. In some embodiments, a signal generator can be used to provide electrical input to the electrode arrays which are contact the cultured cells. In alternative embodiments, the electrode arrays can be used to record from the cells cultured on the cell culturing surface. A whole range of biopotentials and physico-chemical properties can be recorded from the cells cultured on the MEA device, e.g., but not limited to, functional parameters, including, but not limited to action potential duration (ADP), wave propagation, action potential frequency, beat frequency, action potential transmission, Vmax of the action potential, contraction force, peal to peak amplitude, end diastole to peak diastole rate and the like.

Neuronal and muscle cells may be cultured on the first and second cell growth area respectively, then immediately stimulated or measured; however, in most embodiments, it is preferable to culture cells for a sufficient amount of time such that they attach to the substrate, and extend axons across the axon outgrowth area prior to stimulation and impedance measuring. More preferably, neuronal cells and myocytes can be grown or differentiated on the first and second cell growth area respectively for a sufficient time in that they join to form a cell layer over the field potential electrodes, and the impedance electrodes, respectively. In some embodiments, electro-stimulation is performed within 1 hour of placing cells in the device; however, the nanotextured substrate has been shown suitable for long term culturing and thus cells may be cultured as desired, such as for more than 5 days, more than 10 days, more than 15 days, more than 20 days, more than 1 month, more than 2 months, more than 3 months or more than 200 days in vitro, even more than one month depending on the interests of the experiment and regular cell culture maintenance.

Further, impedance monitoring may be used to assess how well muscle cells are attached to the substrate, the viability of the cells or to assess the quantity of cells in the sample prior to electro-stimulation, to assess growth curves or the like. Further, impedance recording may be used to determine whether electro-stimulation is necessary or desired. In some instances, cardiomyocytes derived from embryonic stem cells will begin to beat spontaneously, which can be detected or measured through impedance and extracellular recording. However, even cells that being spontaneous beating can be electro-stimulated via FPE stimulation of the neurons and the neuronal-muscle connectivity via the axons to establish a more regular beating or a beating at an increased frequency.

Neuronal cells are electro-stimulated through applying a signal via the plurality of field potential electrodes. While the particular specifications of a device can vary, stimulation is generally accomplished by applying voltage in a range of about 1 V-2 V for about 1 ms, at an interval of every 1 second-1.5 seconds or so. In some embodiments, the voltage applied is 1.1 V for 1 ms every 1.2 s-1.3 s.

In some embodiments, the field potential electrodes can be used to electro-stimulate the neurons simultaneously or in pulses, which can be staggered. Electro-stimulation intervals can be selected through a software and computer interface. In some embodiments, the field potential electrical stimulation of the neurons can be simultaneous with the impedance monitoring of the muscle cells, or it can be staggered, that is the field potential electrodes electro-stimulate the neurons at time period before the impedance monitoring of the muscle cells.

When performing impedance monitoring, preferably, impedance is measured at millisecond time resolution. For example, resolution on the order of 1 ms-10 ms provides high resolution of, e.g., beating of cardiomyocytes, or e.g., the contraction of skeletal muscle cells. Impedance measurements may themselves be compared; however, in preferred embodiments a cell index is calculated from impedance measurements as an impedance parameter and plotted over time as an impedance-based curve or cell index curve. Accordingly, a cell index curve may be used as an impedance-based curve for comparison, such as by comparing cell index curves over time. In other embodiments, cell change index is calculated from cell index and plotted as an impedance-based curve. Accordingly, cell change index may be used as impedance-based parameter for comparison, such as by comparing cell change index curves over time. The skilled artisan will appreciate that when comparing impedance-based curves, a same type of curve will be compared. For example, comparison would involve comparing cell index curves to one another, cell change index curves to one another and the like.

While impedance measurements themselves can be compared, their comparison is complicated by culture conditions, such as differences between cell populations. Accordingly, a number of calculations have been established previously that result in values that permit improved comparison. Among these are cell index (CI) and cell change index (CCI), each of which has been discussed in detail in the following U.S. Pat. Nos. 7,470,533, 7,459,303, 7,192,752, 8,026,080, 7,560,269, 8,263,375, 7,468,255, 8,206,903 and 8,420,363, which are incorporated by reference herein in their entireties. Accordingly, the use of cell index and cell change index in the formation of an impedance-based curve is well established in the art. Further, these documents can be consulted for method of calculating cell index (CI) or cell change index (CCI).

The cell index (CI) obtained for a device reflects how many muscle cells are attached to the electrode surfaces in this well and how well muscle cells are attached to the electrode surfaces in the second cell growth area. In this case, a zero or near-zero “cell index” indicates that no cells or very small number of cells are present on or attached to the electrode surfaces. In other words, if no cells are present on the electrodes, or if the cells are not well-attached onto the electrodes cell index=0. A higher value of “cell index” indicates that, for same type of the cells and cells under similar physiological conditions, more cells are attached to the electrode surfaces. Thus, Cell Index is a quantitative measure of cell number present in a well. A higher value of “cell index” may also indicate that, for same type of the cells and same number of the cells, cells are attached better (for example, cells spread out more, or cell adhesion to the electrode surface is stronger) on the electrode surfaces.

In other embodiments, a normalize cell index is calculated from the cell index and plotted as an impedance-based curve. A “normalized cell index” at a given time point is calculated by dividing the Cell Index at the time point by the Cell Index at a reference time point. Thus, the Normalized Cell Index is 1 at the reference time point. Normalized cell index is cell index normalized against cell index at a particular time point. In most cases in the present applications, normalized cell index is derived as normalized relative to the time point immediately before a compound addition or treatment. Thus, normalized cell index at such time point (immediately before compound addition) is always unit one for all wells. One possible benefit for using such normalized cell index is to remove the effect from difference in cell number in different wells. A well having more cells may produce a larger impedance response following compound treatment. Using normalized cell index, it helps to remove such variations caused by different cell numbers.

In other embodiments, a cell change index (CCI) is calculated from the cell index. A “cell change index” at a given time point is calculated by subtracting the cell index at a standard time point from the cell index at the given time point. Thus, the cell change index is the absolute change in the cell index from an initial time (the standard time point) to the measurement time. CCI is the normalized rate of change in cell index. CCI values can be used to quantify the cell status change. For cells in an exponential growth under regular cell culture condition, the cell index determined by a cell-substrate impedance monitoring system described herein is expected to be a proportionate measure of the cell number in the well since the cell morphology and average extent of cell adhesion to the electrode surfaces among the whole cell population do not exhibit significant changes over time.

Turning back to comparing impedance-based parameters or impedance-based curves, in some embodiments, cell index is preferably calculated and plotted over time to form an impedance base curve. Impedance based curves over time may be aligned or overlaid with one another according to electro-stimulation time points. For example, two or more impedance-based curves may be aligned or overlaid using a point or time of electro-stimulation as a starting basis.

A skilled artisan will appreciate that when comparing impedance-based parameters each member for comparison is a member of a same parameter. That is, impedance measurements can be compared to one another; cell index, which can be calculated from the impedance measurement, can be compared to one another; or cell change index, which can be calculated from cell index, can be compared to one another. Although individual members of a parameter can be compared, preferably the impedance based parameters are plotted over time to provide an impedance-based curve then compared, whether an impedance measurement curve, a cell index curve, or cell change index curve to identify differences changes in impedance, which may be associated with administration of a compound, expression of an inserted nucleic acid or the like. In preferred embodiments, cell index is calculated from impedance measurements and plotted over time to provide an impedance-based curve in the form of a cell index curve.

Extracellular recording measures the extracellular potential (also referred to as field potential (FP)) and is a close simulation to its counterpart, intracellular action potential (AP), therefore identifying peaks and wave form changes in field potential can be used to predict ion channel activity. Accordingly, the extracellular recording measurements are plotted over time, typically in microvolts, to identify variations in the field potential (FP). Sampling rate for extracellular recording can be about 1 KHz, 2 KHz, 5 KHz and 10 KHz. Extracellular potential is preferably plotted over time to form a field potential curve.

Periods or electro-stimulation intervals from two or more impedance curves and/or two or more field potential curves may be overlaid to identify trends or differences in impedance and/or field potential. For clarity, the two or more impedance curves can be from different wells, such as from wells having serial dilutions of a compound or may be from a same well at a later time point. Accordingly, the addition of compounds suspected of affecting either the field potential (likely due to affecting ion channels) or impedance of the excitable cells may be added to a culture of cells and changes in impedance or field potential can be identified or measured through the comparison of the impedance-based parameter and field potential before and after compound addition, such as by comparing cell index curves or field potential curves over time. Accordingly, in preferred embodiments comparisons are performed by comparing cell index curves over time to one another and comparing field potential curves over time to one another. In instances where it is not readily apparent by viewing each curve over time, curves between different electro-stimulation periods can be overlaid and compared using curve comparison algorithms. Accordingly, overlaying impedance curves may identify changes in impedance and overlaying field potential curves may identify changes in field potential.

The methods, devices and systems as disclosed are useful in assessing the toxicity of diseased neuronal cells (e.g., neuronal cells derived from a subject with a neurodegenerative disease, myopathy, or neuromuscular disease) on muscle cells, or vice versa, the toxicity of diseased muscle cells (e.g., neuronal cells derived from a subject with a neurodegenerative disease, myopathy, or neuromuscular disease) on neurons. The methods, devices and systems as disclosed herein can be used for assessing toxicity of compounds on either the neuronal cells, or muscle cells, or both, through monitoring impedance of a beating muscle population and identifying any changes after compound administration. For instance, a compound suspected of affecting excitation contraction coupling of the muscle cells can be provided to either the neurons, or the muscle cells, or both. As the device permits the spatial separation of the neuronal and muscle cell populations, yet allows them to electrically connect via axonal projections, it allows the assessment of the effect of the compound on the electro-conductivity properties of each cell type individually. That is, without the spatial separation of the neuronal and muscle cell populations, one would not be able to assess the effect of the compound on the electro-conductivity properties of each cell type individually. Field potential electrostimulation of the neurons, and impedance monitoring of the muscle cells can be performed before and after adding the compound and impedance-based parameters (such as cell index) prior to and after adding the compound can be calculated and compared to identify changes in the impedance parameter in response to the compound and thus predict whether the compound is likely to be toxic to the neurons, muscle cells or both. Preferably, the impedance-based parameter is compared between at least two different electro-stimulation intervals; however, three or more, four or more, five or more, six or more and the like intervals can be compared.

In some embodiments, impedance is conducted to assess an effect of a compound, such as its potential toxicity, on cell. An exemplary method would be to provide a population of neuronal cells to the first cell growth area, a population of muscle cells to the second cell growth area culture, culture for a sufficient amount of time to allow the neurons to extend axons into the axon outgrowth area and to from NMJ or other connections with the muscle cells, electro-stimulate the neuronal cells located on the first cell growth area, perform impedance monitoring recording of the muscle cells located on the second cell growth area before and after adding the compound and analyzing the results. As such, the analysis could include calculating an impedance-based parameter (such as cell index or cell change index) from impedance prior to and after adding the compound and their comparison to identify changes in the impedance parameter. Further, the impedance-based parameter may be plotted over time to form an impedance-based curve before and after administration for comparison. Alternatively or in addition, field potential of the neuronal cells can also be assessed before electrostimulation, and also before and after administration can be compared such as by comparing field potential curves over time to identify differences. Such analysis in response to the compound can be used to predict whether the compound is likely to be toxic to the neurons, or muscle cells, or both, or if it affects beating, affects myocyte morphology or affects ion channels. Preferably, the impedance-based parameter is compared between at least two different electro-stimulation intervals; however, three or more, four or more, five or more, six or more and the like intervals can be compared. Compounds for testing are not intended to be limiting and may include organic or inorganic molecules, drugs, peptides, proteins, antibodies, siRNA, shRNA, miRNA, cDNA, lipids or any combination thereof.

While the above has been primarily discussed with respect to monitoring the excitable cells before and after adding a compound suspected of having a toxic affect to a well and comparison of impedance or field potential before and after administration, in related approaches the compound can be added to two or more wells in different concentrations to evaluate toxicity, e.g., myotoxicity, cardiotoxicity or neurotoxicity, or activity compared to dose or can be added to two or more devices in an array and administered in same well with an antagonist, such as to further verify suspected findings or mechanism of action. For instance, impedance-based curves can be used to calculate the compound dose-dependent changes in myocyte morphology, ion channel modulation, impedance, field potential or the like and generate an EC-50 value for the potency of the compound. In addition, extracellular recording permits comparisons with cells at various developmental stages to assess development.

### B. Assays

In one aspect, described herein is an assay for measuring the effect of an agent on electroconductive cells, the assay comprising contacting a cell culture system as described herein with the agent and measuring the growth, viability, or activity of the electroconductive cells. The agent can be a drug or drug candidate and the assay can assess the safety of the drug with regards to electroconductive cells. In some embodiments, the agent can be a drug or drug candidate for a cardiovascular, muscular, or neuronal disease or disorder.

Accordingly, in one aspect described herein is an assay for assessing an agent for modulation of electrical signaling from one cell type to another cell type, comprising: providing a device as described herein, wherein the device comprises a first cell type located on surface of the first cell growth area, and a second cell type located on the second cell growth area, and wherein the first cell type extends axons across the axon outgrowth area from the first cell area to the second cell type in the second cell growth area; contacting the first cell type, second cell type, or both, with an agent; providing electrical stimulation to the first cell type via the field potential electrodes; recording electrical activity of the second cell type via the impedance electrodes; and detecting a change in electrical activity of the second cell type recorded via the impedance electrodes in the presence of the agent as compared to the absence of the agent.

In some embodiments, the agent can be known neuromuscular synaptic agonists and antagonists (see e.g., Table 2). In some embodiments, the agent can be a drug to treat a neurodegenerative disease or disorder or a myopathy (e.g., Mexiletine, cyclooxygenase-2 inhibitors, tramadol, corticosteroids, seizure medications such as gabapentin or pregabalin, antidepressants such as amitriptyline, Cymbalta (a serotonin norepinephrine reuptake inhibitor) and the like). In some embodiments, an agent can be tested using an assay as described herein for efficacy in treating a neurodegenerative disease or disorder or a myopathy (e.g., ALS, CMT).

In some embodiments, the electrical activity detected can comprise contraction magnitude, contraction velocity, and relaxation speed of the second cell type (e.g., muscle cells). In some embodiments, a change can comprise a significant increase compared to a control. In some embodiments, a change can comprise a significant increase compared to a control.

In some embodiments, the assay can further comprise determining that an agent is effective for treating a neurodegenerative disease or disorder or a myopathy if it modulates the electrical activity of cells derived from a subject with a neurodegenerative disease or disorder or a myopathy, e.g., to exhibit more similar electrical activity to an isogenic control or healthy control.

In some embodiments, the MEA device comprising electroconductive cells can be used in methods and assays for toxicity screening, e.g., to assess the effect of an agent on the biopotentials of the cultured cells, e.g., to identify any agent which adversely effects the biopotential of the cultured cells.

In some embodiments, the MEA device comprising electroconductive cells can be used in drug screening methods and assays, e.g., to assess the effect of potential therapeutic agents for the treatment of a disease or disorder. In some embodiments, for example, where the cultured cells are derived from a subject with a neurodegenerative disease or disorder (e.g., peripheral neuropathy) or a myopathy (or for example cardiovascular disease or disorder, or a subject having arrhythmia) the MEA comprising electroconductive cells can be used to screen for, and identify agents which restore, either partially or completely, the biopotential and electroconductive properties of the cells back to a more normal phenotype. In such embodiments, the cells can be human cells, which are originally derived from iPSC and differentiated into electroconductive cells, e.g., neuronal and/or muscle cells as described herein.

In some embodiments, the MEA device comprising electroconductive cells can be used for disease modeling, e.g., to see the effect of a particular transient electric signal on the phenotype of the electroconductive cells. In some embodiments, the phenotype can be the morphology or cell characteristics (e.g., expression of particular markers) or the chemo-electric properties of the cell.

In alternative embodiments, the MEA device can be used to assess the effect of different electrical signals on the development, differentiation, maturation, functionality and/or survival of electroconductive cells. For example, one can use the MEA device to provide electrical signals to the cells to induce them to differentiate along a particular lineage, e.g., to differentiate stem cells into an electroconductive cell type, e.g., neuronal and muscle cells, and subtypes thereof, including cardiomyocytes, skeletal myocytes and the like.

Similarly, in some embodiments, one can use the MEA device to provide electrical signals to immature electroconductive cells to enhance their maturation to a more mature phenotype, e.g., by way of an example only, the MEA can be used to enhance the maturation of immature skeletal muscle cells to more mature skeletal muscle cells, e.g., with characteristics of mature adult skeletal muscle cells found in vivo. As another example, the MEA can be used to enhance the maturation of immature cardiomyocytes to more mature cardiomyocytes, e.g., with characteristics of mature adult cardiomyocytes found in vivo. As another example, the MEA can be used to enhance the maturation of immature smooth muscle cells to more mature smooth muscle cells, e.g., with characteristics of mature smooth muscle cells found in vivo.

In some embodiments, the MEA device comprising electroconductive cells can be used in the identification of changing action potentials in muscle cells (e.g., skeletal muscle cells, cardiomyocytes, smooth muscle cells), and thus can be used in the diagnosis of a myopathy or a cardiovascular disease or disorder, including, but not limited to arrhythmia in a subject. In some embodiments, where the electroconductive cells are skeletal muscle cells, the MEA device comprising skeletal muscle cells can aid the diagnosis of neuromuscular diseases and disorders, and/or myogenic disorders such as ALS, muscular dystrophy and the like. Similarly, the MEA device comprising electroconductive cells can be used in the identification of changing action potentials of neuronal cells, and thus can be used in the diagnosis of neurological and/or disorders and neurodegenerative disorders, e.g., including but not limited to, Parkinson's disease, Huntington's disease, ALS, Alzheimer's disease. In such embodiments, the electroconductive cells can be differentiated from cells obtained from the subject, e.g., electroconductive cells derived from iPSC originally obtained from the subject. In some embodiments, electrocardiography depends on measurement. Electromyography and electroencephalography function similarly in the diagnosis of neuromuscular and brain disorders, respectively.

In another aspect, described herein is an assay for measuring the effect of an agent on electroconductive cells, the assay comprising contacting a cell culture system as described herein with the agent and measuring the growth, viability, or activity of the electroconductive cells. The agent can be a drug or drug candidate and the assay can assess the safety of the drug with regards to electroconductive cells. In some embodiments, the agent can be a drug or drug candidate for a cardiovascular, muscular, or neuronal disease or disorder.

Generally, agents can be tested at any concentration that can modulate the activity, growth, and/or viability of an electroconductive cell. In some embodiments, agents are tested at concentration in the range of about 0.1 nM to about 1000 mM. In one embodiment, the compound is tested in the range of about 0.1 μM to about 20 μM, about 0.1 μM to about 10 μM, or about 0.1 μM to about 5 μM. Depending upon the particular embodiment being practiced, the candidate or test agents can be provided free in solution. Additionally, for the methods described herein, test agents can be screened individually, or in groups. Group screening is particularly useful where hit rates for effective test agents are expected to be low such that one would not expect more than one positive result for a given group.

Methods for developing small molecule, polymeric and genome-based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule (Woburn, Mass.), Invitrogen (Carlsbad, Calif.), Ryan Scientific (Mt. Pleasant, S.C.), and Enzo Life Sciences (Farmingdale, N.Y.). These libraries can be screened for the ability of members to modulate the activity, growth, and/or viability of electroconductive cells using e.g., methods described herein.

In some embodiments, the agents can be naturally occurring proteins or their fragments. Such agents can be obtained from a natural source, e.g., a cell or tissue lysate. Libraries of polypeptide agents can also be prepared, e.g., from a cDNA library commercially available or generated with routine methods. The agents can also be peptides, e.g., peptides of from about 5 to about 30 amino acids, with from about 5 to about 20 amino acids being preferred and from about 7 to about 15 being particularly preferred. The peptides can be digests of naturally occurring proteins, random peptides, or “biased” random peptides. Peptide libraries, e.g., combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The agents can also be nucleic acids. Nucleic acid candidate agents can be naturally occurring nucleic acids, random nucleic acids, or “biased” random nucleic acids. For example, digests of prokaryotic or eukaryotic genomes can be similarly used as described above for proteins.

The agent can function directly in the form in which it is administered. Alternatively, the agent can be modified or utilized intracellularly to produce a form that modulates the desired activity, e.g., introduction of a nucleic acid sequence into a cell and its transcription resulting in the production of an activity within the cell.

In some embodiments, the agent that is screened and identified to modulate the activity, growth, and/or viability of an electroconductive cell using the methods described herein by at least 5%, preferably at least 10%, 20%, 30%, 40%, 50%, 50%, 70%, 80%, 90% relative to an untreated control. A level which is higher or lower than a reference level (e.g., the level in the absence of the agent) can be a level which is statistically significantly different than the reference level. In some embodiments, a level that is lower than a reference level can be 90% or less of the reference level, e.g., 90% or less, 80% or less, 70% or less, 60% or less, 50% or less, 25% or less, or 10% or less of the reference level. In some embodiments, a level that is higher than a reference level can be 1.5× or more of the reference level, e.g., 1.5× or more, 2× or more, 3× or more, 5× or more, or 10× or more of the reference level. The reference level can be the level in the absence of the agent, e.g., the level in a parallel, untreated cell culture well (e.g., of the same multi-well microelectrode array), the level in the cell and/or well prior to contact with the agent, and/or a level in a population of cells not contacted with the agent, e.g., a pre-determined level.

In some embodiments, the methods and assays described herein can relate to measuring or determining cell viability, e.g., after contacting electroconductive cells with an agent. “Measuring cell viability” refers to measuring or detecting any aspect of cell metabolism, growth, structure, and/or propagation which is indicative of either a healthy, viable cell or a dead and/or nonviable cell. Colorimetric, luminescent, radiometric, and/or fluorometric assays known in the art can be used. In some embodiments, determining cell viability can comprise manual counting of cells using a hemocytometer. In some embodiments, determining cell viability can comprise the use of a live-dead cell stain, e.g., a stain which will stain either a live cell or a dead cell.

Colorimetric techniques for determining cell viability include, by way of non-limiting example, Trypan Blue exclusion. In brief, cells are stained with Trypan Blue and counted using a hemocytometer. Viable cells exclude the dye whereas dead and dying cells take up the blue dye and are easily distinguished under a light microscope. Neutral Red is adsorbed by viable cells and concentrates in cell lysosomes; viable cells can be determined with a light microscope by quantitating numbers of Neutral Red stained cells.

Fluorometric techniques for determining cell viability include, by way of non-limiting example, propidium iodide, a fluorescent DNA intercalating agent. Propidium iodide is excluded from viable cells but stains the nucleus of dead cells. Flow cytometry of propidium iodide labeled cells can then be used to quantitate viable and dead cells. Release of lactate dehydrogenase (LDH) indicates structural damage and death of cells, and can be measured by a spectrophotometric enzyme assay. Bromodeoxyuridine (BrdU) is incorporated into newly synthesized DNA and can be detected with a fluorochromelabeled antibody. The fluorescent dye Hoechst 33258 labels DNA and can be used to quantitate proliferation of cells (e.g., flow cytometry). Quantitative incorporation of the fluorescent dye carboxyfluorescein diacetate succinimidyl ester (CFSE or CFDA-SE) can provide cell division analysis (e.g., flow cytometry). This technique can be used either in vitro or in vivo. 7-aminoactinomycin D (7-AAD) is a fluorescent intercalator that undergoes a spectral shift upon association with DNA, and can provide cell division analysis (e.g., flow cytometry).

Radiometric techniques for determining cell proliferation include, by way of non-limiting example, the use of [3H]-Thymidine, which is incorporated into newly synthesized DNA of living cells and frequently used to determine proliferation of cells. Chromium (51Cr)-release from dead cells can be quantitated by scintillation counting in order to quantitate cell viability.

Luminescent techniques for determining cell viability include, by way of non-limiting example, the CellTiter-Glo luminescent cell viability assay (Promega Madison Wis.). This technique quantifies the amount of ATP present to determine the number of viable cells.

Kits for determining cell viability are commercially available, e.g., the MUTLITOX-FLOUR™ Multiplex Cytotoxicity Assay (Cat. No. G9200; Promega, Inc.; Madison, Wis.). In some embodiments, the means of determining cell viability can comprise a high-throughput method, e.g., live-dead cell stains can be detected using a fluorescence-capable multiplate reader. In some embodiments, imaging analysis can be performed via automated image acquisition and analysis.

Measurements of cell growth can include, e.g., measurements of changes in cell volume and/or size and/or measurement of cell proliferation. Changes in cell size can be measured, e.g., using imaging analysis software. Cell proliferation can be measured using, e.g., an MTS assay commercially available from a variety of companies including RnD Systems, and Promega, among others.

In some embodiments, the activity of the electroconductive cells can be an activity selected from the group consisting of field potential; field potential profiles; field potential duration; QT interval length, conduction velocity; electrophysical profile; spontaneous beating rate; reentrant wave patterns; and impedance.

In some embodiments, the assay can further comprise inducing arrhythmia in the cells and the measuring the ability of the agent to restore normal field potential properties. In some embodiments, the activity of the agent as an anti-arrhythmia agent is measured. Arrhythmia can be induced in the cells comprised by a cell culture system described herein by a number of methods. In some embodiments, arrhythmia can be induced by contacting the cells with an arrhythmia-inducing agent. Such agents are known in the art and can include, by way of non-limiting example, epinephrine or norepinephrine. In some embodiments, arrhythmia can be induced by providing irregular electrical stimulation to the cells via the multi-electrode array, e.g., an irregular pattern of electrical impulses, varying in magnitude and/or frequency. In some embodiments, arrhythmia can be induced by culturing the cells on a multi-electrode array having irregular nanogrooves. In some embodiments, arrhythmia can be induced by culturing the cells on a multi-electrode array in which a portion of the cell culture surface comprises irregular nanogrooves. In some embodiments, a combination of arrhythmia-inducing methods can be combined.

### C. Systems

In one aspect described herein is a system for measuring the electrical conductance from one cell type to a second cell type comprising: a device as described herein, an electronic interface for stimulation of field potential electrodes on the device, and an electronic interface that permits recording of electrical activity from the impedance electrodes. In some embodiments, an electrical interface can be provided that permits recording of electrical activity of field potential electrodes on the device, and it can be the same or different interface that permits recording of electrical activity from the impedance electrodes.

In some embodiments, the MEA device can be part of a system, e.g., where the MEA device is connected to an electronic interface (e.g., signal generator and/or signal recorder), which can be optionally connected to a computer, where the computer instructs the electronic interface, whereby the computer directs the transmission of electric signals to and from the MEA device. In some embodiments, the MEA device of the system comprises electroconductive cells.

An exemplary system for the device requires electrical connection of the device to a suitable a suitable electro-stimulation power source, impedance analyzer, and/or extracellular recording amplifier. Accordingly, one aspect provides a system for monitoring neuronal-muscle electrical interaction, which includes the MEA device as disclosed herein; a power source configured to deliver an electrical signal capable of electro-stimulating the neuronal cells located on the first cell growth area via a plurality of field potential electrodes; and at least one analyzing module for measuring an electrical property from muscle cells located on the second cell growth area via the plurality of impedance electrodes, the field potential electrodes configured to receive a signal from the power source to thereby delivering an electro-stimulating signal to the neurons attached to the first cell growth area of the substrate; and the impedance electrodes communicatively coupled to the at least one analyzing module, thereby permitting impedance monitoring of the muscle cells attached to the second cell growth area on the substrate.

Generally, it is desirable and it is recognized for the ground electrodes to have small electrode impedances. The small electrode impedance is achieved by using ground electrodes with large effective surface areas by increasing the ratio of the surface area of the ground electrodes to that of impedance or field potential electrode by a factor of a hundred, even thousands of times. Such small electrode geometry has advantages of recording the electrical potential generated by a small number of the cells located on the recording electrodes. Action potentials from such a small number of the cells tend to be synchronized or nearly synchronized, allowing for a better time resolution for recording impedance and for resolving different features of the recorded potential.

In the system for monitoring impedance of muscle cells the impedance analyzer is communicatively coupled to impedance monitoring electrodes to monitor impedance. In some embodiments, the impedance analyzer is capable of measuring impedance between 0.1 ohm and 105 ohm in frequency range of 1 Hz to 1 MHz. The impedance analyzer is preferably capable of measuring both resistance and reactance (capacitive reactance and inductive reactance) components of the impedance. In one embodiment of the above system, the impedance analyzer is capable of measuring impedance between 1 ohm and 103 ohm in frequency range of 1.00 Hz to 300 kHz.

In some embodiments, the impedance analyzer is capable of impedance measurements at millisecond time resolution. The required or desired time resolution may vary depending on the excitation cycle of the muscle cell. Muscle cells having shorter excitation cycles or pacing electro-stimulation more quickly, would tend to require faster time resolution. In some embodiments 500 millisecond time resolution is sufficient, such that at least two consecutive impedance measurements are between about 300 milliseconds and about 500 milliseconds apart. In preferred embodiments, impedance measurement at millisecond time resolution includes at least two consecutive impedance measurements less than 100 milliseconds apart. In some instances, the at least two consecutive impedance measurements are less than 50 milliseconds or less than 40 milliseconds apart. In some instances, the at least two consecutive impedance measurements are less than 20 milliseconds apart. In some instances, at least two consecutive impedance measurements are less than 10 milliseconds apart. In some instances, millisecond time resolution includes two consecutive impedance measurements between 1 millisecond and 5 milliseconds, between 5 milliseconds and 10 milliseconds, between 10 milliseconds and 20 milliseconds, between 20 milliseconds and 40 milliseconds, or between 40 milliseconds and 50 milliseconds apart. In some instances, millisecond time resolution includes at least two consecutive impedance measurements between 50 milliseconds and 100 milliseconds apart. In some instances, millisecond time resolution includes at least two consecutive impedance measurements between 100 milliseconds and 150 milliseconds or between 150 and 300 milliseconds apart.

Achieving millisecond time resolution can be achieved by using fast processing electronic chips for analogue-to-digital conversion, for parallel digital signal processing and data calculation with field-programmable gate array (FPGA) and for fast communication between the impedance measurement circuitry and software. Another example includes the use of multiple analogue-to-digital (AD) conversion channels so that analog electronic signals from multiple channels can be converted to digital signals simultaneously. Such parallel AD conversion is important, particular for the system having multiple wells, the measurement time resolution of each is required to be in the millisecond resolution.

In addition, when using a device in an array, the software can issue a command for measuring multiple devices' impedances. The measurement circuitry would simultaneously or nearly simultaneously perform signal conversion, signal processing and impedance calculation for multiple wells. The multiple impedance data for the multiple devices would be sent over the communication lines to the computer sequentially with one device's data at the same time or simultaneously with more than one device's data being sent at a time. In this “measurement of multiple-devices' impedances at a time” mode, the system may be performing multiple tasks simultaneously, for example, while one device's impedance data is being measured and calculated, another device's impedance data may be communicated and sent over the communication lines to the computer.

With millisecond time resolution for impedance measurement, it becomes possible to resolve, for example, individual contraction cycles of myocytes, or individual beating cycles of cardiomyocytes, cultured on electrodes and study the excitation, contraction and release of contracting or beating cells. Whilst theoretically one needs at least two data points for each contraction cycle or beating cycle, in practice more than 2 data points are needed for each contraction cycle or beating cycle. For example, if cells have a contraction or beating rate of 60 beats per minute, i.e., one contraction per second or beat per second, it would be preferred to have a time resolution of at least 200 milliseconds so that each contraction cycle or beating cycle consists of 5 data points. More preferably, the measurement time resolution is 100 milliseconds. Still more preferably, the time resolution is 50 milliseconds or less.

Cell-substrate impedance monitoring at millisecond time resolution can be used to efficiently and simultaneously perform multiple assays by using circuitry of the device station to digitally switch from recording from measuring impedance over an array in one well to measuring impedance over an array in another well. Similarly, groups of wells may be monitored simultaneously and switching between occur between designated groups. In one embodiment of the above system, the system under software control is capable of completing an impedance measurement for an individual well at a single frequency within milliseconds, such as less than 100 milliseconds, less than 40 milliseconds, less than 20 milliseconds, less than 10 milliseconds or between 1 millisecond and 40 milliseconds. In some embodiments the user may choose the frequency of measurement for millisecond time resolution.

It should initially be understood that the disclosure herein may be implemented with any type of hardware and/or software, and may be a pre-programmed general-purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present technology as disclosed herein, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### D. Kits

In one aspect, provided herein is a kit comprising a multi-electrode array as described herein. In some embodiments, the kit can further comprise at least one electroconductive cell or at least two different types of electroconductive cells (e.g., neuronal cells and muscle cells). In some embodiments, provided herein is a kit comprising a cell culture system as described herein.

A kit is any manufacture (e.g., a package or container) comprising at least one multi-electrode array according to the various embodiments herein, the manufacture being promoted, distributed, or sold as a unit for performing the methods or assays described herein. The kits described herein include reagents and/or components that permit the culture of electroconductive cells and/or the recording and/or stimulation of electrical signals associated with those cells. The kits described herein can optionally comprise additional components useful for performing the methods and assays described herein. Such reagents can include, e.g., cell culture media, growth factors, differentiation factors, buffer solutions, labels, imaging reagents, and the like. Such ingredients are known to the person skilled in the art and may vary depending on the particular cells and methods or assay to be carried out. Additionally, the kit may comprise an instruction leaflet and/or may provide information as to the relevance of the obtained results.

Another aspect of the technology described herein relates to kits comprising a multi-electrode array as described herein. Described herein are kit components that can be included in one or more of the kits described herein.

In some embodiments, the kit comprises an effective amount and/or an effective number of reagents (e.g., cell culture reagents). As will be appreciated by one of skill in the art, reagents can be supplied in a lyophilized form or a concentrated form that can diluted prior to use with cultured cells. Preferred formulations include those that are non-toxic to the cells and/or does not affect growth rate or viability etc. reagents can be supplied in aliquots or in unit doses.

In some embodiments, the components described herein can be provided singularly or in any combination as a kit. In addition, the kit optionally comprises informational material. In some embodiments, the kits provided herein comprise an aliquot of reagents and, optionally, an aliquot of at least one cell type. The kit can also contain a substrate for coating culture dishes, such as laminin, fibronectin, Poly-L-Lysine, or methylcellulose.

In some embodiments, the compositions in the kit can be provided in a watertight or gas tight container which in some embodiments is substantially free of other components of the kit. For example, a reagent can be supplied in more than one container, e.g., it can be supplied in a container having sufficient reagent for a predetermined number of reactions, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of reagents, concentration, date of expiration, batch or production site information, and so forth. In one embodiment, the informational material relates to methods for using or administering the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### V. Neurodegenerative Disease or Disorder or a Myopathy

Described herein are devices, systems, methods, and assays that are used to study disorders including neurodegenerative disease or disorder (e.g., peripheral neuropathy) or a myopathy. Peripheral neuropathies are a clinically and genetically heterogeneous set of severely debilitating neurological conditions with common symptoms being gait disturbance, distal muscle weakness, scoliosis, vocal cord palsy, and respiratory muscle weakness. Peripheral neuropathy refers to damage to the nerves outside of the brain and spinal cord (peripheral nerves). Peripheral neuropathy can result from traumatic injuries, infections, metabolic problems, inherited causes and exposure to toxins. One of the most common causes of peripheral neuropathy is diabetes. Peripheral neuropathy can also be caused by autoimmune diseases, including but not limited to Sjogren's syndrome, lupus, rheumatoid arthritis, Guillain-Barre syndrome, chronic inflammatory demyelinating polyneuropathy and vasculitis.

In some embodiments, the neurodegenerative disease or disorder or a myopathy is selected from any of: CMT, ALS, spinal muscle atrophy (SMA), myasthenia gravis, Duchenne muscular dystrophy (DMD), and a neuromuscular disease or wasting disorder. In some embodiments, the neurodegenerative disease or disorder is selected from the group consisting of Parkinson's disease, Alzheimer's disease, dementia, Huntington's disease, multiple sclerosis, and cerebral ischemia.

In some embodiments, the myopathy is selected from the group consisting of Becker muscular dystrophy (BMD), Congenital muscular dystrophies (CMD), Duchenne muscular dystrophy (DMD), Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD).

As described herein, amyotrophic lateral sclerosis (ALS) is an example of a peripheral neuropathy, affecting roughly 1 in 50,000 people, that carries significant personal, societal, and economic burden. ALS manifests as degenerative changes to upper and lower motor neurons, leading to progressive muscle atrophy and weakness, increased fatigue, and problems with swallowing that typically result in respiratory failure and death. The condition is currently incurable. Riluzole is the only drug in the USA and Europe currently available for use in ALS patients but is typically only capable of prolonging life by a few months. Therapeutic regimens instead focus on a combination of physical therapy, the use of supporting braces and other adaptive equipment, and eventually wheelchairs. Consequentially, there is an urgent need to develop therapeutic options capable of ameliorating ALS symptoms or slowing disease progression to improve patient care and quality of life. It is hoped that greater mechanistic understanding of ALS etiology will permit the identification of suitable biomarkers and therapeutic targets, which will in turn lead to the development of novel treatment modalities. However, the clinical and genetic heterogeneity inherent to ALS pathophysiology makes accurate modeling of the disease difficult, and reduces the predictive power of current preclinical animal and cell-based models.

The development of high-throughput, human induced pluripotent stem cell (hiPSC)-based neuromuscular disease models can facilitate detailed analysis of disease progression and comparison of phenotypic differences across varied genetic backgrounds to isolate common features from mutation specific effects. Such models also permit evaluation of patient specific responses to different therapeutic strategies, thereby facilitating the development of more personalized treatment modalities for people suffering from neuropathic conditions like ALS).

It has been suggested that the neuromuscular junction (NMJ) is an early target for pathological onset in a number of peripheral nerve diseases and patients with ALS commonly possess defects in the maturation of their NMJs. These defects typically precede the onset of progressive muscular degeneration to the point that impairments in pre- and post-synaptic development in lower motor neuron connectivity to muscle is characteristic of early stages of the disease. It is therefore likely that defective NMJ maturation contributes to the subsequent onset of complete denervation and associated muscle weakness. Based on these data, it is clear that the NMJ is an important site of early, selective pathology in ALS. In order to prevent the degradation of synaptic structures, and improve patient mobility, it is vital to understand the upstream effects that lead to NMJ breakdown. From research conducted on familial ALS cases and animal models, a number of possible pathogenic mechanisms underlying motor neuron degeneration have emerged, including oxidative stress, mitochondrial dysfunction, misfolded protein toxicity/autophagy defects, RNA toxicity, excitotoxicity, and defective axonal transport. To date, more than 25 genes have been identified with potential pathological links to ALS, however, those most commonly observed in familial cases (and therefore the most well studied) are C9orf72 (40% of familial ALS cases), SOD1 (12-20% of familial ALS cases), FUS (roughly 5% of familial ALS cases), and TARDBP (roughly 4% of familial ALS cases). Given the wide range of downstream effects caused by mutations in these genes, identifying a root cause of ALS has remained elusive. Recent advances in hiPSC technology now makes the study and phenotypic comparison of individual patient mutations a viable possibility. However, to evaluate the effect of different ALS mutations on NMJ development requires a suitable high-throughput screening platform with which to evaluate synaptic function and breakdown.

In vitro models of NMJ physiology and function are currently available. They typically focus on dual patch analysis or assessment of muscle twitch responses to neuronal activation. However, such systems are extremely low throughput and time consuming, preventing side by side comparison of multiple disease mutations as well as simultaneous analysis of multiple dose responses to novel therapeutics. The development of a multiplexed, high-throughput NMJ functional screening platform permits comparative analysis of synaptic cross-talk across a range of ALS relevant mutations to investigate whether these pathological phenotypes result in significant differences in NMJ function and degradation within controlled in vitro environments. To fill this critical need, described herein is a structurally organized compartmentalized model of innervated human skeletal muscle and its use to study the phenotypic variance in synaptic function across ALS-relevant mutations. Human iPSC-derived motor neurons from multiple ALS patients are integrated with the structured model to investigate whether mutations with varying clinical severities exhibit notable differences in terms of NMJ development. This system demonstrates the feasibility of generating patient specific disease models for subsequent therapy evaluation and mechanistic studies. Chemical modulators of NMJ function are investigated for their capacity to alter synaptic cross-talk and demonstrate the ability to assay compound efficacy within this preclinical model. Furthermore, this system can be used to evaluate the ability for compounds undergoing clinical trial in ALS patients to ameliorate in vitro disease phenotypes within the compartmentalized hiPSC model as a proof of concept for the use of this platform in evaluation of therapeutic efficacy.

### VI. Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, “cell growth area” refers to the area on a surface substrate of the device, optionally contained within a walled chamber, wherein cells can attach, grow, and mature. It is not necessary for the cells to always be growing for the area to be termed a cell growth area. Specifically, cell growth area refers to the area in which cell bodies, comprising nuclei, are found. In some embodiments, “cell growth area” can refer to a first cell growth area, a second cell growth area, or at least one additional cell growth area.

As used herein “axon outgrowth area” also referred to as a “cell extension area” refers to the space in between the first cell growth area and the second cell growth area, e.g., in which cells can extend cellular extensions, e.g., axons, dendrites (see e.g., FIG. 1F, FIG. 3, FIG. 5D). In some embodiments, “axon outgrowth area” refers to the spatial gap or intermediate area between the area where the neurons are found and the area where the muscle cells are found. The term “axon outgrowth area” can, in the context of the area between the first and second growth areas, be used interchangeably with the terms “space”, “cell extension area”, “intermediate area”, “spatial gap” and the like. In some embodiments, a second axon outgrowth area can be found in between an additional cell growth area and the second cell growth area.

As used herein, “electrical connectivity” or “electrical communication” refers to at least two cells that are connected such that an electrical signal can be communicated between the cell types, e.g., through neurotransmitters. For example, electrical connectivity can comprise a neuromuscular junction between a neuronal cell and a muscle cell. As another example, electrical connectivity can comprise a synapse between two neuronal cells.

As used herein the terms “spatially distinct” or “spatially separated” refer to areas and cell bodies therein that do not physically contact each other. For example, the first cell growth area and the cell bodies therein are spatially separated from the second cell growth area and the cell bodies therein. In this case, the cells in the first cell growth area are electrically connected to cells in the second cell growth area (e.g., through cell extensions in the axon outgrowth area), but the cell bodies in the first cell growth are spatially separated from the cell bodies in the second cell growth area.

In some embodiments, cells are connected through synapses, i.e., synaptic connectivity. Although the cells do not physically or directly contact each other by virtue of the synaptic cleft in between the pre-synaptic cell and the post-synaptic cell, the cells are still said to be contacting each other through the synapse, or connected to each other through the synapse.

As used herein, “electroconductive cell” refers to a cell being able to conduct, generate, and/or respond to an electrical signal. It should be noted that at some level, any cell can conduct electricity. However, relevant electroconductive cells have the normal physiological function or design to generate, propagate, or respond to an electrical stimulus. Non-limiting examples of electroconductive cells can include neurons and myocytes (muscle cells). Electroconductive cells can include both naturally-occurring electroconductive cells (e.g., a muscle cell or neuron) or cells that have been engineered, e.g., genetically modified or transfected to exhibit electroconductive activity. By way of non-limiting example, a cell engineered to express at least one voltage-gated ion channel can be an engineered electroconductive cell. One of skill in the art is familiar with methods for engineering cells, which can include, but are not limited to, genetic modification, homologous recombination, transient expression, and protein transfection and can be accomplished with one or more various vectors, e.g., plasmids, naked DNA, or viral vectors.

As used herein, the term “substrate” refers to any suitable carrier material to which the cells are able to attach or adhere in order to survive and/or proliferate. In accordance with some embodiments, the cell culturing layer can be applied to the substrate to facilitate cell culturing on the cell culturing surface of the cell culturing layer.

As used herein, the term “suitable for culture”, as used in reference to a substrate for the culture of cells refers to having the necessary characteristics to allow the culture and/or maintenance of cells, e.g., for at least continuing the viability of a cell or a population of cells. A maintained population of cells will have at least a subpopulation of metabolically active cells. By way of non-limiting example, a substrate suitable for the culture of cells will not comprise any agents that are toxic to the cells; will be sterile, substantially sterile, or amenable to sterilization; and/or will be provided with a source of cell culture medium or means of containing cell culture medium in contact with the cells, etc.

The term “substantially” as used herein means for the most part, essentially the same as the character it is substantially a feature of In some embodiments, for example, a feature which is “substantially parallel” refers to features which are at least about 60%, or preferably at least about 70% or at least about 80%, or at least about 90%, at least about 95%, at least about 97% or at least about 99% or more, or any integer between 70% and 100% similar to a parallel structure. In some embodiments, for example, a surface which is “substantially smooth” is a surface which is at least about 60%, or preferably at least about 70% or at least about 80%, or at least about 90%, at least about 95%, at least about 97% or at least about 99% or more, or any integer between 70% and 100% similar to a smooth structure.

The term “nanotextured” as used herein refers to a repeating pattern of substantially parallel grooves and ridges where the heights and depths and width of the grooves and ridges are all of sub-micron scale. As such, the term nanotextured can be used interchangeably with the term “nanopatterned”.

The term “anisotropic” refers to items, such as cells, being spatially organized or arranged in a direction-related manner.

As used herein, the term “grooves” refers to a relative recess in a surface having a width, length, and depth wherein the length is greater than the width. In some embodiments, a groove can be substantially linear. In some embodiments, a groove can be linear. In some embodiments, a portion of a groove can be substantially linear. In some embodiments, a portion of a groove can be linear. In some embodiments, a groove can be recessed relative to the surface of the substrate, e.g., the surface prior to introduction of grooves and/or ridges. In some embodiments, a groove can be recessed relative to a ridge.

As used herein, the term “ridges” refers to a portion of a surface which is relatively elevated and/or raised, having a width, length, and height wherein the length is greater than the width. In some embodiments, a ridge can be substantially linear. In some embodiments, a ridge can be linear. In some embodiments, a portion of a ridge can be substantially linear. In some embodiments, a portion of a ridge can be linear. In some embodiments, a ridge can be raised and/or elevated relative to the surface of the substrate, e.g., the surface prior to introduction of grooves and/or ridges. In some embodiments, a ridge can be raised and/or elevated relative to a groove.

As used herein, the term “array” refers to an order, arrangement or series of particular elements. For example, any array of grooves and ridges refers to an arrangement of multiple grooves and/or ridges while an electrode array refers to an arrangement of multiple electrodes (e.g., 2 or more electrodes, 3 or more electrodes, or 4 or more electrodes). In some embodiments, an array can refer to an array of modules on a substrate.

As used herein, the term “cell culture chamber” refers to a reservoir in or on a substrate that can retain culture medium to support the growth and/or maintenance of cells. In some embodiments, a cell culture chamber can be a well, a depression, or an area bounded by walls. In some embodiments, at least one surface of a cell culture chamber can comprise grooves and ridges. In some embodiments, a cell culture chamber can comprise a electrode and/or an array of electrodes.

As used herein, the term “biocompatible” as used herein within the context of a substrate denotes a composition that is not biologically harmful, e.g., a material that is suitable for implantation into a subject or suitable for the growth and/or maintenance of cells in vitro. A biocompatible substrate does not cause toxic or injurious effects once implanted in the subject or when in contact with cells.

The term “biodegradable” as used herein within the context of a substrate denotes a composition that is not biologically harmful and can be chemically degraded or decomposed by natural effectors (e.g., weather, soil bacteria, plants, animals).

The term “bioresorbable” as used herein within the context of a substrate refers to the ability of a material to be reabsorbed over time in the body (e.g., in vivo) so that its original presence is no longer detected once it has been reabsorbed.

The term “bioreplaceable” as used herein within the context of a substrate as used herein, and when used in the context of an implant, refers to a process where de novo growth of the endogenous tissue replaces the implant material. A bioreplacable material as disclosed herein does not provoke an immune or inflammatory response from the subject and does not induce fibrosis. A bioreplaceable material is distinguished from bioresorbable material in that bioresorbable material is not replaced by de novo growth by endogenous tissue.

As used herein, the term “microelectrode” or “electrode” refers to a metalized layer that can receive electrical signals from and apply electrical signals to cells and tissue through direct contact or through a dielectric material.

As used herein, the term “lead” refers to a conductive element such as wire or a film that connects an electrode to a pad or signal processing or generating circuitry.

As used herein, the term “conditioning blocks” refers to electrical components that can be used to condition the signals received from cells, such as signal amplifiers and filters.

As used herein, the term “digitally sampled” refers to measuring an amplitude of a signal at a predefined time or periodically over a period of time.

As used herein, the term “computer” refers to a general purpose computing device having one or more processors and associated memories for storing programs (e.g., sequences of instructions) and data (e.g., digital representations of signals).

As used herein, the term “electro-stimulate” is used interchangeably with “stimulate” refers to contacting a cell with, or providing to a cell, a compound, signal, condition, and/or force that elicits a reaction from the cell. In some embodiments, stimulation can encompass providing an electrical impulse to a cell.

As used herein, the term “record” or “acquire” in reference to data (e.g., biopotential data) refers to storing data representative of biopotentials and/or impedances in a digital format in volatile or non-volatile memory.

An “electrode” is a structure having a high electrical conductivity, that is, an electrical conductivity much higher than the electrical conductivity of the surrounding materials, which in the present invention are typically nonconductive.

A “ground electrode” is the complementary structure used to complete the electrical circuit during extracellular recording.

An “impedance electrode”, “impedance monitoring electrode”, “impedance measurement electrode” or “impedance electrode structure” is a structure, such as an electrode, used for or that permits impedance monitoring. An “impedance electrode” is such a structure used to detect electrical signal corresponding to extracellular field potential of the cell or cell population.

As used herein, an “electrode structure” refers to a single electrode, particularly one with a complex structure (as, for example, a spiral electrode structure), or a collection of at least two electrode elements that are electrically connected together. All the electrode elements within an “electrode structure” are electrically connected.

As used herein, “electrode element” refers to a single structural feature of an electrode structure, such as, for example, a fingerlike or branched projection of an interdigitated electrode structure. An electrode structure may have a plurality of electrode elements.

As used herein, a “unitary electrode structure” refers to a single electrode that is unbranched. That is, a “unitary electrode structure” does not include a plurality of electrode elements. For example, an unitary electrode structure may be of a circle, a square or other geometry. Field potential electrodes and ground electrodes are examples of unitary electrode structures.

As used herein, a “pair of electrodes” or “electrode pair” is two or more electrode structures that are constructed to have dimensions and spacing such that they can, when connected to a signal source, operate as a unit to generate an electrical field in the region of spaces around the electrode structures. Preferred electrode structure units of the present invention can measure impedance changes due to cell attachment to an electrode surface. Non-limiting examples of electrode structure units are interdigitated electrode structure units and concentric electrode structure units. Impedance electrodes can be configured as impedance electrode pairs.

As used herein “electrode bus” is a portion of an electrode that connects individual electrode elements or substructures. An electrode bus provides a common conduction path from individual electrode elements or individual electrode substructures to another electrical connection. In the devices of the present invention, an electrode bus can contact each electrode element of an electrode structure and provide an electrical connection path to electrical traces that lead to a connection pad.

As used herein “electrode traces” or “electrically conductive traces” or “electrical traces”, are electrically conductive paths that extend from electrodes or electrode elements or electrode structures toward one end or boundaiy of a device or apparatus for connecting the electrodes or electrode elements or electrode structures to an electro-stimulation power source, an analyzer or amplifier, such as an impedance analyzer or amplifier, a voltage amplifier and the like. Electrical comniunication of electro-stimulation electrodes, impedance electrodes or extracellular recording electrodes typically involves connection to a connection pad using an “electrode trace.”

As used herein “connection pad” is an area on an apparatus or a device of the present invention which is electrically connected to at least one electrode or all electrode elements within at least one electrode structure on an apparatus or a device and which can be operatively connected to external electrical circuits (e.g., an impedance measurement circuit or a signal source or an extracellular voltage signal amplifier). The electrical connection between a connection pad and an impedance measurement circuit, an extracellular recording circuit or a signal source can he direct or indirect, through any appropriate electrical conduction means such as leads or wires. Such electrical conduction means may also go through electrode or electrical conduction paths located on other regions of the apparatus or device.

As used herein “interdigitated” means having projections coming one direction that interlace with projections coming from a different direction in the manner of the fingers of folded hands (with the caveat that interdigitated electrode elements preferably do not contact one another).

As used herein, “the . . . electrodes (or electrode structures) have substantially the same surface area” means that the surface areas of the electrodes referred to are not substantially different from each other, so that the impedance change due to cell attachment or growth on any one of the electrodes (or electrode structures) referred to will contribute to the overall detectable change in impedance to a same or similar degree as the impedance change due to cell attachment or growth on any other of the electrodes (or electrode structures) referred to. In other words, where electrodes (or electrode structures) have substantially the same surface area, any one of the electrodes can contribute to overall change in impedance upon cell attachment or growth on the electrode. In most cases, the ratio of surface area between the largest electrode and the smallest electrode that have “substantially the same surface area” is less than 10. Preferably, the ratio of surface area between the largest electrode and the smallest electrode of an electrode array is less than 5, 4. 3, 2, 1.5, 1.2 or 1.1. More preferably, the at least two electrodes of an electrode structure have nearly identical or identical surface area.

As used herein, “the device has a surface suitable for cell attachment or growth” means that the electrode and/or non-electrode area of the apparatus has appropriate physical, chemical or biological properties such that cells of interest can viably attach on the surface and new cells can continue to attach, while the cell culture grows, on the surface of the apparatus. However, it is not necessary that the device, or the surface thereof, contain substances necessary for cell viability or growth. These necessary substances, e.g., nutrients or growth factors, can be supplied. in a medium. Preferably, when a suspension of viable cardiomyocytes, neuron cells, muscle cells or other excitable cells or other adherent cells such as epithelial cells or endothelial cells is added to the “surface suitable for cell attachment” when at least 50% of the cells are adhering to the surface within twelve hours. More preferably, a surface that is suitable for cefi attachment has surface properties so that at least 70% of the cells are adhering to the surface within twelve hours of plating (i.e., adding cells to the chamber or well that comprises the said device). Even more preferably, the surface properties of a surface that is suitable for cell attachment results in at least 90% of the cells adhering to the. surface within twelve hours of plating. Most preferably, the surface properties of a surface that is suitable for cell attachment results in at least 90% of the cells adhering to the surface within eight, six, four, two hours of plating.

As used herein, “detectable change in impedance between or among said electrodes” (or “detectable change. in impedance between or among the electrode structures”) means that the impedance between or among the electrodes (or electrode structures) would have a significant change that can be detected by an impedance analyzer or impedance measurement circuit when cells attach on the electrode surfaces. The impedance change refers to the difference in impedance values when cells are attached to the electrode surface and when cells are not attached to the. electrode surface, or when the number, type, activity, adhesiveness, or morphology of cells (including, particularly, contracted vs. relaxed or non-contracted morphology, e.g., of a muscle cell or cells) attached to the electrode-comprising surface of the apparatus changes. In most cases, the change in impedance is larger than 0.1% to be detectable. Preferably, the detectable change in impedance is larger than 1%, 2%, 5%, or 8%. More preferably, the detectable change in impedance is larger than 10%. impedance between or among electrodes is typically a function of the frequency of the applied electric field for measurement. “Detectable change in impedance between or among the electrodes” does not require the impedance change at all frequencies being detectable. “Detectable change in impedance between or among said electrodes” only requires a detectable change in impedance. at any single frequency (or multiple frequencies). In addition, impedance has two components, resistance and reactance (reactance can be divided into two categories, capacitive reactance and inductive reactance). “Detectable change in impedance between or among said electrodes” requires only that either one of resistance and reactance has a detectable change at any single frequency or multiple frequencies. in the present application, impedance is the electrical or electronic impedance. The method for the measurement of such impedance is achieved by, (1) applying a voltage between or among the electrodes at a given frequency (or multiple frequencies, or having specific voltage waveform) and monitoring the electrical current through said electrodes at the frequency (or multiple frequencies, or having specific waveform), dividing the voltage amplitude value by the. current amplitude value to derive the. impedance value; (2) applying an electric current of a single frequency component (or multiple frequencies or having specific current wave form) through said electrodes and monitoring the voltage resulted between or among said electrodes at the frequency (or multiple frequencies, or having specific waveform), dividing the voltage amplitude value by the current amplitude value to derive the impedance value; (3) other methods that can measure or determine electric impedance. Note that in the description above of “dividing the voltage amplitude value by the current amplitude value to derive the impedance value”, the “division” is done for the values of current amplitude and voltage amplitude at same frequencies. Measurement of such electric impedance is an electronic or electrical process that does not involve the use of any reagents.

As used herein, “arranged in a row-column configuration” means that, in terms of electric connection, the position of a field potential electrode, or an impedance electrode or impedance electrode pair is identified by both a row position number and a column position number.

As used herein “Cell Index” or “CI” is a parameter that can derived from measured impedance values and that can be used to reflect the change in impedance values. There are a number of methods to derive or calculate Cell Index. The details of the method for calculating Cell Index, Normalized Cell Index, Delta Cell Index and cell change index can be found in U.S. patent application Ser. No. 10/705,447, filed on Nov. 10, 2003; U.S. patent application Ser. No. 10/705,615, filed on Nov. 10, 2003; U.S. patent application Ser. No. 10/987,73, filed on Nov. 12, 2004; U.S. patent application Ser. No. 11/055,639, filed on Feb. 9, 2005; U.S. patent application Ser. No. 11/198,831, filed on Aug. 4, 2005; U.S. patent application Ser. No. 11/197,994, filed on Aug. 4, 2005; U.S. patent application Ser. No. 11/235,938, filed on Sep. 27, 2005, each of which are incorporated here by reference in their entireties.

A “Normalized Cell Index” at a given time point is calculated by dividing the Cell Index at the time point by the Cell Index at a reference time point. Thus, the Normalized Cell Index is 1 at the reference time point. Generally, for an assay involving treatment of the cells with compounds or with other bio-manipulation of the cells, the reference time point is the last time point for impedance measurement before the treatment of the cells.

A “delta cell index” at a given time point is calculated by subtracting the cell index at a standard time point from the cell index at the given time point. Thus, the delta cell index is the absolute change in the cell index from an initial time (the standard time point) to the measurement time.

A “Cell Change Index” or “CCI” is a parameter derived from Cell Index and “CCI” at a time point is equal to the 1st order derive of the Cell Index with respect to time, divided by the Cell Index at the time point. Calculation of CCI is disclosed in US application 2014/0203818.

As used herein “extracellular recording” refers to measuring, monitoring and/or recording of electric potential difference between two electrodes typically caused by ionic movement or ionic current through the media or solution due to charge fluctuations across ion channels in a cell or in a group of cells. The cells are in the media or the solution. In contrast to intracellular recording where the recording electrodes are placed inside a cell through the cell membrane, the extracellular recording electrodes are located outside of the cells.

As used herein, the term “interface electronics” refers to electrical components that convert electrical signals between formats or connect electronic components to permit signals to pass between them.

As used herein, the term “signal generator” refers to an electrical component that produces an electrical signal. A signal generator can be a group of components configured to produce a signal having one or more predefined characteristics (e.g., frequency and amplitude). A signal generator can include a memory device that plays back a recorded signal. A cell or tissue sample can also be used as a signal generator.

As used herein, the term “sine wave” refers to a signal that approximates a sinusoid or sine function and provides a generally periodic signal.

As used herein, the term “signal” refers to a detected electrical potential such as a biopotential or a detected impedance. The term “signal” also refers to an electrical potential that is applied to a cell or tissue to measure impedance or elicit a response.

As used herein, the term “signal recorder” refers to a device that records signals for later use or analysis.

The term “biopotentials” as used herein refers to a voltage produced by the cells cultured on the MEA substrate, particularly muscle cells or neuronal cells on the MEA substrate. Non-limiting examples of functional parameters of biopotentials can include action potential duration (ADP), wave propagation, action potential frequency, beat frequency, action potential transmission, Vmax of the action potential, contraction force, peal to peak amplitude, end diastole to peak diastole rate and the like.

As used herein, a “field potential” refers to an electrophysiological signal which is primarily the electrical current flowing through a volume of tissue. In this situation, “potential” refers to electrical potential, or voltage, in the volume of tissue. A field potential can be, e.g., a local field potential or an extracellular field potential.

As used herein, “conduction velocity” refers to the speed at which an electrochemical impulse propagates across or along a tissue, e.g., along a muscle or neuronal tissue.

As used herein, “electrophysical profile” refers to the profile (e.g., pattern, frequency, magnitude, etc.) of electrical properties of a cell and/or tissue. Electrophysical profiles can include, by way of non-limiting example, voltage change profiles, electrical current profiles, action potential activity, field potentials, changes in ion concentrations or changes in biomolecules which regulate or are regulated by electrical activity in electroconductive cells.

As used herein, “reentrant wave patterns” refers to the pattern of an electrical impulse in a heart or heart tissue which travels in a small circle within the tissue instead of transiting the tissue and then stopping. These phenomena can cause arrhythmia.

As used herein, “impedance,” when used in reference to a property of a cell and/or tissue refers to a measure of the opposition to an electrical current in that cell or tissue.

As used herein “QT interval” refers to the time between the start of the Q wave and the end of the T wave in the heart's electrical cycle. As used herein, the term “electroconductive” such as in reference to a cell refers to the property of being able to conduct, generate, and/or respond to an electrical signal. Examples of electroconductive cells are neurons and muscle cells.

As used herein, the term “neuronal cell” or “neuron” refers to cells found in the nervous system that are specialized to receive, process, and transmit information as nerve signals. Neurons can include a central cell body or soma, and two types of projections (also called extensions): dendrites, by which, in general, the majority of neuronal signals are conveyed to the cell body; and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the nervous system.

The term “myocytes” refer to muscle cells. Sub-categories of myocytes include, for example, skeletal myocytes, smooth muscle myocytes, cardiomyocytes, as well as ESC- and iPSC-derived myocytes.

The term “cardiomyocyte” as used herein broadly refers to a muscle cell of the heart (e.g., a cardiac muscle cell). The term cardiomyocyte includes smooth muscle cells of the heart, as well as cardiac muscle cells, which also include striated muscle cells, as well as spontaneous beating muscle cells of the heart. A cardiomyocyte will generally express on its cell surface and/or in the cytoplasm one or more cardiac-specific marker. Suitable cardiomyocyte-specific markers include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor.

As used herein, the term “skeletal muscle myocyte” refers to muscle cells found in skeletal muscles, e.g., voluntary muscles anchored to bone and used to effect locomotion and maintain posture.

As used herein, “smooth muscle myocyte” refers to muscle cells found within the walls of organs and structures (e.g., the esophagus, stomach, intestines, bronchi, uterus, urethra, bladder, and blood vessels) and which is not under voluntary control.

The term “myogenically committed” or “myogenic committed” refers to a cell, such as a progenitor cell, such as an ESC or iPSC, that becomes committed to the myogenic lineage (e.g., a myogenic progenitor cell). In some embodiments, the myogenically committed cell that can continue to differentiate into a muscle cell type (e.g., a substantially pure population of skeletal muscle myocytes; e.g., a substantially pure population of cardiomyocytes) and the cell is unable to progress to form other, non-muscle cell types.

The terms “cardiac progenitor cell” and “CPC” are used interchangeably herein to refer to a progenitor cell which is capable of proliferation and giving rise to more progenitor cells having the ability to generate a large number of mother cells that can in turn give rise to differentiated, or differentiable daughter cells which can eventually terminally differentiate primarily into cells of the heart tissue, including endothelial lineages and muscle lineages (smooth, cardiac and skeletal muscles).

The term “progenitor cell” is used herein to refers to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. As indicated above, stem cells have been found resident in virtually every tissue. Accordingly, the technology described herein appreciates that stem cell populations can be isolated from virtually any animal tissue.

As used herein, the term “adult cell” refers to a cell found throughout the body after embryonic development.

As used herein, the terms “iPS cell” and “induced pluripotent stem cell” are used interchangeably and refers to a pluripotent cell artificially derived (e.g., induced by complete or partial reversal) from a differentiated somatic cell (i.e. from a non-pluripotent cell). A pluripotent cell can differentiate to cells of all three developmental germ layers.

The term “derived from” used in the context of a cell derived from another cell means that a cell has stemmed from (e.g., changed from or was produced by) a cell which is a different cell type. In some instances, for example, a cell derived from an iPS cell refers to a cell which has differentiated from an iPS cell. Alternatively, a cell can be converted from one cell type to a different cell type by a process referred to as transdifferentiation or direct reprogramming. Alternatively, in the terms of iPS cells, a cell (e.g., an iPS cell) can be derived from a differentiated cell by a process referred to in the art as dedifferentiation or reprogramming.

The term “pluripotent” as used herein refers to a cell that can give rise to any type of cell in the body except germ line cells. The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages. Pluripotency is also evidenced by the expression of embryonic stem (ES) cell markers, although the preferred test for pluripotency is the demonstration of the capacity to differentiate into cells of all three germ layers, as detected using, for example, a nude mouse teratoma formation assay. iPS cells are pluripotent cells. Pluripotent cells undergo further differentiation into multipotent cells that are committed to give rise to cells that have a particular function. For example, multipotent cardiovascular stem cells give rise to the cells of the heart, including cardiomyocytes, as well as other cells involved in the vasculature of the heart. Cell useful for in vitro differentiation to myocytes or cardiomyocytes as disclosed herein include, for example, iPS cells as well as multipotent cardiovascular stem cells or multipotent muscle progenitor cells. A major benefit of the use of iPSC or other stem cells to generate myocytes or cardiomyocytes for the compositions and methods as disclosed herein is the ability to prepare large numbers of such cells and propagate them, e.g., from a specific human patient or subject. This is in contrast to methods, compositions that rely upon the isolation and use of adult skeletal muscle cells or adult cardiac cells.

The term “phenotype” refers to one or a number of total biological characteristics that define the cell or organism under a particular set of environmental conditions and factors, regardless of the actual genotype.

As used herein, “bioactive agents” or “bioactive materials” refer to naturally occurring biological materials, for example, extracellular matrix materials such as fibronectin, vitronectin, and laminin; cytokines; and growth factors and differentiation factors that have a biological effect on a biological cell, tissue or organ. “Bioactive agents” or “bioactive materials” also refer to artificially synthesized materials, molecules or compounds that have a biological effect on a biological cell, tissue or organ. The molecular weights of the bioactive agent can vary from very low (e.g., small molecules, 200-500 Daltons) to very high (e.g., plasmid DNA, 2,000,000 Daltons). In some embodiments, the bioactive agent is a small molecule. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD). In some embodiments, a small molecule can have a molecular weight of less than 3 kD. In some embodiments, a small molecule can have a molecular weight of less than 2 kD. In some embodiments, a small molecule can have a molecular weight of less than 1 kD. In some embodiments, a small molecule can have a molecular weight of less than 700 D.

The term “isolated” or “enriching” or “partially purified” as used herein refers, in the case of an in vitro-differentiated cell e.g., neuron, myocyte or cardiomyocyte, is separated from at least one other cell type. The term “enriching” is used synonymously with “isolating” cells, and means that the yield (fraction) of cells of one type is increased by at least 10% over the fraction of cells of that type in the starting culture or preparation.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, such as immature neurons, myocytes, or cardiomyocytes, refer to a population of cells that contain fewer than about 20%, more preferably fewer than about 15%, 10%, 8%, 7%, most preferably fewer than about 5%, 4%, 3%, 2%, 1%, or less than 1%, of cells that are immature neurons, immature myocytes, or immature cardiomyocytes or immature neuron progeny, immature myocyte progeny, or immature cardiomyocyte progeny.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “lineages” as used herein refers to a term to describe cells with a common ancestry, for example cells that are derived from the same neuronal stem cell, muscle stem cell, cardiovascular stem cell or other stem cell, or cells with a common developmental fate. By way of an example only, when referring to a cell that is of endoderm origin or is “endodermal linage,” this means the cell was derived from an endodermal cell and can differentiate along the endodermal lineage restricted pathways, such as one or more developmental lineage pathways which give rise to definitive endoderm cells, which in turn can differentiate into liver cells, thymus, pancreas, lung and intestine.

The term “contacting” or “contact” as used herein in connection with contacting a cell, either present on a substrate, or in the absence of a support, with an agent as described herein, includes subjecting the cell to a culture medium which comprises that agent. The term “modulate” is used consistently with its use in the art, e.g., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “reprogramming” as used herein refers to the transition of a differentiated cell to become a pluripotent or multipotent progenitor cell. Stated another way, the term reprogramming refers to the transition of a differentiated cell to an earlier developmental phenotype or developmental stage. A “reprogrammed cell” is a cell that has reversed or retraced all, or part of its developmental differentiation pathway to become a progenitor cell. Thus, a differentiated cell (which can only produce daughter cells of a predetermined phenotype or cell linage) or a terminally differentiated cell can be reprogrammed to an earlier developmental stage and become a progenitor cell, which can both self-renew and give rise to differentiated or undifferentiated daughter cells. The daughter cells themselves can be induced to proliferate and produce progeny that subsequently differentiate into one or more mature cell types, while also retaining one or more cells with parental developmental potential. The term reprogramming is also commonly referred to as retrodifferentiation or dedifferentiation in the art. A “reprogrammed cell” is also sometimes referred to in the art as an “induced pluripotent stem” (iPS) cell.

In the context of cell ontogeny, the term “differentiated”, or “differentiating” is a relative term. A “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an atrial precursor), and then to an end-stage differentiated cell, such as skeletal muscle cells, atrial cardiomyocytes, or smooth muscle cells, which play a characteristic role in a certain tissue type, and may or may not retain the capacity to proliferate further. The term “differentiated cell” refers to any primary cell that is not, in its native form, pluripotent as that term is defined herein. The term a “differentiated cell” also encompasses cells that are partially differentiated, such as multipotent cells, or cells that are stable non-pluripotent partially reprogrammed cells. In some embodiments, a differentiated cell is a cell that is a stable intermediate cell, such as a non-pluripotent partially reprogrammed cell. It should be noted that placing many primary cells in culture can lead to some loss of fully differentiated characteristics. However, simply culturing such primary cells, e.g., after removal or isolation from a tissue or organism does not render these cells non-differentiated cells (e.g., undifferentiated cells) or pluripotent cells. The transition of a differentiated cell (including stable non-pluripotent partially reprogrammed cell intermediates) to pluripotency requires a reprogramming stimulus beyond the stimuli that lead to partial loss of differentiated character in culture.

The term “differentiation” as referred to herein refers to the process whereby a cell moves further down the developmental pathway and begins expressing markers and phenotypic characteristics known to be associated with a cell that are more specialized and closer to becoming terminally differentiated cells. The pathway along which cells progress from a less committed cell to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a more specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, in the context of this specification, the terms “differentiation” or “differentiated” refer to cells that are more specialized in their fate or function than at one time in their development. For example, in the context of this application, a differentiated cell includes a ventricular cardiomyocyte which has differentiated from cardiovascular progenitor cell, where such cardiovascular progenitor cell can in some instances be derived from the differentiation of an ES cell, or alternatively from the differentiation of an induced pluripotent stem (iPS) cell, or in some embodiments from a human ES cell line. Thus, while such a ventricular cardiomyocyte cell is more specialized than the time in which it had the phenotype of a cardiovascular progenitor cell, it can also be less specialized as compared to when the cell existed as a mature cell from which the iPS cell was derived (e.g., prior to the reprogramming of the cell to form the iPS cell).

A cell that is “differentiated” relative to a progenitor cell has one or more phenotypic differences relative to that progenitor cell and characteristic of a more mature or specialized cell type. Phenotypic differences include, but are not limited to morphologic differences and differences in gene expression and biological activity, including not only the presence or absence of an expressed marker, but also differences in the amount of a marker and differences in the co-expression patterns of a set of markers.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “tissue” refers to a group or layer of similarly specialized cells which together perform certain special functions. The term “tissue-specific” refers to a source or defining characteristic of cells from a specific tissue.

The term “genetically modified” as used herein refers to a cell or organism in which genetic information or material has been modified by human manipulation. Modification can be effectuated by chemical, physical, viral or stress-induced or other means, including introduction exogenous nucleic acid through any standard means, such as transfection, such that the cell or organism has acquired a new characteristic, phenotype, genotype, and/or gene expression product, including but not limited to a gene marker, a gene product, and/or an mRNA, to endow the original cell or organism, at a genetic level, with a function, characteristic, or genetic element not present in non-genetically modified, non-selected counterpart cells or entities.

The term “agent” refers to any entity which is normally not present or not present at the levels being administered to a cell, tissue, organ or subject. Agent can be selected from a group comprising: chemicals; small molecules; nucleic acid sequences; nucleic acid analogues; proteins; peptides; peptidomimetics; peptide derivatives; peptide analogs; aptamers; antibodies; intrabodies; biological macromolecules; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example, peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. In some embodiments, agents can be extracts made from biological materials such as bacteria, plants, fungi, or animal cells or tissues. In some embodiments, agents can be naturally occurring or synthetic compositions or functional fragments thereof. Agents can be known to have a desired activity and/or property, or can be selected from a library of diverse compounds.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a neurodegenerative disease or disorder (e.g., peripheral neuropathy) or a myopathy.

A subject can be one who has been previously diagnosed with or identified as suffering from or having a condition in need of treatment (e.g., a neurodegenerative disease or disorder or a myopathy) or one or more complications related to such a condition, and optionally, have already undergone treatment for a neurodegenerative disease or disorder or a myopathy or the one or more complications related to a neurodegenerative disease or disorder or a myopathy. Alternatively, a subject can also be one who has not been previously diagnosed as having a neurodegenerative disease or disorder or a myopathy or one or more complications related to a neurodegenerative disease or disorder or a myopathy. For example, a subject can be one who exhibits one or more risk factors for a neurodegenerative disease or disorder or a myopathy or one or more complications related to a neurodegenerative disease or disorder or a myopathy or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., ** activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof The nucleic acid can be either single-stranded or double-stranded. A single-stranded nucleic acid can be one nucleic acid strand of a denatured double- stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having a neurodegenerative disease or disorder or a myopathy, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g., a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., a neurodegenerative disease or disorder or a myopathy. The term “treating” includes reducing or alleviating at least one adverse effect or symptom of a condition, disease or disorder associated with a neurodegenerative disease or disorder or a myopathy. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A system comprising one or more components as described and/or illustrated herein.
2. A device comprising one or more elements as described and/or illustrated herein.
3. A method comprising one or more steps as described and/or illustrated herein.
4. A non-transitory computer readable medium having computer executable instructions stored thereon that, if executed by one or more processors of a computing device, cause the computing device to perform one or more steps as described and/or illustrated herein.
5. A biomimetic platform for developing physiologically ordered tissues comprising an anisotropic, multilayered tissue culture with cues configured to promote improved levels of maturation, the platform comprises a 3D scaffold-free tissue structure including hiPSC-based motor neurons and primary human myoblasts.
6. A platform comprising a field potential electrode array and an impedance electrode array, where the filed potential electrodes are configured to monitor spontaneous activity of neurons in contact with the electrode and further configured to electrically stimulate the neurons' synaptic connections; and the impedance electrode array is plated with a multilayered skeletal muscle construct.
7. A compartmentalized platform for assessing CMT neuromuscular junction functionality, the platform comprising: an electronic chip comprising a field potential electrode and an impedance electrode; and PDMS channels supporting culture channels between the filed potential electrode and the impedance electrode.

Some embodiments of the technology described herein can be defined according to any of the following numbered paragraphs:
1. A device for monitoring the electrical communication between two different electrically excitable cell types, comprising at least one module on a substrate, each module comprising:
   a. a first cell growth area, a second cell growth area, and an axon outgrowth area flanked between the first cell growth area and the second cell growth area, and
   b. a plurality of field potential electrodes and a plurality of impedance electrodes on the surface of the substrate,
      wherein the plurality of field potential electrodes are located on the surface of the first cell growth area and the plurality of impedance electrodes are located on the surface of second cell growth area, and
      wherein the plurality of field potential electrodes and the plurality of impedance electrodes are connected to an electronic interface.
2. The device of paragraph 1, wherein the axon outgrowth area has a width of at least 100 μm between the first cell growth area and the second cell growth area.
3. The device of any one of paragraphs 1-2, wherein the axon outgrowth area is configured for axons to extend from the first cell growth area to the second cell growth area.
4. The device of any one of paragraphs 1-3, wherein the axon outgrowth area comprises a nanopattern on the substrate.
5. The device of any one of paragraphs 1-4, wherein the axon outgrowth area comprises a series of parallel microchannels with a proximal and distal end, wherein the proximal end of the microchannels interfaces with the first cell growth area, and the distal end of the microchannels interfaces with the second cell growth area.
6. The device of any one of paragraphs 1-5, wherein the microchannel width is between 1 μm and 50 μm, the interchannel width is between 1 μm and 150 μm, and the microchannel depth is between 50 nm and 5000 nm.
7. The device of any one of paragraphs 1-6, further comprising at least one barrier located between the first cell growth area and the second cell growth area, wherein the barrier is configured to separate a plurality of cell bodies of cells located on the first cell growth area from cells located on the second growth area.
8. The device of any one of paragraphs 1-7, wherein the barrier is located at the interface between the first cell growth area and the axon outgrowth area.
9. The device of any one of paragraphs 1-8, wherein the barrier is located within the axon outgrowth area, and wherein the barrier is configured to separate a plurality of cell bodies of cells located on the first cell growth area from cells located on the second growth area.
10. The device of any one of paragraphs 1-9, wherein the barrier is configured to allow axons from cells located on the surface of the first cell growth area to extend into the axon outgrowth area.
11. The device of any one of paragraphs 1-10, wherein the barrier is a non-removable or removable physical barrier.
12. The device of any one of paragraphs 1-11, wherein the barrier is the same width as the axon outgrowth area.
13. The device of any one of paragraphs 1-12, wherein the plurality of field potential electrodes (FPE) is arranged in an array.
14. The device of any one of paragraphs 1-13, wherein the plurality of impedance electrodes (IE) is arranged in an array.
15. The device of any one of paragraphs 1-14, wherein the field potential electrodes (FPE) are configured to receive an electrical signal via the electrical interface from a power source and configured to deliver an electrical stimulating signal to the surface of the first cell growth area.
16. The device of any one of paragraphs 1-15, wherein the field potential electrodes (FPE) are configured to monitor any one of: spontaneous, electrically-paced, or optically-paced activity of cells in contact with the field potential electrode.
17. The device of any one of paragraphs 1-16, wherein the field potential electrodes (FPE) are configured for one or both of:
   a. monitor any one of: spontaneous, electrically-paced, or optically-paced activity of cells in contact with the field potential electrode; and
   b. electrically stimulate cells present on the first cell growth area.
18. The device of any one of paragraphs 1-17, wherein the field potential electrodes can electrically stimulate synaptic connections of cells present on the first cell growth area with the cells present on the second growth area.
19. The device of any one of paragraphs 1-18, wherein the impedance electrodes (IE) are communicatively coupled via the electrical interface to at least one analyzing module in the form of an impedance analyzer, thereby permitting impedance monitoring from excitable cells attached to the surface of the second growth area.
20. The device of any one of paragraphs 1-19, further comprising a third cell surface area, wherein an edge of the third cell surface area interfaces with a proximal edge of the first cell growth area and the axon outgrowth area, or a distal edge of the second cell growth area and the axon outgrowth area.
21. The device of any one of paragraphs 1-20, further comprising a plurality of neuronal cells on the first cell growth area and a plurality of contractile cells or muscle cells on the second cell growth area.
22. The device of any one of paragraphs 1-21, wherein the plurality of muscle cells on the second cell growth area is a 2D monolayer or an engineered 3D skeletal muscle construct.
23. The device of any one of paragraphs 1-22, wherein the plurality of neuronal cells is derived from iPSCs obtained from a healthy subject or a subject with a neurodegenerative disease or disorder or a myopathy.
24. The device of any one of paragraphs 1-23, wherein the plurality of muscle cells is derived from iPSCs obtained from a healthy subject or a subject with a neurodegenerative disease or disorder or a myopathy.
25. The device of any one of paragraphs 1-24, wherein the plurality of neuronal cells comprise motor neurons, and the plurality of muscle cells comprise skeletal muscle cells.
26. The device of any one of paragraphs 1-25, wherein the plurality of neuronal cells comprise sympathetic neurons, and the plurality of muscle cells comprise smooth muscle cells.
27. The device of any one of paragraphs 1-26, wherein the plurality of neuronal cells comprise sympathetic neurons, and the plurality of muscle cells comprise cardiac muscle cells or cardiomyocytes.
28. The device of any one of paragraphs 1-27, further comprising an additional cell type on any one or more of: the first cell growth area, the second cell growth area or the axon outgrowth area.
29. The device of any one of paragraphs 1-28, wherein the additional cell type is selected from any of: Schwann cells, microglia, astrocytes or satellite cells.
30. The device of any one of paragraphs 1-29, wherein the additional cell type is Schwann cells, wherein the Schwann cells are located on the first cell growth area or axon outgrowth area, or both.
31. The device of any one of paragraphs 1-30, wherein the neurodegenerative disease or a myopathy is selected from any of: CMT, ALS, SMA, myasthenia gravis, DMD, and a neuromuscular disease or wasting disorder.
32. The device of any one of paragraphs 1-31, wherein the neuronal cells on the first cell growth area extend axons through the axon outgrowth area and into the second cell growth area comprising a plurality of muscle cells.
33. The device of any one of paragraphs 1-32, wherein the neuronal cells that extend axons through the axon outgrowth area make synaptic connections with muscle cells present on the second growth area.
34. The device of any of paragraphs 1-33, wherein the first cell growth area comprises a nanopatterned surface, or the second cell growth comprises a nanopatterned surface, or both the first and the second cell growth surfaces comprise a nanopatterned surface.
35. The device of any one of paragraphs 1-34, wherein the nanopattern on the first cell growth area is the same as the nanopattern on the second cell growth area.
36. The device of any one of paragraphs 1-34, wherein the nanopattern on the first cell growth area is different to the nanopattern on the second cell growth area.
37. The device of any one of paragraphs 1-36, wherein the nanopatterned surface provides anisotropic cues that promote improved levels of maturation of neuronal cell types, motor neurons and myocytes.
38. The device of any one of paragraphs 1-37, comprising an array of modules on the substrate.
39. A system for measuring the electrical conductance from one cell type to a second cell type comprising:
   a. a device according to any of paragraphs 1-38,
   b. an electronic interface for stimulation of field potential electrodes on the device, and
   c. an electronic interface that permits recording of electrical activity from the impedance electrodes.
40. A method for measuring the electrical conductance from one cell type to a second cell type comprising:
   a. providing a device of any of paragraphs 1-38, wherein the device comprises a first cell type on the first cell growth area, and a second cell type on the second cell growth area, and wherein the first cell type extends axons across the axon outgrowth area to the second cell type in the second cell growth area;
   b. providing electrical stimulation to the first cell type via the field potential electrodes, and
   c. recording electrical activity of the second cell type via the impedance electrodes.
41. The method of paragraph 40, wherein the first cell type is a plurality of neuronal cells.
42. The method of any one of paragraphs 40-41, wherein the second cell type is a plurality of contractile or muscle cells.
43. The method of any one of paragraphs 40-42, wherein the plurality of muscle cells is a monolayer or an engineered skeletal muscle construct.
44. The method of any one of paragraphs 40-43, wherein the plurality of neuronal cells is derived from iPSCs obtained from a healthy subject or a subject with a neurodegenerative disease or disorder or a myopathy.
45. The method of any one of paragraphs 40-44, wherein the plurality of muscle cells is derived from iPSCs obtained from a healthy subject or a subject with a neurodegenerative disease or disorder or a myopathy.
46. The method of any one of paragraphs 40-45, wherein the plurality of neuronal cells comprise motor neurons, and the plurality of muscle cells comprise skeletal muscle cells.
47. The method of any one of paragraphs 40-46, wherein the plurality of neuronal cells comprise sympathetic neurons, and the plurality of muscle cells comprise smooth muscle cells.
48. The method of any one of paragraphs 40-47, wherein the plurality of neuronal cells comprise sympathetic neurons, and the plurality of muscle cells comprise cardiac muscle cells or cardiomyocytes.
49. The method of any one of paragraphs 40-48, wherein the plurality of neuronal cells, or plurality of muscle cells, or both, are genetically modified cells to introduce one or more mutations for a neurodegenerative disease or myopathy.
50. The method of any one of paragraphs 40-49, wherein the plurality of neuronal cells, or plurality of muscle cells, or both, are isogenic controls of a genetically modified cell that has one or more mutations introduced for a neurodegenerative disease or myopathy.
51. The method of any of paragraphs 40-50 for use to assess electrical conductance across at least one neuromuscular junctions (NMJ) between an axon extended from the first cell type and the cell bodies of the second cell type.
52. An assay for assessing an agent for modulation of electrical signaling from one cell type to another cell type, comprising:
   a. providing a device of any one of paragraphs 1-38, wherein the device comprises a first cell type located on surface of the first cell growth area, and a second cell type located on the second cell growth area, and wherein the first cell type extends axons across the axon outgrowth area from the first cell area to the second cell type in the second cell growth area;
   b. contacting the first cell type, second cell type, or both, with an agent;
   c. providing electrical stimulation to the first cell type via the field potential electrodes;
   d. recording electrical activity of the second cell type via the impedance electrodes; and
   e. detecting a change in electrical activity of the second cell type recorded via the impedance electrodes in the presence of the agent as compared to the absence of the agent.
53. The assay of paragraph 52, wherein the first cell type is a plurality of neuronal cells.
54. The assay of any one of paragraphs 52-53, wherein the second cell type is a plurality of contractile or muscle cells.
55. The assay of any one of paragraphs 52-54, wherein the plurality of muscle cells is a monolayer or an engineered skeletal muscle construct.
56. The assay of any one of paragraphs 52-55, wherein the plurality of neuronal cells is derived from iPSCs obtained from a healthy subject or a subject with a neurodegenerative disease or disorder or a myopathy.
57. The assay of any one of paragraphs 52-56, wherein the plurality of muscle cells is derived from iPSCs obtained from a healthy subject or a subject with a neurodegenerative disease or disorder or a myopathy.
58. The assay of any one of paragraphs 52-57, wherein the plurality of neuronal cells comprise motor neurons, and the plurality of muscle cells comprise skeletal muscle cells.
59. The assay of any one of paragraphs 52-58, wherein the plurality of neuronal cells comprise sympathetic neurons, and the plurality of muscle cells comprise smooth muscle cells.
60. The assay of any one of paragraphs 52-59, wherein the plurality of neuronal cells comprise sympathetic neurons, and the plurality of muscle cells comprise cardiac muscle cells or cardiomyocytes.
61. The assay of any one of paragraphs 52-60, wherein the plurality of neuronal cells, or plurality of muscle cells, or both, are genetically modified cells to introduce one or more mutations for a neurodegenerative disease or myopathy.
62. The assay of any one of paragraphs 52-61, wherein the plurality of neuronal cells, or plurality of muscle cells, or both, are isogenic controls of a genetically modified cell that has one or more mutations introduced for a neurodegenerative disease or myopathy.
63. The assay of any of paragraphs 52-62 to assess neuromuscular junctions (NMJ) between the axons from the first cell type and the cell bodies of the second cell type.

### EXAMPLES

The devices and methods as described herein are directed to ordered muscle tissue engineering and have overlapping focuses that draw on the fields of micro/nano-technology, biomaterials, tissue engineering, stem cell biology, and neuromuscular physiology. In some embodiments, described herein is a high-throughput NMJ screening platform that can identify changes in synaptic cross-talk in response to treatment with neuromuscular agonists and antagonists (see e.g., Example 1).

In some embodiments, this system can be used to investigate whether hiPSC-derived motor neurons carrying different ALS-relevant mutations (generated via CRISPR/Cas9 gene editing) exhibit distinct structural, electrophysiological, and synaptic properties in vitro (see e.g., Example 2). Despite different underlying mutations, hiPSC-derived neurons from ALS patients can exhibit a degree of consistency in terms of structural, functional, and synaptic defects. Although primitive synaptic contacts have been demonstrated within in vitro systems previously, the devices described herein, comprising nanotopographic substrates that enhance both muscle and neuron maturation, can lead to improved synaptic development and greater stratification of ALS disease phenotypes. Without wishing to be bound by theory, it is proposed that the severity of NMJ malformation correlates with the clinical severity of the mutation investigated.

In some embodiments, ALS patient-derived hiPSC-derived motor neurons (from the Target ALS and Answer ALS biobanks) are used within the 3D NMJ model and can be used to test whether application of compounds from ALS-focused clinical trials leads to the recovery of a normal phenotype (see e.g., Example 3). These Examples, supported by pharmacological analysis in Example 1, can demonstrate the utility of the engineered platform as a suitable system in which to screen therapeutic strategies for their capacity to ameliorate the ALS NMJ phenotype and aid the successful transition of such techniques to clinical evaluation in patients.

In some embodiments, MEA devices can be used to study NMJ function in cells derived from Charcot-Marie-Tooth disease (CMT) patients (see e.g., Example 4).

### Example 1

A High-Throughput, Compartmentalized Platform for Investigating Neuromuscular Connectivity and Synaptic Function.

Previous work has demonstrated that synaptic contacts can form between cultured motor neurons and muscle cells. However, such systems require significant adaptation to provide the throughput necessary for meaningful comparison of ALS mutant phenotypes and dose response drug studies. The combination of guided topographic cell culture techniques with compartmentalized microfluidic chambers and electrode-based in situ functional assessment permits simultaneous assessment of multiplexed hiPSC nerve-muscle spatially separated cultures for screening of human NMJ function.

Previous work has shown that rodent motor neuron-muscle co-cultures form functional synapses in vitro that permit neuronal-controlled activation of muscle contraction (see e.g., Smith et al., Technology 2013, 1:37-48). While the cantilever system used to assess muscle function in Smith was reliable and reproducible, the reliance on sequential assessment of cantilever deflections prevented movement to higher throughput analysis. As such, high-throughput electrode arrays can be used for simultaneous analysis of both neuronal and skeletal muscle function. Using a 48-well multielectrode array (MEA) system, nanotopographic substrates, fabricated using an ion permeable polymer (Nafion) to permit detection of electrical signals through the substrate see e.g., FIG. 6A-6C), promoted increased spontaneous electrophysiological activity in both skeletal muscle and neuronal cultures, in concordance with structural improvements in these wells (see e.g., FIG. 6D-6E). Furthermore, skeletal muscle firing rates increased when co-cultured with innervating motor neurons (see e.g., FIG. 6F). Since neurons were cultured directly on top of muscle cells in these experiments, it was not possible to separately stimulate neuron and muscle populations, making controlled study of synaptic behavior difficult. Furthermore, this MEA platform simply makes use of field potential electrodes, meaning no assessment of muscle contractile function can be performed (e.g., with impedance electrodes). Described herein is a compartmentalized system utilizing field potential electrodes for the neuronal compartment and impedance electrodes to assay contractile function in the muscle compartment. Work with contracting hiPSC-derived cardiomyocytes demonstrates that changes in impedance can be used to provide a relative measurement of contraction (see e.g., 7A-7B), thereby permitting comparative analysis between experimental conditions.

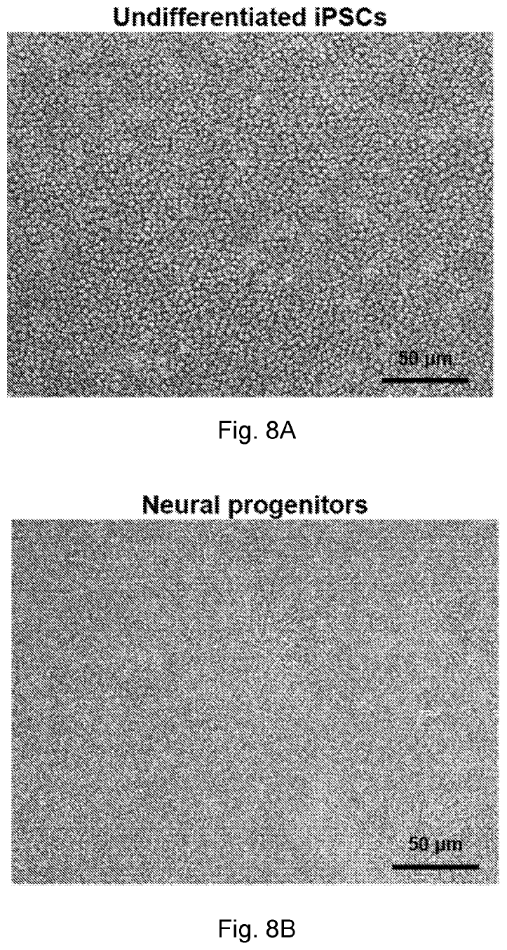

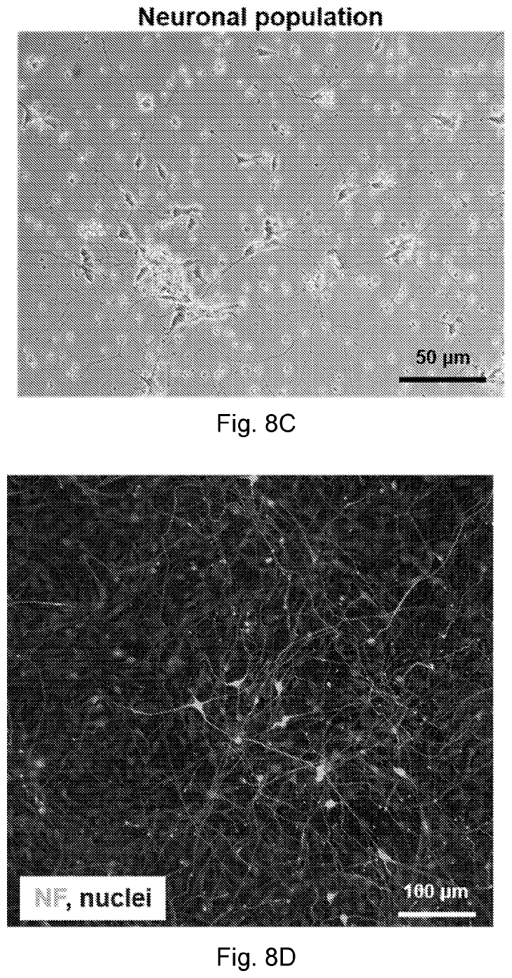

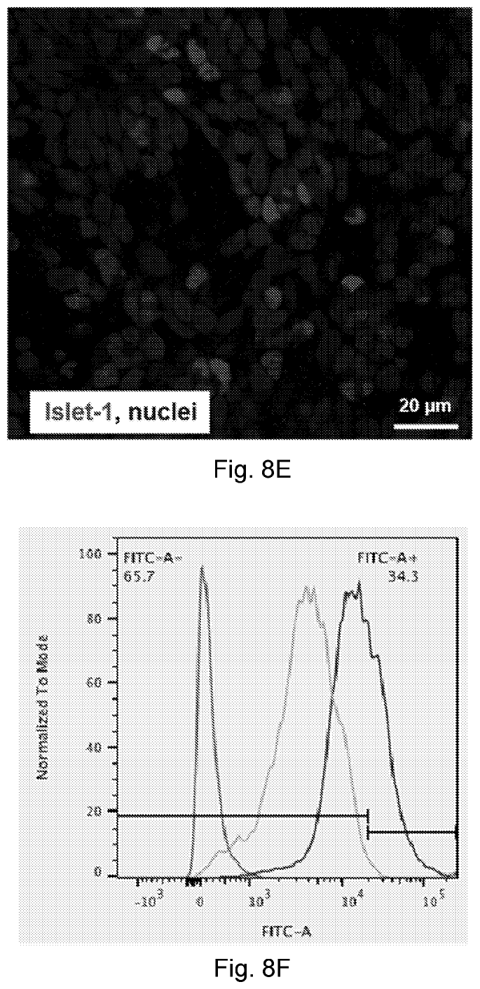

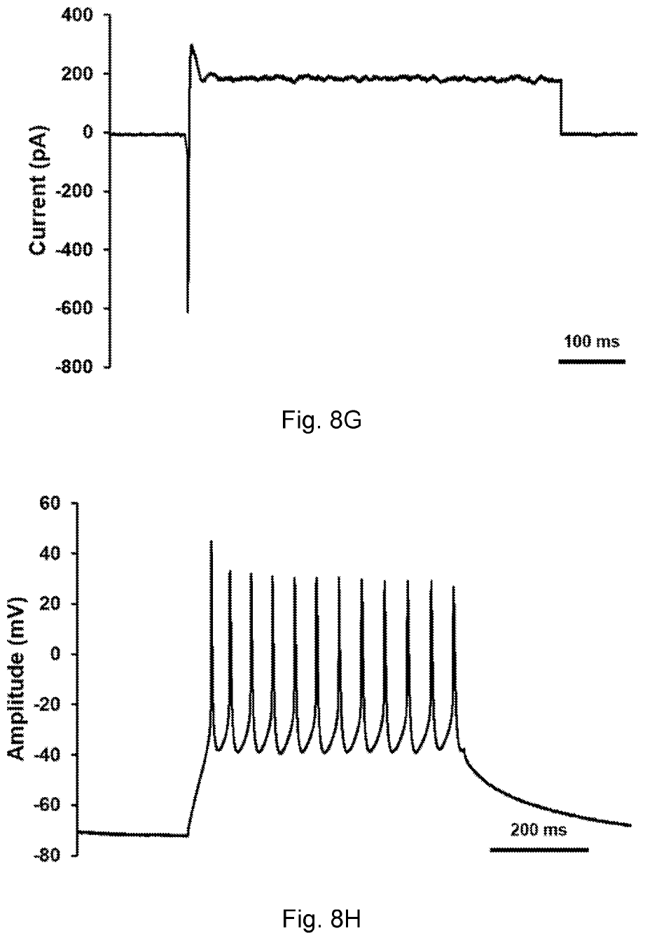

hiPSC-derived motor neurons can be produced that exhibit typical neuronal morphology and electrophysiological function and stain positive for motor neuron markers such as Islet-1 and neurofilament (see e.g., FIG. 8A-8H). Using these cells in conjunction with commercially-sourced primary human skeletal muscle cells (Lonza™), the high-throughput platform can be used to assay NMJ activity and then used to investigate whether treatment with known agonists and antagonists leads to detectable changes in synaptic function.

### Platform Design and Fabrication

To facilitate assessment of NMJ function between hiPSC-derived neurons and primary skeletal muscle cells, the assay can be established within a 48-well culture format, incorporating both field potential and impedance electrodes. Electrodes are fabricated on glass substrates using gold sputtering. Once gold deposition is complete it is insulated with a layer of SU-8, and photolithography is then used to expose electrodes that conform to the desired well design. Electrodes are positioned as detailed (see e.g., FIG. 1A, FIG. 2A-2C, FIG. 3). The design comprises a series of interdigitated impedance electrodes alongside 100 μm diameter microelectrodes forming a 4×4 grid within a 6 mm circular culture area. One electrode within the field potential electrode array is utilized for stimulation and pacing of the neuronal cultures, while the others are used for recording induced field potentials. Data collected from a prototype device illustrate the ability to make such simultaneous recordings within a multi-well format (see e.g., FIG. 7A-7B). The anisotropy of spreading resistance, implemented through a large grounding electrode closely situated to the pacing electrode, coupled with the substantial distance between pacing and recording electrodes, bias any artifact currents toward the grounding electrodes and not the recording electrodes, minimizing discharge artifacts. The substantial distance between pacing and recording electrodes also minimizes the coincidence of the initial biphasic stimulus with the recorded field potential waveform. Once electrode deposition is complete, 3D printed wells are generated and bonded to the electrode surface with a UV-curable adhesive.

Using a capillary force lithography technique, the nanopatterned substrate necessary to facilitate neuronal and skeletal muscle structural and functional maturation, as well as guide developing neurites towards the muscle compartment, is fabricated by pressing a nanogrooved PDMS master to Nafion resin and allowing the polymer to cure via solvent evaporation for 48 hours (see e.g., FIG. 4). Nafion is highly durable at physiological temperatures, and is inherently cell and protein adhesive, eliminating the need for pre-use surface treatment in cell culture experiments. The master has dimensions of 800 nm groove width, 800 nm ridge width, and 600 nm height, which has been shown to enhance skeletal muscle structure and function as well as motor neuron axon guidance and spontaneous firing rates (see e.g., FIG. 6A-6F). Patterned surfaces can be assessed for pattern conformity by scanning electron and atomic force microscopy prior to cell-based experiments.

Wells are fitted with a rubberized internal chamber made from polydimethylsiloxane (PDMS). This chamber contains a central barrier supporting microscale channels that permit neurites to pass through while preventing migration of neuron or muscle soma. PDMS chambers are fabricated from a silicon mold and plasma bonded to the patterned electrode substrates prior to cell culture. Testing with these devices demonstrates the ability to fabricate these compartmentalized chambers and the capacity for cultured neurons to extend neurites through the provided channels (see e.g., FIG. 1A-1F, FIG. 5A-5D).

The recording hardware and software necessary to use the proposed platform are configured and built exclusively to accommodate the plate design. The electrode recording hardware comprise the instrument (hardware & firmware) and user interface. Key elements of the hardware are the “interface board” and the “signal process board”. The “interface board” comprises a mating board for directly connecting the disposable 48-well electrode plate. The “signal process board” is engineered to maximize the signal-to-noise ratio, as well as amplification of the detected output from the interface board. Impedance recordings are taken at 10 kHz. Complex impedance analysis can be achieved using a digital lock-in amplifier implemented through a field-programmable gate array to permit data acquisition at suitable rates and resolutions for analyzing skeletal muscle contractility. The hardware incorporates temperature and atmospheric control systems to provide stable culture conditions during recording.

Stimulation functionality is incorporated into the hardware such that stimulation settings for each well can be dictated independently. The user interface is run by a computer and serially connected to the hardware. The software permits the end user to display multi-channel simultaneous output from the 48 wells with the capacity to focus on particular wells or groups of wells to expedite comparison across conditions. In addition, the software provides a variety of noise filters to further clean recorded signals in real-time, as well as functions for depolarizing peak identification, spontaneous firing rate, peak amplitude, and burst fire duration measurements. The software can be written in MATLAB, permitting export to analysis software, such as Excel. The specifications for the hardware are provided below in Table 1.

TABLE 1
Technical specifications for recording hardware
Recording

| | |
|---|---|
| Total voltage gain | 1200 (61 dB) |
| Input-referred noise | <3 μV RMS (200 Hz-5 kHz) |
| Low-frequency corner | 0.5 Hz |
| High-frequency corner | 10 kHz |
| Sample and hold | Simultaneous all channels |
| Sample rate | 12.5 kHz |

Temperature control

| | |
|---|---|
| Range | Ambient +5° C. to 40° C. |
| Resolution | ±0.1° C. |

Stimulation

| | |
|---|---|
| Stimulation modality | Current with compliance voltage |
| Stimulation voltage | ±1.5 V |
| Stimulation current | ±250 pA |
| Stimulus phase duration | 0.05-0.25 ms |

Differentiation of hiPSCs into Motor Neurons

Human iPSCs from at least 2 separate hiPSC lines can be compared. For example, the WTC11 line can be used. In addition, a urine-derived line (UC-2) can be used (see e.g., FIG. 8A-8H). This line was generated using a non-integrating Sendai virus to reprogram urine-derived cells to a pluripotent state. As such, they represent a means to obtain patient samples in a completely non-invasive manner and generate hiPSCs from them using a reprogramming method that makes no permanent changes to the donor genome. Human iPSC colonies can be expanded and maintained in a feeder-free cell culture medium (e.g., mTeSR™) prior to differentiation. Human iPSCs are then differentiated into regionally unspecified neural progenitor cells following the method of Shi et al. (see e.g., Shi Y et al., Nat Protoc. 2012, 7:1836). These cells can then be exposed to culture conditions promoting motor neuronal differentiation. Using these methods, motor neuron populations can be produced that are 30-40% positive for standard motor neuron markers, Islet-1, HB-9, and choline acetyltransferase (ChAT) (see e.g., FIG. 8A-8H). To further purify these cultures, magnetic activated cell sorting can be employed in combination with a primary antibody specific for p75 nerve growth factor receptor to enrich the populations for motor neurons. Such methods produce >95% pure motor neurons and these populations can be used for downstream experiments. Human iPSC neurons can be cultured until they reach day 40-45 post differentiation. Data with UC2 iPSC-derived motor neurons indicate that cells subjected to this differentiation protocol reach functional maturity by day 40 (see e.g., FIG. 8G-8H). At this point, cells can be passaged onto experimental substrates for subsequent analysis.

Differentiation of hiPSCs into Skeletal Muscle

Primary human myoblasts from Lonza™ can be used to generate the skeletal muscle cultures. These cells are plated at low density in Lonza™ growth medium. Once confluent, these cells are switched into a differentiation medium, comprising 2% horse serum and 10 ng/mL insulin-like growth factor 1 in high glucose DMEM, to induce differentiation into myotubes. Myotube cultures are maintained in this medium for a further 7 to 10 days prior to analysis. Undifferentiated myoblasts are characterized by immunocytochemistry for the muscle specific structural marker desmin. Confocal images of these stains can be used to calculate population purity. Experiments with these cells highlight at least 80% purity across multiple passages, indicating the stability of this cell population across multiple experiments. Gene expression patterns can be characterized by qRT-PCR. Expression of Pax7, Myf5, MyoD, and myogenin can be assessed to ensure the cultured myoblasts possess correct transcription profiles. Myoblasts can then be pushed into terminal differentiation to form myotubes as described above. These myotubes can be examined for the development of striated sarcomeres and contractile function to provide further evidence of the suitability of the proposed method for producing functional skeletal muscle from these primary human precursors.

### In Vitro Testing of Electrode Sensitivity and Baseline NMJ Function

Plates with electrode noise of 20 μV±5 and impedance variability of 10 mΩ±2 are sufficient for downstream use. The motor neuron monolayer is plated in the field potential compartment, and the skeletal muscle monolayer is plated in the impedance compartment, and the cells are allowed to mature (e.g., for 7, 10, 14, 18, or 21 days). Recordings collected at each time point demonstrate the ability for the optimized design to collect electrical data from cultured neurons and impedance-based contractile measurements from skeletal muscle. The time course permits simultaneous assessment of NMJ function over time and can establish optimal endpoints for further study. Neuron firing rate and depolarizing spike amplitude collected and compared across all wells demonstrate the reproducibility of electrophysiological data capture in this system. Monitoring of contraction-induced changes in impedance demonstrate the ability to compare relative contraction magnitude, velocity, and relaxation speed across wells. Both spontaneous and electrically paced activity in cultured neurons can be assessed at each time-point. Stimulation protocols involve varied pacing of biphasic 100 mV, 200 mV, 300 mV, 400 mV, or 500 mV pulses with 1 msec, 5 msec, 10 msec, 15 msec, or 20 msec durations. Optimal stimulation parameters are those with increased ability to elicit contractions in the muscle compartment; such optimized parameters can be used in drug studies. Recordings collected from all 48 wells of the system and repeated across at least 10 independent plates gauge well and plate variability.

### Analysis of NMJ Functional Sensitivity to Drug-Induced Modulation

To validate the system as a means to perform high-throughput predictive NMJ efficacy/toxicity screening, the changes in functional phenotypes in hiPSC-derived nerve-muscle spatially separated cultures exposed to rising concentrations of known neuromuscular synaptic agonists and antagonists can be investigated (see e.g., Table 2). Spatially separated cultures can be established within the platform as described above and maintained to a suitable time-point such that a plateau in spontaneous neuronal activity is reached. Each compound is then be tested in a blind fashion, over an appropriately wide dosage range (e.g., at least 5 dose-points per compound). Spatially separated cultures are first assessed for spontaneous activity and are then electrically stimulated using optimized parameters, as described above. Endpoint analysis can focus on changes in the firing rate and synchronicity of muscle contractions in response to neuronal stimulation. In addition, cell viability can be tested using a caspase-3 colorimetric assay (Abcam 39401) to determine the toxic effect of the compound on cell survival. The outcomes using the platform can then be correlated with the known adverse effects of each drug, i.e. available data on compound activity in model systems and/or any reported in vitro data collected using other assays. Consistency of responses measured using the assay can be compared across both lines and expressed relative to untreated controls.

TABLE 2
Compounds for assessment of NMJ function

| Class | Compound | Mode of action |
|---|---|---|
| Antagonists | AF64A | An active site directed irreversible inhibitor of choline |
| | | acetyltransferase. Prevents synthesis of Ach and therefore |
| | | reduces transmitter release. |
| | Vesamicol | Blocks transport of ACh into presynaptic vesicles, thereby |
| | | inhibiting synaptic transmission. |
| | Botulinum toxin | Targets SNARE proteins to block transmitter release. |
| | ω-conotoxin/ | Block P/Q Ca²⁺ channels in nerve terminals to prevent |
| | ω-agatoxin | neurotransmitter release. |
| Agonists | 4-aminopyridine | Transmitter release enhancer that blocks presynaptic K⁺ |
| | | channels to prolong presynaptic action potentials. |
| | α-latrotoxin | Produces uncontrolled discharge of all synaptic vesicles in |
| | | motor nerve terminals. |
| | Carbachol/ | Maintain open state of ligand-gated channels, including |
| | decamethonium | AChRs. |
| | Neostigmine/ | Inhibit AChE, thereby preventing ACh inactivation. |
| | edrophonium/ | |
| | prostigmine | |

Experimental groups consist of untreated, carrier control treated, and then 5 to 7 doses of each compound to be tested. All analyses are blinded by a third party to ensure the achievement of robust and unbiased results. Significant differences between groups can be evaluated using appropriate statistical tests (ANOVA with post-hoc Tukey's tests for multiple comparisons) performed on SigmaPlot statistics software. In all experiments, a p value of less than 0.05 is considered significant. In addition, control tests are run with just neurons (no muscle cells in impedance compartment) or just muscle (no neurons in field potential compartment) to ensure the observed result is only achieved when both cell types are present.

The described platform permits direct and simultaneous analysis of NMJ function across 48 wells. Connectivity between the 2 compartments, measured by the degree of synchronous activity in response to neuronal stimulation, improves with time as both cell types mature on the patterned surfaces (see e.g., FIG. 6A-6F). Treatment of NMJs with agonist compounds increases spontaneous and electrically evoked activity in the spatially separated cultures and observed responses are dose dependent. Conversely, a dose dependent reduction in NMJ activity occurs when treated with antagonist compounds.

The required cell types can be generated with minimal difficulty. The 40-45 day time point was determined based on generating motor neurons with the UC2 line. WTC11 line may take longer or shorter to reach functional maturity and the longevity of motor neurons produced from different lines may make comparisons at a single time point more difficult. Functional assessment (by patch clamp and MEA analysis) of control neurons from both lines can be conducted at days 35, 40, 45, 50, 55, and 60. From these time points, an endpoint for which comparable levels of function are achieved across both genetic backgrounds can be identified. If cell death in a certain line occurs before others have reached maturity, it can make a single time point unrealistic. In this case, different endpoints can be adopted for each line based on when those cells reach functional maturity. As an indication of mature neurons, the following values can be used: a resting membrane potential of below −40 mV, repetitive firing observed in at least 50% of cells patched, and a mean rate of burst fire on MEAs greater than 0.5 Hz. In the case that electrical stimulation creates artifacts that prevent analysis of evoked neuronal firing rate, the software can be updated to switch off recording during the very brief period of electrical stimulation to ensure subsequent measurements from neurons can be made.

### Example 2

Investigation of the Functional Effect of Incorporating Neurons Carrying ALS-Relevant Mutations Into the Compartmentalized Assay System

Functional differences can be observed in the electrophysiology of hiPSC-derived motor neurons carrying ALS patient mutations, but to what extent these changes correlate with functional defects in synaptic development at the NMJ remains to be seen. Without wishing to be bound by theory, it is proposed that functional deficits in motor neurons expressing ALS mutations leads to impaired synaptic development within the in vitro platform, and it is proposed that despite genetic differences, lines bearing different mutations display similar synaptic defects in this model, indicating a common symptomatic progression regardless of specific genetic perturbation.

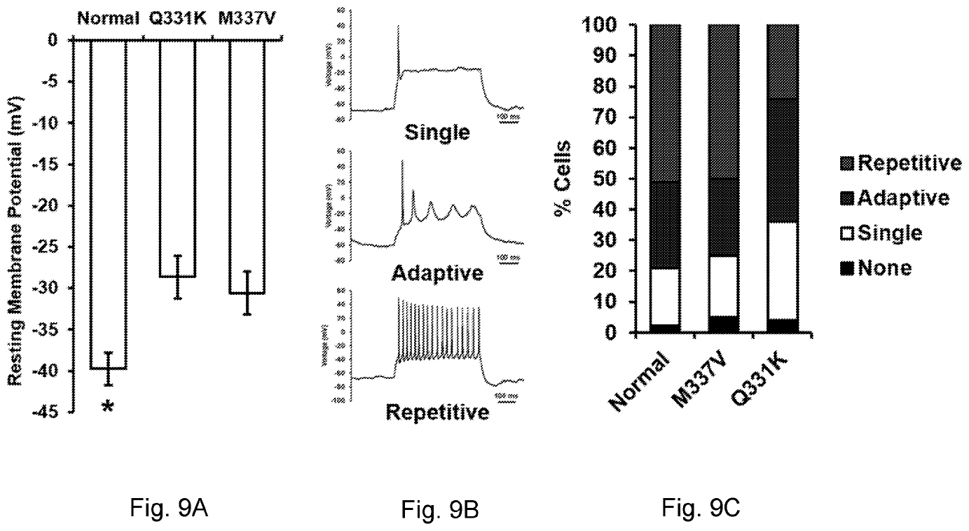

Whole cell patch clamp analysis was performed on hiPSC-derived motor neurons with CRISPR/cas9 generated ALS-associated mutations including TDP-43 Q331K and TDP-43 M337V, as well as the wild-type parental lines. Analysis revealed that motor neurons bearing either of the two TDP-43 mutations exhibited less negative resting membrane potentials than controls, and neurons with the Q331K mutation additionally displayed a reduced capacity to fire repetitive trains of action potentials following current injection (see e.g., FIG. 9A-9C). This observation confirms a similar finding reported for motor neurons differentiated from ALS patient-derived pluripotent stem cells bearing a TDP-43 mutation (see e.g., Devlin et al. Nat Commun. 2015, 6:5999). The fact that motor neurons supporting ALS mutations display distinct in vitro functional phenotypes lends credence to the notion that their capacity to form synapses can also be impaired.

hiPSC-derived motor neurons and muscle cells can be used for assessing synaptic function in ALS mutants. Using the platform as described herein (see e.g., Example 1), different mutants are investigated to determine whether different mutants display distinct functional profiles and to correlate any observed functional defects with an array of molecular and structural assays to provide comprehensive comparisons of the degree of NMJ malformation across different patient mutations.

### Production of ALS Mutant Lines

For all studies involving gene editing, the effects of ALS mutations in the two iPSC lines described in Example 1 are compared to account for line specific responses to the mutation. Experiments are performed with three separate mutant clones to account for possible effects of off-target mutations. Mutations are generated in 3 genes associated with familial ALS: TDP-43 point mutations M337V (mild phenotype) and Q331K (severe phenotype), as well as SOD1 G93A and C9orf72. ALS mutations are engineered using the CRISPR-Cas9 gene editing system. Mutations are generated as described by Miyaoka et al. (Miyaoka et al. Nat Methods. 2014, 11:291-3, which is incorporated herein by reference in its entirety). In each case, the targeted genes are sequenced prior to subsequent experimentation to confirm that the mutation has been correctly accomplished.

### Analysis of NMJ Function in ALS Lines

Once the hiPSC lines are established, their capacity to produce motor neurons can be assessed using analysis methods as described herein (see e.g., Example 1). Validated lines are then differentiated into motor neurons and cultured (e.g., spatially separated) with muscle cells derived from isogenic control hiPSCs. Functional metrics as described herein (see e.g., Example 1) are be evaluated at multiple time points over a 3-week time course to ascertain any changes in functional performance with time. All collected data are compared to the same metrics recorded from healthy isogenic control neurons.

### Assessment of NMJ Development in ALS Mutant Spatially Separated Cultures

In addition to functional assessment, a comprehensive assessment of NMJ development at endpoint in healthy versus diseased spatially separated cultures can be conducted using a panel of metrics described in Table 3. Synapse morphology is assessed using immunocytochemistry. A functional assay is fabricated on glass substrates, amenable to high resolution confocal microscopy for this purpose. The compartmentalized nature of the platform permits isolation of neurons and muscle cells for independent transcriptomic analysis. qRT-PCR can be utilized to assess cultures for markers of NMJ development as well as skeletal muscle maturation in order to see whether spatially separated culture enhances muscle development and whether this effect is blunted in culture with mutant neurons. Lastly, a subset of the compounds tested in Example 1 (see e.g., Table 2) with the strongest effect on synapse activity are utilized to test whether response to chemical stimulation or inhibition of the NMJ is altered in mutant neuron spatially separated cultures.

TABLE 3
Metrics for assessment of NMJ generation, maturation, and stability within the in vitro platform

| Parameter | Details | Technique |
|---|---|---|
| Synapse | Number of single innervated myotubes vs. poly- | Immunocytochemistry |
| morphology | innervated myotubes | |
| | AChR cluster diameter | |
| | Degree of invaginations - movement towards | |
| | “pretzel-like” morphology | |
| | Number of AChR clusters per myotube | |
| | Number of myotubes contacting innervating motor | |
| | neuron terminal vs. number without neuronal contact | |
| Transcriptome | LRP4, Dok7, MUSK, Agrin, Rapsyn, nicotinic | qRT-PCR |
| profile - NMJ | AChRs (α1, α7, β1, δ, γ, ε) | |
| Transcriptome | MΥH1, MΥH3, MΥH7, MΥH8, TNNT1, TNNT3, | |
| profile - Muscle | TTN, TPM1, and RYR1 | |
| NMJ function | Response to neuronal stimulants and AChR | Contraction analysis in the |
| | blockers. | assay as described herein |

For analysis of spatially separated cultures, muscle-only and neuron-only controls are generated to better evaluate the effect of the neuronal presence. All analyses are blinded by a third party to ensure the achievement of robust and unbiased results. Significant differences between groups are evaluated using appropriate statistical tests (e.g., ANOVA with post-hoc Tukey's tests for multiple comparisons) and performed on SigmaPlot™ statistics software. A p value of less than 0.05 is considered significant.

Without wishing to be bound by theory, it is proposed that spatially separated culture of healthy muscle with hiPSC-derived motor neurons bearing different ALS mutations can lead to significant differences in synaptic function. Furthermore, structural and gene expression markers of NMJ and muscle development can be more poorly expressed in cultures supporting mutant neurons. Defects in NMJ functionality, as measured using the assay as described herein, are expected to be observed across all ALS mutations. However, there can be differences in the degree of functional defect and level of response to treatment with agonists and antagonists. Without wishing to be bound by theory, it is expected that differences in NMJ functional development across different ALS mutations can correlate with the clinical severity observed in patients with the same genetic defect.

Mutant neurons may still be able to form NMJs with comparable functionality to healthy controls when paired with healthy muscle. If this is observed, a more complex culture system can be used comparing healthy neurons and healthy muscle with mutant neurons and mutant muscle to test what impact these mutations have on the development of NMJs within the system. This requires the use of hiPSC-derived skeletal muscle and previously published protocols can be used to achieve this (see e.g., Chal J et al. Nat Protoc. 2016, 11:1833-50; Chal et al. Nat Biotech. 2015, 33:962-9; each of which is incorporated by reference herein in its entirety). In such a model, a comparative matrix can be performed wherein healthy and diseased neurons are each separately paired with both healthy and diseased muscle. Cultures are analyzed for the metrics, e.g., as described in Table 3, with NMJ function assessed using the assay as described herein. Skeletal muscle satellite cells derived from ALS patients have been shown to exhibit inhibited differentiation potential in culture suggesting that ALS-relevant mutations may impact muscle development directly. As such, it is expected that diseased neurons and diseased muscle spatially separated cultures exhibit a more pronounced functional and molecular phenotype then diseased neurons on healthy muscle.

### Example 3

Assessing the Capacity for Novel Therapeutic Compounds to Restore Normal Synaptic Function in Nerve-Muscle Spatially Separated Cultures

Although few approved compounds exist for the treatment of ALS, a number of drugs are currently being tested to assess their efficacy and toxicity in treating this debilitating condition. Assessment of these compounds within the proposed preclinical assay and comparison of resulting data with clinical outcomes therefore represents an exciting opportunity to validate the engineered platform in terms of its capacity to mirror results achieved in patients.

Mexiletine is a use-dependent sodium channel blocker that has been used for decades for the treatment of cardiac arrhythmias and recently its use has been expanded to the treatment of neuropathic pain in diabetic polyneuropathy. Mexiletine has been shown to be protective of neurons following spinal cord, head injury, and cerebral ischemia, largely by blocking excitotoxicity. A phase 2 clinical trial is investigating whether Mexiletine can ameliorate symptoms in ALS patients (see e.g., Clinical Trials Identifier: NCT02781454, accessible on the world wide web at clinicaltrials.gov/ct2/show/NCT02781454). As such, results from the in vitro assay as described herein can be compared with incoming clinical data directly and therefore confirm the capacity for this assay to faithfully recapitulate human drug responses.

The potassium channel opener Retigabine is being investigated in a phase 2 clinical trial to assess the compound's effects on neurophysiological function in upper and lower motor neurons from ALS patients (see e.g., Clinical Trials Identifier: NCT02781454, accessible on the world wide web at clinicaltrials.gov/ct2/show/NCT02450552; see e.g., Wainger et al. Cell Rep. 2014, 7:1-11. Data with another potassium channel opener (Nicorandil), has shown that such compounds can act to limit mitochondrial production of reactive oxygen species (ROS) in human cells (see e.g., Afzal et al. J Cardiovasc Pharmacol Ther. 2016, 21: 549-62), indicating a possible therapeutic mechanism for these compounds. Preliminary analysis of ROS production in TDP-43 Q331K mutant motor neurons suggests such ALS-relevant mutations may increase ROS production as part of their pathology (see e.g., FIG. 9A-9C) and provide further support for the testing of these compounds in the in vitro platform described herein. Counter to these data, it has long been known that the slow potassium channel blocker 3,4-diaminopyridine (DAP) enhances acetylcholine release from the nerve terminal and improves conduction in unmyelinated nerves (see e.g., Aisen et al. Journal of the neurological sciences. 1995, 129:21-4). As such, it has been suggested that this compound may help functionality in ALS patients. Given these conflicting results arguing both for potassium channel openers and blockers in the treatment of ALS, the high throughput platform as described herein is used to test which compounds are best able to improve NMJ interactions.

The device as describe as herein is used to investigate the ability for mutant neuron-muscle spatially separated cultures to revert to a normal phenotype following treatment with small molecules currently undergoing clinical trials (e.g., Mexilitine and Retigabine) as well as some with similar modes of action (e.g., Nicorandil) or conflicting modes of action (e.g., DAP). These data not only validate the platform as a means to evaluate novel ALS therapeutics but also offers additional support for the use of compounds currently in clinical trials in the treatment of this debilitating condition.

Culture of ALS Patient hiPSCs

In addition to CRISPR edited lines, e.g., generated as in Example 2, the functionality of hiPSC populations derived directly from ALS patients is investigated so that results can more closely be linked to results from clinical trials. Human iPSC lines can be purchased from biobanks, e.g., the Target ALS™ (accessible on the world wide web at targetals.org/) and Answer ALS™ (accessible on the world wide web at answerals.org/) biobanks. Patient cells can be obtained from such biobanks with known mutations in TARDBP, SOD1 and C9orf72. These hiPSC populations are then be expanded and subjected to the motor neuron differentiation and purification protocols, e.g., as defined in Example 1. Immunostaining, FACS, and patch clamp can be employed to verify these cells' phenotype prior to use within the experimental assay.

### NMJ Functionality and Response to Drug Therapy

Human iPSC motor neurons derived from mutant lines edited with CRISPR methods as well as patient lines can be established within the compartmentalized system along with muscle cells derived from isogenic control hiPSCs. NMJ functionality can first be assessed across a 3-week time course, e.g., as detailed in Example 1. Once phenotypes are established for each patient line, the application of drug therapy following treatment can be investigated. Compounds are applied from culture inception, and synaptic function is compared with that of cultures given drug treatment after 7 and 14 days in vitro, as well as untreated culture controls. In this way, it is possible to assess whether extended drug exposure confers an additive benefit to neuromuscular performance in mutant spatially separated cultures. Synaptic function can be monitored for 3-weeks, with recordings taken every 2-3 days, to determine any improvement compared with untreated controls.

The clinical trials investigating Retigabine and Mexiletine focus on analysis of motor-evoked potential (MEP) threshold, amplitude, and latency. MEPs in clinical neuromuscular evaluation are electrical potentials measured in the muscle by external electrodes in response to neuronal activation (see e.g., Ridding et al. The Journal of Physiology, 2001, 537:623-31). Analysis of the stimulus intensity necessary to evoke MEPs of a certain minimal amplitude provide a threshold measurement and MEP amplitude and latency in response to this stimulus can then be calculated. Since input stimulus can be directly controlled in the NMJ model and contraction amplitude and latency can be measured in the muscle compartment, clinical patient outcomes can be directly compared to results achieved with these compounds within the hiPSC-based assay as described herein.

Experimental groups consist of each CRISPR edited and ALS patient line paired with muscle cells derived from isogenic control hiPSCs. For each line, results are compared with spatially separated cultures established with neurons derived from isogenic controls. For each drug (Mexiletine, Retigabine, Nicorandil, and DAP), experiments consist of untreated, carrier control treated, and then 5 to 7 doses of each compound to be tested. Each dose is tested in separate wells of the high throughput system as described herein. All analyses are blinded by a third party to ensure the achievement of robust and unbiased results. Significant differences between groups are evaluated using appropriate statistical tests (e.g., ANOVA with post-hoc Tukey's tests for multiple comparisons) and performed on SigmaPlot statistics software. In all experiments, a p value of less than 0.05 is considered significant.

Treatment of ALS patient-derived neurons with the described drugs could lead to improvement in functional cross-talk between neurons and muscle cells. The degree of recovery depends on the severity of the phenotype observed in each line, with more mild phenotypes showing a closer return to a normal phenotype. Conversely, cells with the severest in vitro phenotype show the smallest improvements in function following treatment. Treated spatially separated cultures exhibit responses in a dose dependent manner, with higher compound concentrations having a greater functional impact.

Functional variance between investigated lines may prevent direct comparison of drug effects across different genetic backgrounds. Although drug results for each mutant line are compared to results from normal isogenic spatially separated cultures, comparison between lines represents a valuable additional endpoint to help predict the potential utility of the investigated compounds to ameliorate ALS across a wide range of genetic backgrounds. In some embodiments, when variability in baseline function and synapse development across different lines makes direct comparison difficult, experiments focus on a single hiPSC line and use CRISPR-mediated gene editing to generate the specific mutations expressed in the patient lines obtained from the ALS biobanks. In this manner, functional consistency is ensured in controls, facilitating more direct comparisons of the different patient mutations and their response to drug treatment.

As all experiments are conducted using hiPSC-derived neurons and primary muscle cells from a single source, biological variables (sex, age, weight, etc.) should not contribute to variance in the collected data. To ensure confidence in the collected data, described experiments for Examples 1-3 are performed in quadruplicate and repeated at least three times using independently differentiated neurons and skeletal muscle cells. All analyses are blinded and performed by individuals not directly involved in data collection. All statistical analyses are checked and verified through consultation with an independent statistics expert not directly associated with the project.

### Example 4

Bioengineered Human Neuromuscular Junctions for High-Throughput Functional Screening in Peripheral Neuropathic Diseases

Peripheral neuropathies are a clinically and genetically heterogeneous set of severely debilitating neurological conditions with common symptoms being gait disturbance, distal muscle weakness, scoliosis, vocal cord palsy, and respiratory muscle weakness. Among these conditions, Charcot-Marie-Tooth disease (CMT) is the most common inherited peripheral neuropathy, affecting roughly 1 in 2500 people, and is characterized by deformities in the limb extremities, pronounced muscle wasting, scoliosis, and sensory loss. CMT type 1 (CMT1) manifests as a predominantly demyelinating neuropathy, while CMT type 2 (CMT2) typically reflects defects that are intrinsic to axons. All forms of CMT are incurable, and no drugs are available for their treatment. Therapeutic regimens instead focus on a combination of physical therapy, the use of supporting braces and orthopedic surgery. Consequentially, there is an urgent need to develop therapeutic drugs capable of ameliorating CMT symptoms or slowing disease progression in order to improve patient care and quality of life. Greater mechanistic understanding of CMT etiology can permit the identification of suitable biomarkers and therapeutic targets, which in turn leads to the development of treatment modalities. However, genetic variability inherent to CMT pathophysiology makes accurate modeling of the disease difficult, and reduces the predictive power of current preclinical animal and cell-based models.

Described herein are human induced pluripotent stem cell (hiPSC)-based neuromuscular disease models that facilitate detailed analysis of disease progression and comparison of phenotypic differences across varied genetic backgrounds to isolate common features from mutation specific effects. Such models also permit evaluation of patient specific responses to different therapeutic strategies, thereby facilitating the development of more personalized treatment modalities for people suffering from neuropathic conditions such as CMT.

It has been suggested that the neuromuscular junction (NMJ) is an early target for pathological onset in a number of peripheral nerve diseases, and patients with CMT2 commonly possess defects in the maturation of their NMJs. These defects typically precede the onset of progressive muscular degeneration and impairments in pre-synaptic and post-synaptic development in lower motor neuron connectivity to muscle is characteristic of the disease.

A number of genes mutated in NMJ maturation contributes to the subsequent onset of complete denervation and associated muscle weakness. Based on this data, it is clear that the NMJ is an important site of early, selective pathology in axonal CMT. In order to prevent the degradation of synaptic structures, and improve patient mobility, it is vital to understand the upstream effects that lead to CMT2 encoded proteins that influence microtubule-mediated axonal transport, and specifically, mitochondrial transport. These include neurofilament light chain (NEFL), mitofusin 2 (MFN2) and heat shock protein 27 (Hsp27, also known as heat shock protein beta 1 (HSPB1)). In normal vertebrate tissue, mitochondria are enriched at the presynaptic terminal of NMJs. Moreover, synaptic vesicle cluster density along naïe neurites is positively correlated with mitochondrial membrane potential, suggesting that mitochondrial activity may exert a regulatory role in synaptogenesis. Defects in axonal transport within CMT neurons can therefore lead to reduced mitochondrial density at the developing presynaptic terminal, resulting in the formation of severe NMJ malformations. While this mechanism offers one possible explanation for the root cause of CMT2, its consistency across the range of patient mutations known to elicit a CMT2 phenotype has not been established. hiPSC technology makes the analysis of this mechanistic consistency possible in human cells. However, to evaluate the effect of axonal defects on NMJ development using such cell-based systems, a reliable humanized model of NMJ formation and development is required.

In vitro models of NMJ physiology and function are currently available. They typically focus on dual patch analysis or assessment of muscle twitch responses to neuronal activation. However, such systems rely on the formation of immature and transient nerve-muscle contacts and fail to achieve the formation of robust and functionally mature synapses. Such structures are necessary for gaining meaningful mechanistic insight into synapse function and for use in effective disease modeling and drug screening applications. Conventional monolayer cultures poorly replicate the in vivo environment. Therefore, an anisotropic, multilayered tissue culture model (see e.g., Jiao et al. ACS Nano. 2014, 8(5):4430-9, which is incorporated by reference herein in its entirety) can be used with biologically relevant physical and electrical cues to promote improved levels of maturation, leading to an in vitro system suited for promoting NMJ development and the study of its breakdown in disease.

Described herein are NMJ assays, including a structurally organized 3D model of innervated human skeletal muscle and its use to analyze the underlying mechanism leading to the breakdown of synaptic structures in a wide range of peripheral neuropathies, such as for example CMT2 patients. Human iPSC-derived motor neurons from multiple axonal CMT patients are integrated with the structured muscle model to investigate whether mutations with varying severities of axonal defects exhibit notable differences in terms of NMJ development. Such a system can demonstrate the feasibility of generating patient specific disease models for subsequent therapy evaluation and mechanistic studies. Chemical modulators of axon stability (histone deacetylase 6 (HDAC6) inhibitors) are investigated for their capacity to ameliorate the in vitro phenotype of CMT hiPSC-derived motor neurons within the NMJ model. Such data improves mechanistic insight into CMT2 etiology and demonstrates the utility of the platform as a preclinical screen with which to evaluate the therapeutic potential of new drugs.

The subject matter disclosed herein comprises an in vitro model of axonal CMT neuropathy using hiPSCs. In addition, it incorporates many innovative techniques that contribute significantly to the fields of tissue engineering and stem cell biology in both basic research and clinical settings. The biomimetic platform for developing physiologically advanced, ordered tissues can be of immediate practical relevance to biomedical scientists at large, since these tools can be readily disseminated to the broader biomedical community in order to aid in the advancement of their studies. The presently offered human model system can not only shed new light on the mechanisms underpinning motor neuron dysfunction in axonal CMT, but also provide a suitable test bed for future therapy development studies for NMJ-related diseases including Lambert-Eaton syndrome and myasthenia gravis.

In a further embodiment, besides neuromuscular engineering, the in vitro study of any ordered tissue can benefit from application of the nanopatterned cell sheet engineering technique, since it permits the synthesis of 3D tissues with controllable layered orientations. In this manner, investigators are able to assess the behavior and maturation of more complex tissue structures than is possible with conventional methods. The absence of an exogenous matrix in this system facilitates the generation of more physiologically relevant cell densities, which benefits generation of more biologically relevant tissues for in vitro or in vivo applications.

The devices as described herein can facilitate generation of quantitative descriptions of the relationships between matrix mechanics and cellular development and the effect of electromechanical conditioning on tissue and synapse development in vitro. In some embodiments, hiPSCs are used to generate motor neurons. This permits engineering of tissue from an individual patient, and can be used for the generation of patient-specific transplant material, drug screens, and congenital disease models. In one embodiment, optogenetic neurons can be used to investigate NMJ function. This technique represents an exciting new use of genetically engineered light-activated neurons, which can be of significant benefit for studies of neuron and synapse electrophysiology. The cross-disciplinary nature can foster transformative progress to the understanding of synaptic maturation and development and can ultimately pave the way to developing truly biomimetic human muscle tissue models for both in vitro and in vivo applications.

The devices and systems described herein involve ordered muscle tissue engineering, micro/nano-technology, biomaterials, tissue engineering, stem cell biology, and neuromuscular physiology. In some embodiments, the devices can be used to investigate whether hiPSC-derived motor neurons carrying different CMT2 patient mutations exhibit distinct structural, electrophysiological, and axon transport properties in vitro (see e.g., Section 1 below). Without wishing to be bound by theory it is proposed that, despite different underlying mutations, hiPSC-derived neurons from CMT patients exhibit a degree of consistency in terms of structural, functional, and axonal transport defects. The functionality of these neurons can be investigated in terms of their capacity to generate synaptic contacts with primary human myotubes within a 3D skeletal muscle model (see e.g., Section 2 below). Although primitive synaptic contacts have been demonstrated in in vitro systems previously, the provision of biomimetic electromechanical conditioning cues as described herein enhances synaptic development and permits stratification of CMT disease phenotypes. The severity of NMJ malformation can correlate with the degree of structural and axonal transport defects observed in neurons with different mutations. CMT patient hiPSC-derived motor neurons can be analyzed in isolation, and within the 3D NMJ model, to gauge the effect of HDAC6 inhibitors on ameliorating CMT disease phenotype in vitro (see e.g., Section 3 below). HDAC6 deacetylates microtubules and thereby diminishes stability of axonal microtubules. Consequently, HDAC6 inhibitors improve axon stability and have been shown to enhance mitochondrial transport in cultured rodent neurons. The devices as described herein can be used to analyze whether these improvements carry over to human cells, and whether improvements in axonal transport can restore synaptic development within a humanized model of CMT2. The data can demonstrate the utility of the engineered constructs as a suitable system in which to screen therapeutic compounds for their capacity to ameliorate the CMT phenotype, and provide data to develop HDAC6 inhibitors towards a clinically relevant therapy for CMT2 patients.

1. Evaluation of the In Vitro Phenotype of Motor Neurons Generated from Patient-Derived Stem Cells

The phenotypic analysis of motor neurons derived from axonal CMT patient hiPSCs highlights a level of consistency in terms of the structural, molecular, and functional defects that occur in these cells, but that phenotype severity varies based on the underlying mutation present. An ability to reproduce the physiological and functional hallmarks of CMT2 accurately permits stratification of phenotypes between cells derived from different patients and normal controls, leading to the establishment of more effective drug screening and disease-modeling platforms.

Four hiPSC lines have been generated from patients carrying CMT2A (MFN2 gene mutation), CMT2E (NEFL gene mutation) and CMT2F (HSP27 gene mutations, S135F and P182L) genotypes. Investigations of the electrophysiological profiles of these cells demonstrates that motor neurons with different patient mutations possess significantly slower motor nerve conduction velocities (MNCVs) compared to wild-type (WT) counterparts (see e.g., Chung et al. Brain: a journal of neurology. 2006, 129(Pt 8):2103-18). MNCVs for WT human motor neurons are greater than 50 m/s, but motor neurons derived from patients carrying MFN2 gene mutations display an average MNCV of 41.0±23.7 m/s. Similarly, those with NEFL gene mutations have mean MNCVs of 44.0±8.8 m/s, while neurons with HSP27 gene mutations (S135F and P182L) show average MNCVs of 48.3±17.8 m/s. Interestingly, S135F and P182L mutations within the same HSP27 gene, show different mitochondrial transport characteristics (data not shown). Patients carrying an S135F mutation in the HSP27 gene exhibit motor and sensory nerve dysfunction, but patients with the P182L mutation develop only motor nerve dysfunction. Functional studies of the motor neurons from each patient's hiPSC-derived neurons revealed that the absolute velocity of mitochondrial movements was significantly decreased in S135F-motor neurons compared with WT controls. However, no significant difference between P182L-motor neurons and controls was detected (S135F-motor neurons; 0.19±0.01 μm/sec and P182L-motor neurons; 0.22±0.01 μm/sec vs. WT-motor neurons; 0.24±0.01 μm/sec) (data not shown). Furthermore, the proportion of moving mitochondria was significantly decreased in P182L-motor neurons compared to controls, but not in S135F-motor neurons (S135F-motor neurons; 26.37±5.01%, P182L-motorneurons; 14.64±2.14%, and WA09-motor neurons; 31.39%±3.74) (data not shown). These data serve to highlight that iPSC-derived motor neurons from axonal CMT patients display functional phenotypes, and that specific mutations lead to significant differences in the functional properties of the differentiated cells. These data highlight that axonal transport defects observed in rodent models of CMT2 and known to be present in patients carrying these mutations can be observed in cultured human cells. The devices as described herein can be used to acquire phenotype characterization and comprehensive data on the properties of CMT2 neurons.

hiPSC-derived motor neurons can be produced that exhibit typical neuronal morphology and electrophysiological function, and stain positive for motor neuron specific markers such as HB9 and Tuj1 (data not shown). Further, comprehensive structural, molecular, and functional characterization of hiPSC-derived CMT motor neurons can yield a greater understanding of the similarities and differences in CMT2 phenotypes, and permit identification of common defective pathways that can offer therapeutic targets.

Consequentially, hiPSC-derived motor neurons with varied CMT2 genotypes can be subjected to a wide array of physiological characterization using devices as described herein. The collected data can demonstrate that neurons carrying varied CMT mutations exhibit common defects in structural, electrophysiological, and axon transport properties, but that severity of these phenotypes varies across examined mutations, thereby permitting CMT2 subtype stratification. Identification of the metrics with the strongest correlation to genotype can provide the basis for subsequent characterization when new patient hiPSCs are derived.

Generation of Axonal CMT Patient hiPSCs

Axonal CMT patient iPSC lines have been generated carrying four different mutations in three CMT2 related genes (MFN2, NEFL, and 2 separate HSP27 gene mutations, S135F and P182L). Experiments demonstrate the reliability of the differentiation method for use with both WT and diseased donor cells (data not shown). Briefly, skin fibroblasts were obtained from 4 mm-punch skin biopsies collected from control patients and two patients carrying S135S or P182L mutant forms of HSPB1(Hsp27) following IRB approval and informed consent. Fibroblasts cultures were established from collagenase/trypsin/Dispase digested fresh skin samples using conventional methods. Four Sendai viral vectors expressing Sox2, Oct4, Klf4, and c-Myc were introduced into fibroblasts from control as well as patient cells. After overnight transduction, cultures were maintained for 6 days and then cells were trypsinized and replated onto mitomycin C-treated mouse embryonic fibroblasts (MEF), SNL feeder cells, and harvested with ESC/iPSC medium containing 4 ng/mL basic fibroblast growth factor (bFGF). The medium was changed daily. At day 30, iPSC colonies were selected based on their characteristic morphology. Sox2, Oct4, Klf4, and c-Myc expression were analyzed in all cell populations by qRT-PCR in order to verify successful reversion to a stem cell phenotype.

Differentiation of hiPSCs into Motor Neurons

Human iPSCs from the four (4) patient lines described above, as well as familial WT controls, are maintained on gelatinized tissue culture plastic on a monolayer of irradiated CF-1 mouse embryonic fibroblasts. An hiPSC medium, consisting of DMEM: nutrient mixture F-12 with 20% knockout serum replacer, 110 μM β-mercaptoethanol L-glutamine, nonessential amino acids and 20 ng/mL basic fibroblast growth factor (bFGF), are used to maintain the cells; this medium is changed every 24 hours. These cells are differentiated to form motor neurons over a course of 30 days, essentially as described by Amoroso et al. (see e.g., Amoroso et al. The Journal of neuroscience, 2013, 33(2):574-86).

Characterization of Cultured hiPSC-Derived Motor Neurons

Following differentiation and subsequent culture, motor neuron phenotype is verified by staining for motor neuron specific markers HB9 and Islet-1 (early markers of motor neuron identity), as well as choline acetyltransferase (ChAT), Tuj1, and SMI32 (late markers) (see e.g., FIG. 11). Percent purity of differentiated motor neuron populations is calculated from confocal images of the stained cells. In addition to purity counts, the number of cells expressing early vs late motor neuron markers is compared as an indication of population maturity. Cultures are immunostained for markers of axons (neurofilament) and dendrites (MAP2). Axon length and dendritic length and branching are quantified from immunostained images and any defects in axonal or dendritic structure are recorded. Quantitative western blot data are used to evaluate expression of mutant proteins in comparison to WT controls, to assess whether alteration of target protein expression levels is observed.

Functionality of the differentiated motor neurons is evaluated using whole-cell patch clamp electrophysiology. Electrophysiological metrics to be analyzed and compared include rate of spontaneous action potential firing, inward Na⁺ and outward K⁺ current amplitudes, resting membrane potential, action potential peak amplitude, full width half max assessment of action potential duration, and time to peak amplitude. In addition, activation response to glutamate, GABA, and glycine can be characterized and compared to previously reported data for stem cell derived motor neurons (see e.g., Miles et al. The Journal of neuroscience. 2004, 24(36):7848-58).

Mitochondrial transport along axons is evaluated using live cell imaging of cultured neurons. Cells are infected with a lentiviral vector expressing mitochondrially targeted fluorescent probe dsRed (mito-dsRed) permitting real-time assessment of mitochondrial movement. Cells are placed within an environmental chamber mounted on an epifluorescent microscope and mitochondrial movement is recorded for 10 minutes with 3-second time-lapse intervals. Image analysis is performed with ImageJ, and Matlab code can be used, for example, to calculate mitochondrial velocity by measuring the distance between the position of individual mitochondria at the start and end of time-lapse recordings and dividing by the time elapsed. This provides a measure of overall transport velocity that includes anterograde and retrograde movements and stationary periods. Mitochondria are then classified as motile (velocity >0.1 μm s-1) or stationary (velocity ≤0.1 μm s-1), in line with disclosed methods (see e.g., Saporta et al. Experimental neurology. 2015; 263:190-9). Immunostaining is used to quantify mitochondrial density within the developing growth cone of cultured neurons as a means to evaluate the effect of altered transport on presynaptic development. To account for time dependent maturation, neurons are evaluated for all end-points following 1, 2, 3, and 4 weeks in culture. Results are compared across genotypes and time points, in order to evaluate differences in maturation rate over time and across genetic backgrounds.

All methods and analyses are performed in triplicate, and repeated at least three times using independently differentiated neurons. Groups can comprise each CMT patient line and familial WT controls. All analyses are blinded by a third party to ensure the achievement of robust and unbiased results. Significant differences between groups are evaluated using appropriate statistical tests (ANOVA with post-hoc Tukey's tests for multiple comparisons) performed on SigmaPlot statistics software, for example. A p value of less than 0.05 is considered significant.

Axonal CMT neurons can display significantly poorer axonal transport properties than do WT controls. Specifically, results can show an increase in the number of stationary mitochondria, and reduced speeds in motile mitochondria. The severity of this reduction in transport can vary across genetic backgrounds, with more severe deficits occurring in cells from patients with more pronounced CMT2 phenotypes. Similarly, electrophysiological parameters can show some differences between CMT neurons and WT controls. Changes in action potential thresholds, time to peak depolarization, and repetitive firing behavior, as indicated by data from isolated CMT hiPSC-derived neurons (see e.g., Saporta 2015 supra) can be observed.

In some embodiments, lentivirally-expressed mitoDsRed can be used to effectively labels mitochondria in iPSC-derived motor neurons. The mito-dsRed may, however, directly alter axonal transport properties in cultured neurons. In some embodiments, the dye MitoTracker Red CMXRos can be used to stain mitochondria in live cells. Comparison of transgenic mitochondrial reported lines with WT controls, in terms of electrophysiological and structural endpoints, can be carried out to ensure that the introduction of the reporter molecule had no significant effect on overall phenotype, and ensure that any differences observed are due to the underlying CMT mutation.

### 2. Evaluation of NMJ Formation in Multilayered Motor Neuron-Myotube Models of Multiple CMT Subtypes

In addition to functional defects in the neuron, axonal CMT NMJs in vivo display persistent defects in maturation in terms of number of post-synaptic malformations, AChR plaque size and complexity, and AChRϵ-subunit expression. However, conventional monolayer cultures of motor neurons and myotubes do not typically support robust formation of NMJs, and therefore are ill suited to stratification of disease phenotypes. Furthermore, there is evidence that synaptic breakdown is influenced by nerve-muscle electrical activity, indicating that activation rates of the NMJ may influence synaptic maintenance in peripheral neuropathic disease states. Without wishing to be bound by theory, it is proposed that proper motor innervation requires replication of skeletal muscle structure, as a three-dimensional aggregate of appropriately oriented myotubes, and the provision of physiologically relevant electromechanical cues.

Myotubes cultured on nanopatterned surfaces promote increased levels of cellular maturation, in terms of the number of nuclei per myotube, and upregulation of myogenic regulatory factors (MRFs) responsible for promoting myoblast differentiation and fusion into myotubes (data not shown). These data, coupled with the increased alignment achievable with such anisotropically patterned substrates, indicate that nanopatterned substrates promote the formation of more developmentally mature and physiologically representative skeletal muscle tissues. Experiments with C2C12 myotubes on thermoresponsive nanofabricated substrates (TNFS) highlight that aligned skeletal muscle monolayers established on nanopatterned substrates retain their anisotropic morphology when transferred onto flat surfaces (see e.g., Jiao 2014 supra). Similarly, human myotubes derived from primary tissue are capable of aligning on TNFS and transferring successfully to new substrates. Multilayered C2C12 constructs maintain layer specific orientations when stacked in a helical structure, indicating that initial anisotropic guidance cues are sufficient to exert a lasting influence over cell sheet orientation, even after removal of the substrate signal and its replacement with confounding signals from oblique cell layers (data not shown). The ability for ordered skeletal muscle constructs to retain their structural anisotropy upon detachment demonstrates the feasibility of producing multilayered human skeletal muscle constructs with persisting anisotropic structural characteristics.

Maintenance of skeletal muscle myotubes within organized 3D formats is a means of enhancing skeletal muscle maturation in vitro. However, incorporation of hydrogels or other scaffolds, as is typically relied upon for 3D culture, prevents the development of physiologically relevant cell densities and the generation of biomimetic cell-cell contacts. The TNFS method as described herein is scaffold-free, and as such permits the synthesis of more accurate representations of skeletal muscle. As such, this platform represents an ideal device with which to evaluate the in vitro phenotype of CMT2 neurons in terms of their ability to form NMJs.

Data with hiPSC motor neurons reveals formation of preliminary synaptic contacts when these cells are co-cultured with C2C12 myotubes (data not shown). Although the structure of these “en passant” synapses suggests immaturity, these data represent a suitable starting point from which to investigate the development and maturation of in vitro NMJs through modulation of the microenvironmental niche. In some embodiments, described herein are human/rodent hybrid co-cultures. In some embodiments, described herein are human/human co-cultures. Modulation of electrical pacing and mechanical strain can promote the physiological and functional maturation of NMJs within the 3D engineered muscle tissues. Electromechanical conditioning improves skeletal muscle maturation, and these combined physiological stimuli in vitro can promote maturation of the cultured cells toward a more adult phenotype, thereby permitting the development of more mature and structurally robust synaptic structures. Furthermore, maintenance of NMJs in vivo is activity dependent, as such correct recapitulation of electrical stimulation can enhance synaptic development in vitro.

Primary human myotubes and WT (WA09) hiPSC-derived motor neurons can be used in TNFS-based 3D models of innervated skeletal muscle. The methods for generating stacked muscle constructs can be adapted to support motor neuron survival. Given the activity-dependent nature of NMJ maturation in vivo, the effect of electromechanical stimulation on NMJ development can be analyzed within the platform to evaluate whether provision of such biologically relevant stimuli enhances synaptic development in vitro. In some embodiments, the devices described herein are used to evaluate how different CMT2 neuron phenotypes, in terms of axonal structure and transport properties can lead to significant differences in the degree of NMJ malformation within the engineered constructs.

Differentiation of Primary Myoblasts into Skeletal Muscle Myotubes and Phenotypic Characterization

Primary human myoblasts from Lonza™ are used to generate multilayered muscle constructs. These cells are plated at low density in Lonza™ growth medium. Once confluent, these cells are switched into a differentiation medium, consisting of 3% horse serum and 10 ng/mL insulin-like growth factor 1 in high glucose DMEM, to induce differentiation into myotubes. Myotube cultures are maintained in this medium for a further 7 to 10 days prior to analysis. Undifferentiated myoblasts are characterized by immunocytochemistry for the muscle specific structural marker desmin. Confocal images of these stains are used to calculate population purity. Experiments with these cells highlight at least 80% purity across multiple passages, indicating the stability of this cell population across multiple experiments. Gene expression patterns is characterized by qRT-PCR. Expression of Pax7, Myf5, MyoD, and myogenin can be assessed to ensure the cultured myoblasts possess correct transcription profiles. Myoblasts are then pushed into terminal differentiation to form myotubes as described above. These myotubes are examined for the development of striated sarcomeres and contractile function to provide further evidence of the suitability of the described methods for producing functional skeletal muscle from these primary human precursors.

### TNFS Fabrication and Characterization

Fabrication of nanotopographical substrates utilizes epoxy-amine chemistry, which allows for the spontaneous reaction between epoxy groups and primary amine groups (data not shown). Specifically, a polyurethane acrylate (PUA) and epoxy-containing glycidyl methacrylate (GMA) solution is mixed together and utilized in conjunction with capillary force lithography to fabricate nanotopographical substrates. A thermoresponsive polymer, poly(Nisopropylacrylamide) (PNIPAM) is grafted to the surface of the nanofabricated substrate to facilitate sheet detachment. PNIPAM changes from a hydrophobic hydrogel above its lower critical solution temperature (LCST) of 32° C., capable of supporting cell adhesion and growth, to a hydrophilic polymer below the LCST, which permits controlled cell sheet detachment. Thus, cells can be cultured as monolayers at typical culture temperatures and then spontaneously detached as a cell sheet by lowering the culture temperature. To allow for PNIPAM grafting, the nanotopographical substrate is incubated with an amine-terminated PNIPAM solution in DI H₂O and allowed to react for 24 hours. To confirm nanotopography fidelity, SEM can be used to image the TNFS, and X-ray photoelectron spectroscopy can be used to analyze their elemental composition.

Transfer of Nanopatterned Cell Sheets to Create 3D Structures

Cell sheets contract upon detachment from the substrate surface. Consequentially, a suitable method to transfer these sheets without loss of cell viability, morphology, or alignment is required. For this purpose, a gel casting method can be utilized (see e.g., Jiao 2014 supra). Briefly, the TNFS is incubated with room temperature PBS, and then melted gelatin is added to the substrate and allowed to cool at 4° C. The casted myoblast cell sheets and gelatin are then removed from the TNFS and transferred to a new surface. Transferred sheets are allowed to attach at 28° C., before being transferred to 37° C., which melts the gelatin, releasing the cell sheet. Using this method, multiple layers of cell sheets can be stacked on top of each other. The formation of 3-layer constructs has been validated to generate 3D ordered cultures.

In some embodiments, TNFS variables can be optimized. A range of PNIPAM grafting densities have been investigated (e.g., 1, 5, 10, 15, 20, 25, 50 and 75% GMA in PUA), as well as PNIPAM molecular weights of 4,500 and 7,500 grams/Mole. Cell sheet detachment and maintenance was compared when TNFS was pretreated with 0.2% gelatin, 5 μg/mL fibronectin, or 100% fetal bovine serum (FBS). In some embodiments, a 1% GMA solution, 4,500 g/M PNIPAM, and pretreatment with FBS can provide very reliable results, in terms of successful sheet detachment. In some embodiments, the device can comprise different co-culture ratios of myoblasts to motor neurons, e.g., to determine optimal conditions for NMJ formation. Ratios of 1:10, 1:100, and 1:1000 (motor neuron: myoblast) are examined for their capacity to form detachable sheets with high rates of cell survival. For each of these conditions, successful muscle sheet formation and detachment are confirmed via bright field microscopy. Actin immunostaining is analyzed to determine maintenance of structural anisotropy following detachment and live/dead assays, in conjunction with MAP2 staining, is used to confirm and quantify motor neuron survival.

### Fabrication and Characterization of Multilayered, 3D Skeletal Muscle Tissues

To fabricate innervated 3D muscle tissues, primary myoblasts are allowed to form confluent monolayers on TNFS substrates as described above. Human iPSC-derived motor neurons are then plated on top of these cultures in different ratios (described above) and maintained for a further two days to ensure attachment. During co-culture, cells are maintained in the neuronal maintenance medium as this contains the necessary conditions to ensure muscle survival (e.g., high glucose medium, with provision of sufficient serum derived factors). Following co-culture establishment, the described gel casting method is used to cast the first sheet. The medium from the second sheet is then removed, and the gelatin plus first sheet (sheet side down) is stacked on top of the second sheet (still attached to the TNFS) at room temperature and allowed to adhere for 30 min. An orientation key will be used to layer the subsequent sheets in parallel with the first (data not shown). In some embodiments, systems comprising 1, 3, 5, and 7-layer muscle-motor neuron tissues are analyzed to determine thickness limits via a fluorescent viability assay 24 hours after transfer.

This approach produces motor neurons embedded within skeletal muscle tissue, which is a scenario that does not exist in nature. This approach may promote formation of NMJs. Therefore, in one approach a monolayer of motor neurons is overlaid with 3, 5, or 7 layers of myotubes. This approach has the advantage that culture media composition and differentiation times can be controlled separately for motor neurons and myotubes.

In vivo, each motor axon innervates multiple myotubes (the so-called motor unit) by branching as it grows orthogonally to the orientation of the myotubes. As motor axons grow oriented parallel to the striations of the nanopatterned substrate, the myotubes layers can be oriented orthogonally to the axon tracts. For each culture scenario, tissue structure can be assessed 7 and 21 days after transfer (to account for time dependent maturation) through immunostaining for actin fibers and markers of muscle and motor neuron development.

Assessment of NMJ development

NMJ formation in WT constructs can be assessed by immunocytochemistry. Average acetylcholine receptor (AChR) plaque diameter, degree of post-synaptic membrane invagination, number of co-localized pre- and post-synaptic structures, and number of poly-innervated vs. singularly innervated vs. denervated fibers are quantified via stains for AChRs (post-synaptic marker) and synaptic vesicle proteins (pre-synaptic markers). NMJ functionality can be assessed through application of a neuronal stimulant (glutamate) and quantification of changes in muscle contractile activity (see e.g., Smith et al. Technology. 2013; 1(1):37-48). For this analysis, particle image velocimetry (PIV) and digital image correlation (DIC) algorithms (see e.g., Milde et al. Integrative biology. 2012; 4(11):1437-47) can be used to quantify contractile responses from bright field video recordings. In some embodiments, software (see e.g., Macadangdang et al. Cellular and molecular bioengineering. 2015; 8(3):320-32) divides a reference video frame into a grid of windows of a set size. Each window is run through a correlation scheme with a second frame, providing the new location for that window in the second frame. This displacement is converted into a vector map, which provides contraction angles and, when spatially averaged, contraction magnitudes and velocities. The correlation equation used provides a Gaussian correlation peak with a probabilistic nature that provides sub-pixel accuracy. For analyses, a video capture system with a frame rate above 24 frames per second (fps) can be used. Following glutamate treatment, addition of a neuromuscular blocker (D-tubocurarine) can then be used to investigate whether the established synaptic contacts respond correctly to NMJ antagonists. Differences in sensitivity to increasing doses of both glutamate and D-tubocurarine, in terms of their capacity to stimulate/block muscle contraction, can be evaluated as a measure of increased maturation of the synapses present in culture.

### Establishment of Optogenetic Motor Neuron Cultures for NMJ Activity Assessment

The glutamate and D-tubocurarine methods for evaluating NMJ function offer a method to evaluate synaptic activity. However, this system does not allow for controlled neuronal activation, which is vital for detailed assessment of NMJ function. For these reasons, systems described herein can comprise optogenetic hiPSC-derived motor neurons (see e.g., Jazayeri et al. Nature neuroscience. 2012; 15(10):1368-70), so that light pulses can be employed to depolarize the neuron and assess subsequent myotube contraction. Briefly, an adeno-associated virus (AAV) vector delivers the channelrhodopsin-2 gene (ChR2; rAAV1-hSyn-ChR2(H134R)-mCherry) into cultured neurons. Absorption of a photon by neuronally expressed ChR2 proteins generates a large permeability for monovalent and divalent cations, thereby depolarizing the membrane and triggering an action potential. Viral particles are generated via the well-established helper-free triple-transfection procedure (see e.g., Cao et al. Gene therapy. 2002; 9(18):1199-206), dialyzed in PBS, and titered on cultured hiPSC motor neurons to identify a suitable concentration that ensures a high degree of infectivity. The capacity for transfected cells to respond to light activation can be verified by whole cell patch clamp. Gap free recording in current clamp mode can be used and the cells exposed to different durations and intensities of blue light pulses. This assay not only verifies expression of the ChR2 protein, but also it identifies suitable stimulation parameters for subsequent experiments. Integration of the hSyn (human synapsin-1) promotor restricts ChR2 expression to neurons, and inclusion of the mCherry protein permits identification of successfully transfected cells in culture. Motor neuron-myotube co-cultures are transfected for 3 hours and then given fresh medium for 1 day, before being assessed for NMJ function. Blue light pulses are delivered to neuronal constructs at regular intervals, and video recordings is used to assess myotube contractions in response as described previously (see e.g., Steinbeck et al. Cell Stem Cell. 2016; 18(1):134-43). Pulse widths and intensities optimized during preliminary patch experiments are used to elicit contractions. The number of pulses required to generate muscle twitches is compared between conditions as a measure of NMJ functional maturity. In robust NMJs, a single neuronal action potential promotes a depolarizing end-plate potential in the myotube. However, in more transient contacts, further neuronal depolarizations may be required. As such, the number of blue light pulses required to produce muscle twitches will be quantified as a measure of NMJ maturity. 2 ms light pulses are initially delivered at 1 Hz. If muscle contractions are not also observed at the same rate, 2, 5, 10, 20, and 50 Hz light pulses are tested, and the number of contractions observed in response will be recorded.

### Electromechanical Conditioning of Innervated Muscle Tissues

In order to assess the effect of electromechanical stimulation on the establishment and maturation of in vitro NMJs, the 3D co-culture system can be integrated with a bioreactor platform to permit synchronous broad-field electrical stimulation and mechanical stretch stimuli. Innervated muscle tissues are engineered as described above as multilayered aligned constructs. Transferred tissues are placed within a FlexCell™ Bioflex™ 6-well plate and allowed to attach. The gelatin is melted at 37° C. and washed away to leave multilayered skeletal muscle constructs attached to the Bioflex™ membrane. The FlexCell™ system has a flexible membrane substrate that deforms and stretches under vacuum and can be used to stretch the transferred tissues (preload). Constructs are subjected to cyclic and ramp strain protocols over fixed periods in line with parameters for skeletal muscle constructs. Briefly, cells are subjected to 15% stretch (relative to their initial length). Stimulation occurs at a rate of 1 Hz (0.5 seconds of deformation alternating with 0.5 seconds of relaxation).

To allow for simultaneous electrical stimulation of multilayered tissues, a C-Pace EP system is used. The C-Pace electrodes are fitted to 6-well plate covers and are thus compatible with the FlexCell™ system. The combined FlexCell™ and C-Pace bioreactor can be configured such that the field stimulation occurs during maximal strain of the substrate. Stimuli are applied as 40 ms square wave pulses at 10, 15, and 20 Hz. Each 40 ms pulse is followed by a 10 ms pulse pause before the next stimulus is applied. This regimen is maintained for 2 hours, followed by 8 hours of rest. This protocol can be repeated until experimental endpoints are reached. Conditioned tissues are cultured for periods of 7 and 21 days before analysis in order to determine whether constructs undergo time-dependent maturation. Immunocytochemical and contraction analyses (using PIV and DIC algorithms) are used as described above to evaluate synapse structure and function respectively.

### Assessment of NMJ Development in Axonal CMT Co-Culture Constructs

Three-dimensional axonal CMT motor neuron-muscle co-cultures are established using protocols optimized for WT controls. In addition to structural and functional endpoints discussed above, qRT-PCR can be performed to evaluate γ- to ϵ-AChR subunit switch, which is an established measure of NMJ maturation and known to be inhibited in peripheral neuropathies. Cells derived from all four axonal CMT patient lines are compared in order to evaluate degrees of variability in disease phenotype between these iPSC sources. This variability can be compared to that seen between patients carrying the examined mutations, to evaluate whether phenotypic variability, is recapitulated in the in vitro system.

In some embodiments, systems and methods are performed in triplicate, and repeated at least three times using independently differentiated neurons and separate batches of primary muscle cells. For characterization of the 3D constructs, groups comprise single layer untransferred co-cultures and single layer transferred co-cultures. These are compared to multilayered constructs of varying thickness in order to determine whether the 3D environment has a positive impact on structure and function. For each condition described, muscle-only controls are generated in order to evaluate the effect of neuronal presence. A final control condition is unpatterned 2D co-cultures to evaluate whether structural development provided by underlying topography enhances synapse development. Groups for electromechanical conditioning experiments comprise unconditioned, stretched only, electrically paced only, and stretched and electrically paced constructs. Again, muscle-only controls for each condition are evaluated to provide information on the importance of neuronal presence. For CMT, groups are defined by the use of neurons derived from different CMT patient hiPSCs. Data are compared to familial WT controls. All analyses are blinded by a third party to ensure the achievement of robust and unbiased results. Significant differences between groups are evaluated using appropriate statistical tests (ANOVA with post-hoc Tukey's tests for multiple comparisons) and performed on SigmaPlot statistics software, for example. A p value of less than 0.05 is considered significant.

Co-culture of primary myoblasts with hiPSC-derived motor neurons can generate detachable cell sheets that retain the anisotropic orientations generated through contact with underlying nanotopographies. As tissue thickness is limited by oxygen diffusion of roughly 80 μm-100 μm, and stacked cell sheets have an average sheet thickness of 15 μm-20 μm, the tissue thickness limit is expected to be 5 layers. Multilayered constructs more closely recapitulate the tissue structure of native skeletal muscle and thereby provide a more biomimetic environment for synapse formation and maturation. Therefore, 3D constructs with optimal cell ratios and engineered parameters can promote high levels of pre- and post- synaptic marker co-localization, indicating preliminary synaptic contact. In addition, co-culture of WT neurons with 3D aligned skeletal muscle constructs can promote greater structural development of in vitro NMJs, and improvements in their sensitivity to neuronal stimulants, NMJ blockers, and blue light pulses (in optogenetic experiments). Application of electromechanical conditioning to these cultures can further enhance synaptic development in an activity-dependent manner. Combinatorial electromechanical conditioning can have a greater effect than either stimulus applied in isolation. CMT2 neurons, in co-culture with electromechanically conditioned muscle, can exhibit poorer NMJ formation and functionality than WT controls. Specifically, CMT2 neurons are expected to exhibit poorer sensitivity to stimulants and blockers, a reduction in post-synaptic membrane complexity, and reduced expression of AChRϵ-subunit mRNA. The degree to which CMT2 neurons differ from WT controls can correlate with the severity of the in vitro phenotype observed in Section 1, highlighting that axonal defects in these cells directly affects their ability to form synapses.

Transferred tissues may detach from the glass and lose structure, making prolonged maintenance of these multilayered structures problematic. In this case, an alternative culture strategy is adopted which allows for the casting of muscle sheets or multilayered tissues within a thermally stable hydrogel such as fibrin. For imaging, the casted sheets are processed using conventional histological techniques to produce sections that can then be imaged using confocal or bright field microscopy. Given the predicted impaired development of axonal CMT2 motor neurons, it is possible these cells are not sufficiently robust to withstand detachment and stacking into multilayer constructs. If this is the case, the described experimental condition can be exclusively employed in which purely myotube monolayers are stacked atop an undisturbed motor neuron monolayer. It is also possible (albeit unlikely, given the axonopathic nature of CMT2) that no phenotype is observed when using WT human myoblasts as the muscle source with CMT2 motor neurons. If this is the case, hiPSC-derived myoblasts can be produced to permit co-culture of CMT2 motor neurons with CMT2 myotubes. This design permits comparisons of diseased muscle with WT motor neurons in order to investigate the possibility of retrograde signaling from muscle to neuron in the development of CMT2 pathophysiology. Lastly, variation in ChR2 vector transfection levels could potentially lead to considerable variability in the responsiveness of cultured hiPSC-derived motor neurons to blue light pulses. Such an issue would make accurate assessment of muscle contraction in response to increasing rates of stimulation problematic. In this case, a more stable transfection method can be established for the production of ChR2 expressing hiPSCs. Stable transfection of the ChR2 trans-gene can obviate issues with variable expression.

### 3. Assessment of the Capacity for HDAC6 Inhibitors to Alleviate Axonal CMT Pathological Traits in Vitro

HDAC6 is a tubulin-deacetylating enzyme that modulates axonal growth and development of the axon initial segment; a morphological development that establishes a diffusion barrier to distinguish axon and somatodendritic compartments. Improved acetylation of microtubules in cell and animal based models of Parkinson's disease through treatment with HDAC6 inhibitors has been shown to restore axonal transport deficits and locomotive behavior. Furthermore, HDAC6 inhibition ameliorates loss of synaptic function in a mouse model of CMT2, highlighting its potential suitability as a therapeutic target for axonopathic CMT. Work in hiPSC-derived neurons has shown that HDAC6 modulates post-translational modification of β-catenin, leading to changes in adherens junctions at the cell membrane, providing further insight into how these inhibitors stabilize axonal development. Despite the promise of HDAC6 inhibitors to ameliorate axonal degradation in conditions like CMT2, its effect on human NMJ development has yet to be evaluated.

The screening system as described herein offers a unique model with which to gauge the effect of such compounds on NMJ development in CMT2 patient hiPSC-based cultures. Data collected provide further support of the upstream mechanisms leading to NMJ breakdown in CMT2, and also validate the platform as a preclinical screening platform for evaluating the therapeutic potential of new drugs.

Treatment with the HDAC6 inhibitor Tubastatin A significantly improved levels of acetylated α-tubulin in CMT2 neurons (data not shown). Treatment with this compound was also found to improve both the absolute velocity and the total number of moving axonal mitochondria. Decreased acetylated α-tubulin abundance is a characteristic feature of CMT2, and has been directly linked to severe axonal transport deficits in mouse models of the disease. The in vitro data show concordance with in vivo data, and therefore supports that treatment of engineered human CMT2 tissues with HDAC6 inhibitors is a viable option for ameliorating the CMT2 phenotype in vitro. The ability to alter CMT2 phenotypes in cultured iPSC motor neurons suggests that HDAC6 treatment can have a similar positive effect on other metrics of neuronal and NMJ development. The NMJ platform constitutes the ideal model for performing assessment of whether the reported changes result in restoration of WT NMJ phenotypes. Furthermore, the establishment of methods for screening compound action using this platform is translatable to other drug candidates and models of other neuropathic disease states.

The 3D NMJ co-culture platform as described herein can be used to investigate the ability for HDAC6 inhibitors to alleviate CMT2 phenotypes. The collected data demonstrate the importance of restoring axonal transport properties as a means to preventing NMJ breakdown in CMT2, as well as validate the use of the platform as a preclinical screening system for therapeutic compounds. Alteration in the disease phenotypes of 3D motor neuron-myotube constructs when exposed to HDAC6 inhibitors can be compared to data from WT controls and untreated diseased constructs in order to ascertain compound efficacy. Data is collected for treated and untreated samples across WT and all four CMT2 patient derived iPSC neuron populations described herein, in order to assess the level of variability in compound responses from cells carrying different patient mutations. Significant variance in response to HDAC6 inhibitor treatment in neurons carrying different CMT2 mutations can affirm the need for developing personalized medical screening platforms with which to optimize treatment concentrations and strategies for complex genetic conditions like CMT2.

CMT hiPSC-Neuron Based Co-Culture Set Up

Cells are differentiated and cultured as described above. Three-dimensional constructs are generated and subjected to maturation protocols described in Section 2 above. The HDAC6 inhibitor Tubastatin A (5 μM) is incorporated into the medium from the establishment of the co-culture, and maintained until endpoint analysis. The constructs are investigated after 7 and 21 days to evaluate additive effect of treatment over time. The constructs can also be assessed at these time points after 24-hour treatment to evaluate responses that are more acute. Lastly, 21-day-old co-cultures given Tubastatin A can be investigated for the last 7 days of culture to evaluate whether the compound is capable of reversing the phenotype of cells established over the previous 14 days of culture.

Evaluation of HDAC6 Effect in CMT Patient hiPSC-Derived Neurons

All four previously defined axonal CMT hiPSC lines (MFN2, NEFL, and HSP27 mutants) are investigated. Assessment of NMJ structure and function is analyzed using immunocytochemistry, PIV and DIC algorithm-based contractile analysis, optogenetic functional screens, and qRT-PCR as previously described. In addition, axonal transport in isolated neurons are assessed with and without Tubastatin A treatment to verify directly the effect of treatment on neuronal phenotype. Likewise, immunocytochemical analysis of axon and dendrite structure is performed, and patch clamp electrophysiology is employed to ascertain whether improvements in mitochondrial transport and axon growth correlate with improved electrophysiological function.

In some embodiments, tests are performed in triplicate, and repeated at least three times using independently differentiated neurons and separate batches of primary muscle cells. Groups comprise each CMT2 patient line and familial WT controls engineered into optimized and matured 3D platforms as dictated by the parameters in Section 1. Neurons are also investigated in isolation using standard tissue culture conditions. All analyses are blinded by a third party to ensure the achievement of robust and unbiased results. Significant differences between groups are evaluated using appropriate statistical tests (ANOVA with post-hoc Tukey's tests for multiple comparisons) and performed on SigmaPlot statistics software. In all experiments, a p value of less than 0.05 is considered significant.

Treatment of neurons and co-cultures with Tubastatin A can ameliorate the defects in neuronal physiology and NMJ structure and development observed in untreated cells. The degree to which WT phenotypes are restored can vary depending on the mutation present in the cultured cells. Cells with the severest in vitro phenotype can show the smallest improvements in phenotype. The concomitant analysis of Tubastatin A responses in both isolated neurons and the tissue-engineered NMJ model provides a degree of assurance that meaningful data is achieved

The proposed concentration of Tubastatin A was decided based on preliminary studies. However, this may be adjusted. If treatment at this concentration provides poor results, a dose response study can be performed using axonal transport changes in isolated neurons as the primary metric. This permits optimization of treatment parameters prior to scaling up the experiments to include the engineered 3D tissues.

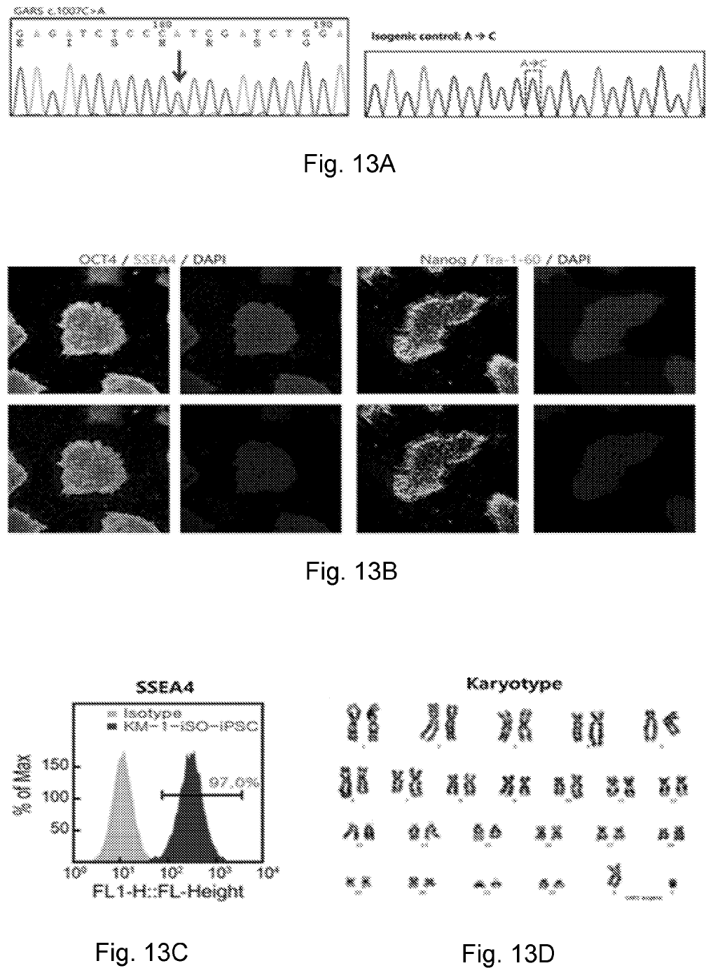

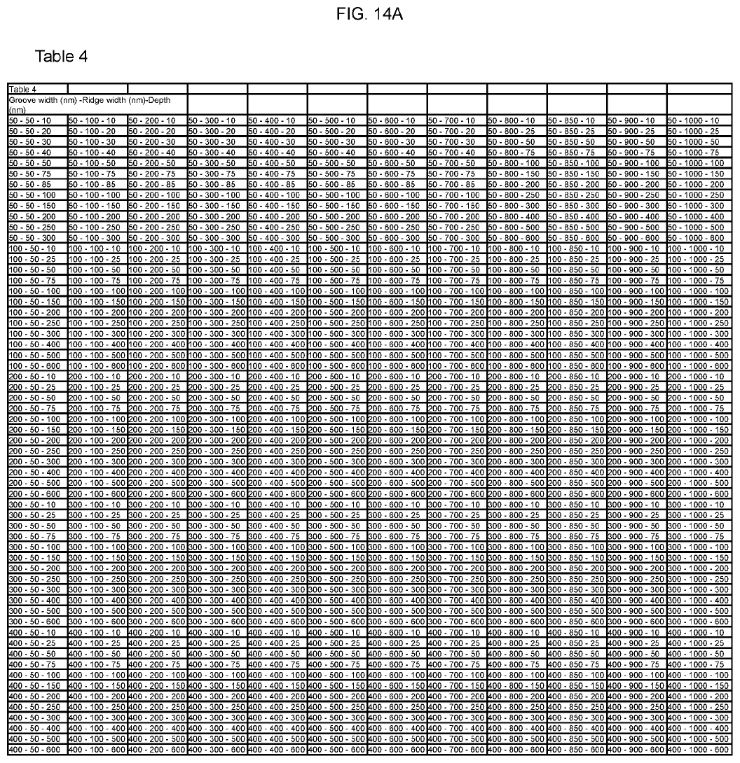

Induced pluripotent stem cell (iPSC) populations derived from patients carrying a point mutation in the GARS gene and exhibiting a Charcot Marie Tooth (CMT) type 2D clinical phenotype were generated and characterized. These cells can be differentiated into human motor neurons for functional assessment. Immunocytochemical analysis indicated that the differentiation method successfully produced Islet-1 positive motor neurons capable of extending neuritic processes out into their surrounding culture environment (see e.g., FIG. 12A). In order to assess the functional capacity of these cells, the percentage of cells exhibiting different action potential firing capabilities were quantified by whole cell patch clamp electrophysiology (see e.g., FIG. 13B). Cells derived from GARS mutant iPSCs exhibited poor firing capabilities in response to 500 ms depolarizing stimuli, with very few cells able to produce multiple action potentials. In contrast, healthy control populations contained cells with far greater capacity to fire repetitive action potentials (see e.g., FIG. 12C).

To further improve the cultured neuronal populations, proliferating cells can be removed from culture following differentiation. In early cultures, these cells quickly outcompeted non-proliferative neuronal cells and led to cultures with few surviving neurons after 30 days. The neuromuscular co-cultures require significant time in vitro to develop mature synapses, and so the means to ensure long-term survival of the neuronal populations is essential. Described herein is a method to facilitate purification of neurons through negative antibody selection of cells expressing CD44 and CD184. Magnet activated cell sorting (MACS) using antibodies against these surface markers created highly enriched neuronal populations with roughly 70% Islet-1 positivity and almost 100% expression of the pan neuronal marker MAP-2 (see e.g., FIGS. 12D, 12E). These MACS purified cells were stable in culture beyond 50 days in vitro and during this time developed dense axonal and dendritic networks indicated by neurofilament and MAP-2 staining (see e.g., FIGS. 12F, 12G). Electrophysiological assessment of can be performed on the GARS neurons at 50 days in vitro.

Differences in functional performance between control and GARS mutant cells can be attributable to differences in genetic background. A familial control iPSC line for the GARS mutants was provided. However, the neurons differentiated from these iPSCs were not stable in culture and could not be maintained long-term. This may be attributable to the age of the donor tissue when the biopsy was taken as the sample was provided by the patient's grandmother when she was in her 70 s. To account for differences in age and genetic background, CRISPR-Cas9 gene editing was used to correct the point mutation present in the CMT2D patient derived line to create an isogenic control for further experimentation (see e.g., FIG. 13A). The generated isogenic control cells stained positive for standard markers of pluripotency, and FACS analysis confirmed the high degree of stem cell purity in these populations (see e.g., FIGS. 13B, 13C). Karyotyping confirmed that the edited line did not suffer any significant genetic damage as a result of the editing process (see e.g., FIG. 13D). Differentiation and functional characterization of the isogenic control line can be performed in parallel with electrophysiological assessment of the GARS mutant cells at 50 days in vitro.

C2C12 cells have been used to conduct extensive characterization of the aligned cell sheet transfer method in terms of its ability to facilitate differentiation of myoblasts to produce 3D, cell-dense skeletal muscle tissues. In doing so, several interesting observations were made regarding the ability for transferred cells to produce their own ECM network that is retained following cell sheet transfer. The persistence of this organized ECM network facilitates maintenance of the organized cellular arrangement originally promoted by the nanotopographic substrates. Triggering differentiation of the layered myoblasts into myotubes permits fusion of cells from different sheet layers, creating different myotube morphologies depending on sheet alignment. ECM transferred with the cells may maintain layer specific alignments until fusion is triggered. The release of matrix metalloproteinases known to accompany skeletal muscle differentiation likely leads to a breakdown of these ECM layers facilitating fusion of myoblasts between layers. The collected data highlight the ability to generate engineered skeletal muscle with physiologically-representative alignments and cell densities. These engineered tissues can be used in engineering the neuromuscular co-cultures with GARS mutant and isogenic human iPSC-derived motor neurons later in the project.

1. A device for monitoring the electrical communication between two different electrically excitable cell types, comprising at least one module on a substrate, each module comprising:
a. a first cell growth area, a second cell growth area, and an axon outgrowth area flanked between the first cell growth area and the second cell growth area, and
b. a plurality of field potential electrodes and a plurality of impedance electrodes on the surface of the substrate,
wherein the plurality of field potential electrodes are located on the surface of the first cell growth area and the plurality of impedance electrodes are located on the surface of second cell growth area, and
wherein the plurality of field potential electrodes and the plurality of impedance electrodes are connected to an electronic interface.

2. The device of claim 1, wherein the axon outgrowth area has a width of at least 100 μm between the first cell growth area and the second cell growth area.

3. (canceled)

4. (canceled)

5. The device of claim 1, wherein the axon outgrowth area comprises a series of parallel microchannels with a proximal and distal end, wherein the proximal end of the microchannels interfaces with the first cell growth area, and the distal end of the microchannels interfaces with the second cell growth area.

6. (canceled)

7. The device of claim 1, further comprising at least one barrier located between the first cell growth area and the second cell growth area, wherein the barrier is configured to separate a plurality of cell bodies of cells located on the first cell growth area from cells located on the second growth area.

8. The device of claim 7, wherein the barrier is configured as any one or more of:
a. located at the interface between the first cell growth area and the axon outgrowth area,.
b. located within the axon outgrowth area, and wherein the barrier is configured to separate a plurality of cell bodies of cells located on the first cell growth area from cells located on the second growth area,
c. barrier is configured to allow axons from cells located on the surface of the first cell growth area to extend into the axon outgrowth area,
d. a non-removable or removable physical barrier, or
e. same width as the axon outgrowth area.

9.-12. (canceled)

13. The device of claim 1, wherein the device comprises at least one of:
the plurality of field potential electrodes (FPE) is arranged in an array,
the plurality of impedance electrodes (IE) is arranged in an array,
the field potential electrodes (FPE) are configured to receive an electrical signal via the electrical interface from a power source and configured to deliver an electrical stimulating signal to the surface of the first cell growth area
wherein the field potential electrodes (FPE) are configured to monitor any one of: spontaneous, electrically-paced, or optically-paced activity of cells in contact with the field potential electrode.

14.-16. (canceled)

17. The device of claim 13, wherein the field potential electrodes (FPE) are configured for one or more of:
a. monitoring any one of: spontaneous, electrically-paced, or optically-paced activity of cells in contact with the field potential electrode; and
b. electrically stimulating cells present on the first cell growth area,
c. electrically stimulating the cells on the second growth area, wherein the electrical stimulation is mediated by stimulating the cells on the first growth area and the synaptic connections of the cells on the first growth area permitting the transmission of the signal to the cells present on the second growth area.

18. (canceled)

19. The device of claim 1, wherein the impedance electrodes (IE) are communicatively coupled via the electrical interface to at least one analyzing module in the form of an impedance analyzer, thereby permitting impedance monitoring from excitable cells attached to the surface of the second growth area.

20. The device of claim 1, further comprising a third cell surface area, wherein an edge of the third cell surface area interfaces with a proximal edge of the first cell growth area and the axon outgrowth area, or a distal edge of the second cell growth area and the axon outgrowth area.

21. The device of claim 1, further comprising a plurality of neuronal cells on the first cell growth area and a plurality of contractile cells or muscle cells on the second cell growth area.

22. (canceled)

23. The device of claim 21, wherein the plurality of neuronal cells or muscle cells are derived from iPSCs obtained from a healthy subject or a subject with a neurodegenerative disease or disorder or a myopathy.

24. (canceled)

25. The device of claim 21, wherein the plurality of neuronal cells and plurality of skeletal muscle cells are selected from any of:
a. motor neurons, and skeletal muscle cells
b. sympathetic neurons and smooth muscle cells, or
c. sympathetic neurons and cardiac muscle cells or cardiomyocytes.

26. (canceled)

27. (canceled)

28. The device of claim 21, further comprising an additional cell type on any one or more of: the first cell growth area, the second cell growth area or the axon outgrowth area, wherein the additional cell type is selected from any of: Schwann cells, microglia, astrocytes or satellite cells.

29. (canceled)

30. (canceled)

31. (canceled)

32. The device of claim 21, wherein the neuronal cells on the first cell growth area extend axons through the axon outgrowth area and into the second cell growth area comprising a plurality of muscle cells.

33. (canceled)

34. The device of claim 1, wherein the first cell growth area comprises a nanopatterned surface, or the second cell growth comprises a nanopatterned surface, or both the first and the second cell growth surfaces comprise a nanopatterned surface, wherein the nanopatterned surface provides anisotropic cues that promote improved levels of maturation of neuronal cell types, motor neurons and myocytes.

35. (canceled)

36. (canceled)

37. (canceled)

38. The device of claim 1, comprising an array of modules on the substrate.

39. (canceled)

40. A method for measuring the electrical conductance from one cell type to a second cell type comprising:
a. providing a device of claim 1, wherein the device comprises a first cell type on the first cell growth area, and a second cell type on the second cell growth area, and wherein the first cell type extends axons across the axon outgrowth area to the second cell type in the second cell growth area;
b. providing electrical stimulation to the first cell type via the field potential electrodes, and
c. recording electrical activity of the second cell type via the impedance electrodes.

41. The method of claim 40, wherein the first cell type is a plurality of neuronal cells or a plurality of neuronal cells derived from iPSCs obtained from a healthy subject or a subject with a neurodegenerative disease or disorder or a myopathy.

42. The method of claim 40, wherein the second cell type is a plurality of contractile or muscle cells, or a plurality of muscle cells derived from iPSCs obtained from a healthy subject or a subject with a neurodegenerative disease or disorder or a myopathy, or a plurality of muscle cells in a monolayer or as an engineered skeletal muscle construct.

43.-48. (canceled)

49. The method of claim 4, wherein the plurality of neuronal cells, or plurality of muscle cells, or both, are selected from:
genetically modified cells to introduce one or more mutations for a neurodegenerative disease or myopathy, or
isogenic controls of a genetically modified cell that has one or more mutations introduced for a neurodegenerative disease or myopathy.

50. (canceled)

51. The method of claim 40, further comprising assessing the electrical conductance across at least one neuromuscular junction (NMJ) between an axon extended from the first cell type and the cell bodies of the second cell type.

52. An assay for assessing an agent for modulation of electrical signaling from one cell type to another cell type, comprising:
a. providing a device of claim 1, wherein the device comprises a first cell type located on surface of the first cell growth area, and a second cell type located on the second cell growth area, and wherein the first cell type extends axons across the axon outgrowth area from the first cell area to the second cell type in the second cell growth area;
b. contacting the first cell type, second cell type, or both, with an agent;
c. providing electrical stimulation to the first cell type via the field potential electrodes;
d. recording electrical activity of the second cell type via the impedance electrodes; and
e. detecting a change in electrical activity of the second cell type recorded via the impedance electrodes in the presence of the agent as compared to the absence of the agent.

53.-63. (canceled)
